SEQUENCE LISTING (21 sequences)

SEQ ID NO: 1 - DNA, 57 bp, Artificial sequence
  Synthetic
ccggcaacaagatgaagagcaccaactcgagttggtgctcttcatcttgttgttttt

SEQ ID NO: 2 - DNA, 57 bp, Artificial sequence
  Synthetic
ccggcctagcccttattgcattgttctcgagaacaatgcaataagggctaggttttt

SEQ ID NO: 3 - DNA, 53 bp, Artificial sequence
  Synthetic
gatggatgaattgcagttgttctcgagaacaactgcaattcatccatcttttt

SEQ ID NO: 4 - DNA, 25 bp, Artificial sequence
  Synthetic
aaaacctagcccttattgcattgtt

SEQ ID NO: 5 - DNA, 21 bp, Artificial sequence
  Synthetic
gatggatgaattgcagttgtt

SEQ ID NO: 6 - DNA, 7052 bp, Artificial sequence
  Synthetic
ttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcacgaaggctgtagacaaatactgggacagctacaaccatcccttcagacaggatcagaagaacttacatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtccgcaggtatgcacgcgtgaattctcgacctcgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggccgcggctcgaggggg

SEQ ID NO: 7 - DNA, 4974 bp, Artificial sequence
  Synthetic
aagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacaggatccggtcgcgcgaatttcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgactgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggcgagcctgaattcgggggggggggggggggggacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccggccaacggtctggtgacccggctgcgagagctcggtgtacctgagacgcgagtaagcccttgagtcaaagacgtagtcgttgcaagtccgcaccaggtactgatatcccaccaaaaagtgcggcggcggctggcggtagaggggccagcgtagggtggccggggctccgggggcgaggtcttccaacataaggcgatgatcatcgatgctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggcgagctcgaattaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 8 - DNA, 160 bp, Artificial sequence
  Synthetic
gggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctcctttt

SEQ ID NO: 9 - DNA, 180 bp, Artificial sequence
  Synthetic
agtcgcgtggcatttgtgcctcgtcagaacatgtttcgcgacaacagcggggaggaagctgaggaaatgcgagactgcaggtttagggccggtcgcgagctgcgccgcggatttaatcgcgagcgactgctgcgtgaggaggactttgagccagatgaacattcggggattagttctgca

SEQ ID NO: 10 - DNA, 163 bp, Artificial sequence
  Synthetic
ggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagcccctttttt

SEQ ID NO: 11 - DNA, 163 bp, Artificial sequence
  Synthetic
gctttaataaccatgtgcgcacactaatagcgcgagaagaagtagccattggtttaatgcatctttgggactttgtagaagcttatgtacataatccagcaagtaaacccctaactgcccagctgttcttaatagttcaacatagtagagacaatgaaacttt

SEQ ID NO: 12 - DNA, 21 bp, Artificial sequence
  Synthetic
cctagcccttattgcattgtt

SEQ ID NO: 13 - DNA, 21 bp, Artificial sequence
  Synthetic
gatggatgaattgcagttgtt

SEQ ID NO: 14 - DNA, 19 bp, Artificial sequence
  Synthetic
gattggggtgcttgtttgg

SEQ ID NO: 15 - DNA, 19 bp, Artificial sequence
  Synthetic
ttcccatttcatcaggtgc

SEQ ID NO: 16 - DNA, 24 bp, Artificial sequence
  Synthetic
catgtttgtgatgggtgtgaacca

SEQ ID NO: 17 - DNA, 24 bp, Artificial sequence
  Synthetic
gttgctgtagccgtattcattgtc

SEQ ID NO: 18 - DNA, 30 bp, Artificial sequence
  Synthetic
aagcggaggaggttcttaaaacctcacaaa

SEQ ID NO: 19 - DNA, 30 bp, Artificial sequence
  Synthetic
ggatccctaccacatacgccttttatcacc

SEQ ID NO: 20 - RNA, 160 nt, Artificial sequence
  Synthetic
gggcacucuuccguggucugguggauaaauucgcaaggguaucauggcggacgaccgggguucgagccccguauccggccguccgccgugauccaugcgguuaccgcccgcgugucgaacccaggugugcgacgucagacaacgggggagugcuccuuuu

SEQ ID NO: 21 - RNA, 163 nt, Artificial sequence
  Synthetic
agcgggcacucuuccguggucugguggauaaauucgcaaggguaucauggcggacgaccgggguucgaaccccggauccggccguccgccgugauccaugcgguuaccgcccgcgugucgaacccaggugugcgacgucagacaacgggggagugcuccuuuu

(19) United States
(12) Patent Application Publication
(10) Pub. No.: US 2016/0002643 A1
(43) Pub. Date: Jan. 7, 2016

(54) Compositions And Methods To Enhance Protein Expression

(71) Applicant: Board of Regents, The University of Texas System, Austin, TX (US)

(72) Inventors: Jaquelin P. Dudley, Austin, TX (US); Hyewon Byun, Austin, TX (US); Mary M. Lozano, Austin, TX (US)

(73) Assignee: The Board of Regents, The University of Texas System

(21) Appl. No.: 14/730,878

(22) Filed: Jun. 4, 2015

Related U.S. Application Data
(60) Provisional application No. 62/104,447, filed on Jan. 16, 2015.
(60) Provisional application No. 62/019,596, filed on Jul. 1, 2014.

(51) Int. Cl. C12N 15/63; A61K 39/12; C12N 7/00; C12N 15/113; A61K 48/00; A61K 39/145; C12N 15/86

(52) U.S. Cl. CPC C12N 15/635; A61K 39/145; A61K 39/12; C12N 15/86; C12N 15/113; C12N 15/1131; A61K 48/0066; C12N 7/00; C12N 2310/531; C12N 2310/14; C12N 2320/30; C12N 2740/15043; C12N 2760/16034; C12N 2770/24134; A61K 2039/53

(57) ABSTRACT

The present invention relates to methods, compositions and kits for increasing expression of transfected genes through the use of super-induction nucleic acid sequences, including highly structured RNA. In particular, co-transfection of super-induction nucleic acid sequences including, for example, shRNA encoding sequences, retroviral elements, and (in other embodiments) VA-RNA encoding sequences, is contemplated for enhancing the expression of co-transfected transgenes in a cell. More specifically, the super-induction sequences are contemplated for use, in one embodiment, with mammalian expression plasmids and gene therapy vectors for increasing transgenic protein activity levels in cells and tissues. In one embodiment, the super-induction sequences of the present inventions are contemplated for use with known DNA vaccination vectors for enhancing therapy or enhancing protective immunity.

This invention was made with government support under Grant no. R01 CA116813 and Grant no. R01 CA167053 awarded by the National Institutes of Health. The government has certain rights in the invention.

### FIELD OF THE INVENTION

The present invention relates to methods, compositions and kits for increasing expression of transfected genes through the use of super-induction nucleic acid sequences, including highly structured RNA. In particular, co-transfection of super-induction nucleic acid sequences including, for example, shRNA encoding sequences, retroviral elements, and (in other embodiments) VA-RNA encoding sequences, is contemplated for enhancing the expression of co-transfected transgenes in a cell. More specifically, the super-induction sequences are contemplated for use, in one embodiment, with mammalian expression plasmids and gene therapy vectors for increasing transgenic protein activity levels in cells and tissues. In one embodiment, the super-induction sequences of the present inventions are contemplated for use with known DNA vaccination vectors for enhancing therapy or enhancing protective immunity.

### BACKGROUND OF THE INVENTION

Virus infection and introduction of foreign nucleic acids induce stress in eukaryotic cells leading to translational arrest. Thus, expression vectors currently used for expressing protein in mammalian cells show low levels of protein expression, particularly in cell types that are difficult to transfect, such as non-dividing cells and terminally differentiated cells, e.g. specialized cells.

Therefore, there remains a need for increasing protein expression from expression vectors used for transfecting eukaryotic cells.

### SUMMARY OF THE INVENTION

The present invention relates to methods, compositions and kits for increasing expression of transfected genes through the use of super-induction nucleic acid sequences, including highly structured RNA. In particular, co-transfection of super-induction nucleic acid sequences including, for example, shRNA encoding sequences, retroviral elements, and (in other embodiments) VA-RNA encoding sequences, is contemplated for enhancing the expression of co-transfected transgenes in a cell. More specifically, the super-induction sequences are contemplated for use, in one embodiment, with mammalian expression plasmids and gene therapy vectors for increasing transgenic protein activity levels in cells and tissues. In one embodiment, the super-induction sequences of the present inventions are contemplated for use with known DNA vaccination vectors for enhancing therapy or enhancing protective immunity.

Advantages include, but are not limited to, constructs and methods that will work in multiple cell types using a variety of transfection methods, as super-induction success appears to be independent of the transfection method. Moreover, it is not necessary to clone sequences corresponding to the highly structured RNA and/or retroviral elements into the same vector from which increased protein expression is desired. While the present invention, in one embodiment, contemplates introducing the highly structured RNA and/or retroviral elements into the vector for which increased protein expression is desired (i.e. using such elements in cis), a preferred embodiment involves no such cloning. Rather, the vector (for which increased protein expression is desired) is simply co-transfected with a vector comprising the highly structured RNA and/or retroviral elements (i.e. using such elements in trans). Multiple sequences can be combined to optimize gene expression in different vectors.

An additional advantage is that one can use a lower amount of the vector for which increased expression of the gene of interest is desired. Use of lower amounts of vector sequences should prevent adverse immunological responses and the development of cancer from retroviral integrations.

In one embodiment, the present invention contemplates a method for transfecting a eukaryotic host cell, said method comprising co-transfecting into said eukaryotic host cell a first vector comprising a gene of interest in operable combination with a promoter, and a second vector comprising DNA sequences corresponding to a highly structured RNA (which, in a preferred embodiment, are not transcribed) so as to create a transfected host cell under conditions such that said gene of interest is expressed in said transfected host cell in an amount that is greater than the level of expression where said first vector is transfected alone. In one embodiment, said highly structured RNA comprises shRNA. Such shRNA need not have a known eukaryotic gene target. In one embodiment, said highly structured RNA comprises VA-RNA. In one embodiment, said VA-RNA is an adenoviral VA-RNA. In one embodiment, said second vector further comprises a retroviral packaging element. In one embodiment, said packaging element is the packaging signal ψ. In one embodiment, said second vector further comprises a Rev-responsive element (RRE). In one embodiment, said second vector further comprises a retroviral 5′ UTR. In one embodiment, said second vector further comprises a promoter upstream of the highly structured RNA. In one embodiment, said promoter is the U6 promoter. In one embodiment, said second vector comprises a multiple cloning site and said U6 promoter is positioned in said multiple cloning site of the second vector.

It is not intended that the present invention be limited by the nature of the second vector. In one embodiment, said second vector is a plasmid vector. In one embodiment, said second vector is a viral vector. In one embodiment, said second vector is a plasmid vector with viral components. In one embodiment, said viral vector is a retrovirus vector. In one embodiment, said retrovirus vector is a lentiviral vector. In one embodiment, said lentiviral vector is not capable of making viral particles. In one embodiment, said retrovirus vector is a gammaretroviral vector. In one embodiment, said gammaretroviral vector is not capable of making viral particles.

It is not intended that the present invention be limited to the nature of the host cell. In one embodiment, said eukaryotic host cell is a primary cell. In one embodiment, said eukaryotic host cell is a non-dividing cell. In one embodiment, the host cell is a stem cell. In one embodiment, said eukaryotic host cell is part of a human tissue and said co-transfecting is done in vivo. In one embodiment, said co-transfecting is done ex vivo and said transfected host cell is introduced into a human in vivo.

It is not intended that the present invention be limited by the nature of the first vector. In one embodiment, said first vector is a plasmid. In one embodiment, the first vector contains a gene of interest, the expression of which is needed to remedy a) a deficiency (e.g. dystrophin gene), including but not limited to immunodeficiencies, or b) a disease such as an infectious disease or a degenerative disease (e.g. neurological degeneration). On the other hand, in one embodiment, said plasmid comprises a DNA vaccine wherein said gene of interest encodes an antigen. In one embodiment, said DNA vaccine is a preventative vaccine for viral infection. In one embodiment, said preventative vaccine is for West Nile virus. In another embodiment, said preventative vaccine is for influenza. In one embodiment, said antigen is the influenza hemagglutinin antigen. In one embodiment, said DNA vaccine is a therapeutic vaccine. In one embodiment, said therapeutic vaccine is a therapeutic cancer vaccine.

As noted above, one advantage is that the super-induction is not limited to any particular mode of transfecting or transfection so as to create a transfected cell. In one embodiment, said co-transfecting is performed by electroporation. In one embodiment, said co-transfecting is performed by needle free jet delivery. In one embodiment, said co-transfecting is performed by lipid-based carriers.

It is not intended that the present invention be limited by the nature of the DNA vaccine used together with the second vector (or modified by cloning to contain the highly structured elements). In one embodiment, said DNA vaccine comprises a eukaryotic region that directs expression of said gene of interest and a bacterial region that provides selection and propagation in bacteria. In one embodiment, said bacterial region provides selection and propagation in E. coli. In one embodiment, said eukaryotic region comprises said promoter upstream, and a polyadenylation signal (polyA) downstream, of said gene of interest. While not limited to any specific promoter, in one embodiment, said promoter is the constitutive human Cytomegalovirus (CMV) promoter.

The second vector can be used with known vector systems for gene therapy. For example, in one embodiment, said first vector is an adenoviral vector or an adeno-associated virus. In one embodiment, said first vector is a vaccinia virus based vector for gene therapy. In one embodiment, said first vector is a plasmid based vector for gene therapy. It is not intended that the present invention be limited to a particular gene of interest. A variety of such genes are contemplated, including but not limited to, genes encoding cytokines, hormones, growth factors, receptors, tumor suppressors, and like.

The present invention contemplates methods where a combination of highly structured RNAs is employed. It is not intended that the present invention be limited to just one combination of elements. Indeed, different combinations of elements for enhanced protein expression in various cell types are contemplated. In one embodiment, the present invention contemplates a method for transfecting a eukaryotic host cell, said method comprising co-transfecting into said eukaryotic host cell a first vector comprising a gene of interest in operable combination with a promoter, and a second vector comprising in operable combination the retroviral packaging signal ψ, the Rev-responsive element (RRE), the U6 (or other) promoter upstream of an shRNA site, and an shRNA site comprising DNA corresponding to shRNA (which, in a preferred embodiment, are not transcribed), so as to create a transfected host cell, wherein said gene of interest is expressed by said transfected host cell in an amount that is greater than the level of expression where said first vector is transfected alone.

The present invention also contemplates compositions. In one embodiment, the present invention contemplates a host cell that has been co-transfected with a first vector comprising a gene of interest in operable combination with a promoter, and a second vector comprising sequences corresponding to a highly structured RNA (said vector can, but need not, express a highly structured RNA). In one embodiment, the structured RNA comprises shRNA (including shRNA with no known gene target). In one embodiment, the structured RNA sequence further comprises multiple retroviral components. In one embodiment, said highly structured RNA comprises VA-RNA. In one embodiment, said VA-RNA is an adenoviral VA-RNA. In one embodiment, the present invention contemplates a host cell that has been co-transfected with a first vector comprising a gene of interest in operable combination with a promoter, and a second vector comprising in operable combination the retroviral packaging signal ψ, the Rev-responsive element (RRE), the U6 (or other) promoter upstream of a shRNA, and a shRNA, so as to create a transfected host cell, wherein said gene of interest is expressed by said transfected host cell in an amount that is greater than the level of expression where said first vector is transfected alone.

The present invention also contemplates kits. In one embodiment, the kit comprises: a) a vector comprising in operable combination the retroviral packaging signal iv, the Rev-responsive element (RRE), the U6 promoter upstream of an shRNA, and a shRNA, and b) instructions for co-transfecting said vector with a second vector comprising a gene of interest (the second vector not included in the kit) under conditions such that said gene of interest is expressed at higher levels than those achieved without co-transfection. In one embodiment, the vector comprising retroviral elements is a plasmid. In one embodiment, the vector (comprising retroviral elements) lacks LTRs. In one embodiment, the vector (comprising retroviral elements) lacks puroR. In one embodiment, the vector lacks the hPGK promoter. In one embodiment, the shRNA has no known human target gene. In one embodiment, the vector further comprises a promoter upstream of said packaging element ψ. In one embodiment, said promoter is the CMV promoter. In one embodiment, said vector further comprises a polyadenylation site downstream of the shRNA. In one embodiment, the polyadenylation site is from the bovine growth hormone (BGH) gene.

In one embodiment, the kit comprises: a) a vector comprising retroviral elements together with highly structured RNA and b) instructions for co-transfecting said vector with another vector comprising a gene of interest (the other vector not included in the kit) under conditions such that said gene of interest is expressed at higher levels than those achieved without co-transfection. In one embodiment, the vector comprising retroviral elements is a plasmid. In one embodiment, the vector (comprising retroviral elements) lacks LTRs, but has the 5′ untranslated region (UTR) of a retrovirus. In one embodiment, the vector (comprising retroviral elements) lacks puroR. In one embodiment, the vector lacks the hPGK promoter. In one embodiment, the vector (comprising retroviral elements) further comprises sequences corresponding to a highly structured RNA (said vector preferably expressing a highly structured RNA). In one embodiment, the highly structured RNA is shRNA, which preferably has no known human target gene. In one embodiment, the retroviral element is packaging element ψ. In one embodiment, the vector further comprises a promoter upstream of said packaging element ψ. In one embodiment, said promoter is the CMV promoter. In one embodiment, said vector further comprises a polyadenylation site downstream of the shRNA. In one embodiment, the polyadenylation site is from the bovine growth hormone (BGH) gene. In one embodiment, the highly structured RNA is a VA-RNA.

In one embodiment, translation is initiated from the same RNA using either a cap or an IRES. In one embodiment, cap-dependent translation is affected by co-transfection of a lentivirus vector. In one embodiment, a mutant of 4E-BP-binding protein was used, which controls cap-dependent translation by eIF4E. This mutant cannot interact or be phosphorylated by mTOR. Co-transfection of this mutant prevents increased expression by the lentivirus vector, confirming that there is a vector effect on cap-dependent translation.

### DEFINITIONS

To facilitate an understanding of the present invention, a number of terms and phrases are defined below:

As used herein, the term “super-induction” refers to a significantly higher protein expression facilitated by super-induction nucleic acid sequences and super-induction vectors incorporating such sequences, as described herein. More specifically, super-induction refers to protein expression at least 2-fold, 3-fold, 4-fold and even 20-fold or greater over protein expression obtained without super-induction nucleic acid sequences. In some embodiments, methods of “super-induction” include co-transfection of an expression vector comprising at least one gene of interest along with a super-induction vector as described herein. In some embodiments, methods of “super-induction” include transfection of an expression vector comprising at least one gene of interest along with super-induction sequences as described herein cloned into the expression vector.

As used herein, the term “vector” refers to a nucleic acid (DNA) construct used to carry a gene of interest into a host organism or from one organism to another, for examples, a vector may be a plasmid based vector; a virus-based vector; a combination of plasmid and virus sequences; a plasmid, a bacmid, a phagemid, a cosmid, a phage, a virus or an artificial chromosome. A vector may be “empty” (at least initially) such that it typically has a multicloning site without an insert of a DNA sequence of interest or gene of interest. In one embodiment, a “vector” also refers to a nucleic acid construct containing a sequence of interest that was subcloned within the vector. The term “vehicle” is sometimes used interchangeably with “vector.” A vector may comprise at least one or more of a promoter sequence.

As used herein, the term “promoter” refers to a region of regulator DNA (i.e. a regulatory element) that initiates transcription of a particular gene, typically located near the 5′ transcription start sites of the gene and in operable combination with at least one gene. The use of an inducible regulatory element (e.g., an inducible promoter) allows for modulation of the production of the gene product in the cell. A promoter includes an inducible promoter, i.e. activated when contacted by a specific substance or compound, such as a BetaGal promoter; a constitutive promoter, i.e. that expresses the sequences of interest constitutively within a cell, such as a human Cytomegalovirus (CMV) promoter; a cell-type specific promoter, a cell cycle specific promoter, a cell maturation specific promoter, and the like. For another example, a promoter specific for myoblast gene expression can be linked to a gene of interest to confer muscle-specific expression of that gene product. Muscle-specific regulatory elements which are known in the art include upstream regions from the dystrophin gene (Klamut et al., (1989) Mol. Cell. Biol. 9:2396), the creatine kinase gene (Buskin and Hausclika, (1989) Mol. Cell. Biol. 9:2627) and the troponin gene (Mar and Ordahl, (1988) Proc. Natl. Acad. Sci. USA. 85:6404).

The term “regulatory element,” as used herein refers to a genetic element, which controls some aspect of the expression of nucleic acid sequences. For example, a promoter is a regulatory element, which facilitates the initiation of transcription of an operably linked coding region. Other regulatory elements are splicing signals, polyadenylation signals, termination signals, etc.

The term “transcription unit,” as used herein refers to the segment of DNA between the sites of initiation and termination of transcription and the regulatory elements necessary for the efficient initiation and termination. For example, a segment of DNA comprising an enhancer/promoter, a coding region, and a termination and polyadenylation sequence comprises a transcription unit.

As used herein, the term “polyadenylation” refers to the addition of a poly(A) tail to the 3′ end (i.e. downstream) of a primary transcript RNA, in other words, a polyadenylation signal (polyA) downstream.

As used herein, the term “circular vector” refers to a closed circular nucleic acid sequence capable of replicating in a host.

As used herein, the term “replicable vector” means a vector that is capable of replicating in a host cell.

As used herein, the term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Because eukaryotic cells are known to utilize promoters, enhancers, and termination and polyadenylation signals, at least one or more of these nucleic acid sequences are included in the expression vector.

As used herein, the term “expression” is intended to refer to translation to a gene product. In the process of expression, a DNA chain coding for the sequence of gene product is first transcribed to a complementary RNA, which is often a messenger RNA, and, in some cases, the transcribed messenger RNA (mRNA) is then translated into the gene protein product.

As used herein, the term “corresponding” as in reference to “corresponding to a highly structured RNA” refers to a DNA (nucleic acid) sequence that after transcription forms a highly structured RNA of the present inventions.

The term “knockdown”, as used herein, refers to a method of selectively preventing the expression of a gene in a host.

As used herein, the tem′. “recombinant DNA molecule” refers to a DNA molecule which is comprised of segments of DNA artificially joined together by means of molecular biological techniques.

As used herein, the term “recombinant protein” or “recombinant polypeptide” refers to a protein molecule which is expressed using a recombinant DNA molecule.

As used herein, the term “plasmid” refers to an artificial construct used as a vector for molecular cloning. A plasmid typically comprises an origin of replication, a selectable marker gene and a cloning site for inserting at least one gene of interest for producing numerous copies of genes and/or proteins of interest when that gene is ligated into the plasmid then transfected into bacteria hosts. In some embodiments, a gene of interest is in operable combination with a promoter sequence.

As used herein, the terms “operably linked,” “in operable combination,” and “in operable order” refer to the attachment of nucleic acid sequences such that they perform their intended function. For example, operably linking a promoter sequence to a nucleotide sequence of interest refers to linking the promoter sequence and the nucleotide sequence of interest in a manner such that the promoter sequence drives the expression of the nucleotide sequence of interest, i.e. causes expression of a polypeptide encoded by the nucleotide sequence of interest. Similarly, operably linking a nucleic acid sequence encoding a protein of interest means linking the nucleic acid sequence to regulatory and other sequences in a manner such that the protein of interest is expressed. The term also refers to the linkage of amino acid sequences in such a manner so that a functional protein is produced.

As used herein, the term “plasmid vector” or “plasmid based vector” refers to an artificial construct whose backbone was derived from a bacterial plasmid. A plasmid vector is used to facilitate the transfer of a gene of interest into a host, through transient or stable transfection.

The terms “reporter gene construct,” or “reporter gene vector,” as used herein refers to a recombinant DNA molecule containing a sequence encoding the product of a reporter gene and appropriate nucleic acid sequences necessary for the expression of the operably linked coding sequence in a particular host organism.

As used herein, the term “reporter plasmid” refers to an artificial construct comprising recombinant genes for providing information on the activity of other genes. A reporter plasmid typically comprises a reporter gene, such as luciferase, GFP, and the like, operably linked to a regulatory element. Regulatory elements for eukaryotic cells include but are not limited to a promoter, an enhancer element, termination and polyadenylation signals.

As used herein, the term “reporter gene” refers to an oligonucleotide (gene) having a sequence encoding a gene product (typically an enzyme or protein), which can be detected and/or quantifiably assayed, and that is not present normally in the research system. A reporter gene typically includes but is not limited to firefly luciferase, Renilla luciferase, any type of green fluorescent protein (GFP), for example, enhanced GFP (EGFP), bacterial genes such as an E. coli lacZ gene encoding beta-galactosidase (lacZ), the bacterial chloramphenicol acetyltransferase (cat) genes, genes encoding beta-glucuronidase (GUS). A reporter gene is typically attached to a regulatory sequence of another gene of interest in order to determine when the other gene would be expressed if it were present.

As used herein, the term “reporter gene expression” refers to monitoring gene expression of a reporter gene, such as in an assay for the expression of the gene of interest.

As used herein, the term “selectable marker” refers to a gene such as a “selectable marker gene” which encodes an enzymatic activity that confers resistance to an antibiotic or drug upon the cell in which the selectable marker is expressed. Selectable markers may be “dominant”; a dominant selectable marker encodes an enzymatic activity, which can be detected in any mammalian cell line. Examples of dominant selectable markers include the bacterial aminoglycoside 3′ phosphotransferase gene (also referred to as the neo gene), which confers resistance to the drug G418 in mammalian cells, the bacterial hygromycin G phosphotransferase (hyg) gene, which confers resistance to the antibiotic hygromycin and the bacterial xanthine-guanine phosphoribosyl transferase gene (also referred to as the gpt gene), which confers the ability to grow in the presence of mycophenolic acid. Other selectable markers are not dominant in that their use is in conjunction with a cell line that lacks the relevant enzyme activity. Examples of non-dominant selectable markers include the thymidine kinase (tk) gene, which is used in conjunction with tk.sup.-cell lines, the CAD gene, which is used in conjunction with CAD-deficient cells and the mammalian hypoxanthine-guanine phosphoribosyl transferase (hprt) gene, which is used in conjunction with hprt.sup.-cell lines. A review of the use of selectable markers in mammalian cell lines is provided in Sambrook et al., supra at pp. 16.9-16.15.

As used herein, the term “virus” refers to an infectious particle or agent that will replicate after it has come in contact with a living cell. A virus may have genetic information as DNA or RNA.

As used herein, the term “viral vector” refers to a virus that was artificially altered by removing and/or adding nucleic acid sequences such that the viral vector is able to move into a host cell or host organism.

As used herein, the term “retrovirus vector” refers to an artificial construct derived from a retrovirus designed to deliver at least one recombinant gene into a host cell. In general, the capacity of a retroviral vector to carry a therapeutic gene or other gene into a cell has a maximum insertion size of around 8 kb. Retroviral vectors target and infect dividing cells and non-dividing cells with a high degree of efficiency of gene transfer and a moderate level of gene expression for long term expression of the transgene. Retroviruses are generally used in an ex vivo gene transfer capacity where the target cells are removed from the patient, stimulated to divide in vitro then transfected with the retrovirus followed by re-administration to the patient. Retroviruses were used in co-infection systems with an adenovirus, a method for allowing retroviral vectors to transduce cells that were normally resistant to that particular retrovirus.

As used herein, the teal′ “retrovirus” refers to a RNA virus that infects cells then replicates through a DNA intermediate, where the reverse transcribed DNA integrates randomly into the host genome. Retroviruses such as Human immunodeficiency virus (HIV) and other lentiviruses infect, i.e. integrate into the genome in non-dividing cells and dividing cells. On the other hand, gammaretroviruses, such as the murine leukemia virus (MLV) infect non-dividing cells at low rates compared to their high rate of infection of dividing cells during mitosis. Other retroviruses have variable rates of infection of non-dividing cells vs. dividing cells.

As used herein, the term “lentivirus vector” or “lentiviral vector” refers to a recombinant nucleic acid sequence comprising lentiviral sequences.

As used herein, the term “lentivirus” refers to a type of RNA retrovirus that can infect both dividing and non-dividing cells, for one example a human immunodeficiency virus (HIV). Major protein components expressed by retroviruses, used for virus replication, are encoded by major transcripts, including gag, pol, env, which code for multiple proteins when post-translationally cleaved by a virus-encoded protease.

As used herein, the term “gammaretroviral vector” refers to a recombinant nucleic acid sequence designed to deliver and express at least one recombinant gene in a host cell.

As used herein, the term “gammaretrovirus” refers to a type of RNA retrovirus that can infect both dividing and nondividing cells, for one example a Murine Leukemia Virus (MLV), another is a Moloney Murine Leukemia Virus (MMMV).

As used herein, the term “herpesvirus” refers to lymphocryptoviruses and rhadinoviruses.

As used herein, the term “transfection” or “transfecting” or “transformation” refers to an introduction of recombinant DNA into cells (e.g. eukaryotic cells and prokaryotic cells) by biophysical or biochemical methods, i.e. methods of transformation. Transformation may be accomplished by a variety of means known to the art including calcium phosphate-DNA co-precipitation, DEAE-dextran-mediated transfection, polybrene-mediated transfection, electroporation, microinjection, liposome fusion, protoplast fusion, lipofection, retroviral infection, needle free jet delivery, lipid-based carriers, and biolistics, see below and in the Examples.

As used herein, the term “co-transfecting” or “co-transfection” refers to introduction of at least two recombinant DNA molecules into cells, for example, at least two vectors, at least two different types of DNA molecules, etc. Another example of co-transfection is “co-infection” or “co-transduction”, whereby at least two infectious viral vectors are introduced into a cell. It is not intended that the term be limited to transfection of two vectors at precisely the same time, although this is convenient.

The term “stable transfection” or “stably transfected” refers to the introduction and integration of recombinant DNA into the genome of the transfected cell. The term “stable transfectant” refers to a cell that has stably integrated recombinant DNA into the genomic DNA.

The term “transient transfection” or “transiently transfected” refers to the introduction of recombinant DNA into a cell where the recombinant DNA fails to integrate into the genome of the transfected cell in the sense that the recombinant DNA will not be passed on to daughter cells. The term “transient transfectant” refers to cells that have taken up recombinant DNA but have failed to integrate this DNA into the genome.

As used herein, the term “host” refers to a cell, tissue, subject, and the like, in other words any living matter which may be transfected with a vector of the present invention.

As used herein, the term “host cell” refers to any eukaryotic or prokaryotic cell (e.g., bacterial cells such as E. coli, yeast cells, mammalian cells, avian cells, amphibian cells, fish cells, and insect cells), whether located in vitro or in vivo which may be transfected with a vector of the present invention.

As used herein, the term “eukaryotic host cell” refers to any host cell comprising a nucleus.

As used herein, the term “human tissue” refers to cells, tissues, organs, etc. derived from or in homo sapiens, such as epithelial, connective, nervous, muscle and bone tissue, and cells obtained from these tissues, in addition to hematopoietic cells in bone marrow, thymus, lymph, blood, and any organ of the body such as brain, liver, pancreas, stomach, etc.

As used herein, the term “in vivo” refers to within a living organism.

As used herein, the term “in vitro” and “ex vivo” refers to outside a living body, such as in an artificial environment.

As used herein, the term “gene therapy” refers to an insertion of a functional gene or another molecule into a host in order to achieve a therapeutic effect.

As used herein, the term “DNA vaccine” comprises a eukaryotic region that directs expression of said gene of interest. Typically, it has a bacterial region that provides selection and propagation in bacteria such as E. coli.

As used herein, the terms “primary cell culture,” and “primary culture,” refer to cell cultures that have been directly obtained from animal tissue. These cultures may be derived from adults as well as fetal tissue.

As used herein, the term “culture,” refers to any sample or specimen, which is suspected of containing one or more microorganisms. “Pure cultures” are cultures in which the organisms present are only of one strain of a particular genus and species. This is in contrast to “mixed cultures,” which are cultures in which more than one genus and/or species of microorganism are present.

As used herein, the term “cell line,” refers to cells that are cultured in vitro, including primary cell lines, finite cell lines, continuous cell lines, and transformed cell lines.

As used herein, the term “cell type,” refers to any cell, regardless of its source or characteristics.

As used herein, the term “non-dividing cell” refers to a cell that is not undergoing actual cell division, i.e. in a quiescent phase, and includes cells in a transient non-dividing state in G₁ (GAP 1) phase or in a more prolonged nonproliferative state, G₀ (Gap ₀) phase, which may be temporary or permanent. When a cell is permanently non-dividing, this G₀ phase is referred to as terminally-differentiated cells such as some neurons and nerve cells, differentiated epithelial cells (i.e., top layers of skin that are still living but do not divide, villus cells of the gut lumen, etc.), muscle cells of the heart; auditory hair cells of the ear; lens cells of the eye, etc. In other word when cells leave mitosis and have completed a division, they enter the G1 phase of the cell cycle and often into a G0 (G zero) phase where at this point they are “growth-arrested”; protein synthesis is decreased as is transcription. Upon stimulation, cells may exit G0 and continue on with the cell cycle, synthesis (S), G2 and leading to division (Mitosis). However, many cells will remain in the G0 state for a long time, such as fibroblasts in the absence of tissue damage, skeletal muscle cells (classic “post-mitotic cells”). For a comparison, human liver cells, in the absence of liver damage, will divide once or twice a year while gut epithelia cells will divide twice a day. The period of quiescence for each type of cell is different, but if it is greater than 48 hours, the method of the subject invention is especially applicable. As examples of quiescent cells are hematopoietic stem cells (CD34+ cells). These cells have the potential to divide and self-renew, but they are normally quiescent until stimulated to divide. These cells are desired targets for gene therapy (sickle cell disease, thalassemia, SCID), and the subject method provides a method to get DNA into the cells even though they normally do not divide.

As used herein, the term “highly structured RNA” refers to RNA capable or having basepairing interactions within a single molecule or set of interacting molecules, such as short hairpin RNA (shRNA). Such RNAs contain self-complementary sequences that allow parts of the RNA to fold and pair with itself to form double helices. Analysis of these RNAs has revealed that they are highly structured. Unlike DNA, their structures do not consist of long double helices but rather collections of short helices packed together into structures akin to proteins.

As used herein, the term “gene of interest” refers to a gene, with or without introns, selected for study or use, such as a gene that encodes an antigen, enzyme, hormone, cytokine, receptor and the like.

The terms “antigen,” “immunogen,” “antigenic,” “immunogenic,” “antigenically active,” and “immunologically active” refer to any substance that is capable of inducing a specific humoral and/or cell-mediated immune response. An immunogen generally contains at least one epitope. Immunogens are exemplified by, but not restricted to molecules which contain a peptide, polysaccharide, nucleic acid sequence, and/or lipid. Complexes of peptides with lipids, polysaccharides, or with nucleic acid sequences are also contemplated, including (without limitation) glycopeptide, lipopeptide, glycolipid, etc. These complexes are particularly useful immunogens where smaller molecules with few epitopes do not stimulate a satisfactory immune response by themselves.

As used herein, the term “vaccine” generally refer to a prophylactic and/or therapeutic substance used to boost the immune system's ability to protect an organism against “foreign invaders,” such as pathogenic and infectious agents, i.e. virally infected cells or bacterially infected cells that may cause disease and for internal protection, such as from abnormal cancer cells. Thus, a vaccine may be a viral vaccine, a bacteria vaccine, or a cancer vaccine and the like.

As used herein, the term “preventive” or “prophylactic” in reference to a cancer vaccine is intended to prevent cancer from developing in a healthy subject or to prevent a specific type of cancer from developing or spreading in a subject.

As used herein, the term “treatment vaccine” or “therapeutic vaccine” in reference to a cancer vaccine refers to administering a treatment to an existing cancer by strengthening the body's natural defenses against the cancer (Lollini, et al., “Vaccines for tumour prevention.” Nature Reviews Cancer 6(3):204-216, 2006).

As used herein, the term “preventive” or “prophylactic” in reference to a viral vaccine is intended to interfere with virus infection or replication in a subject, for example, a preventative vaccine for West Nile virus for preventing a West Nile virus infection or transmission.

As used herein, the term “treatment vaccine” or “therapeutic vaccine” in reference to a vaccine for a pathogen refers to administering a treatment to an existing infection by strengthening the body's natural defenses against the pathogen, such as a pathogenic virus or pathogenic bacteria.

As used herein, the term “influenza” or “flu” refers to an illness caused by RNA viruses of the family Orthomyxoviridae, particularly targeting cells in the nose, throat and lungs.

As used herein, the term “influenza hemagglutinin antigen” refers to an antigenic glycoprotein found on the surface of the influenza viruses.

As used herein, the term “cloning” or “cloned” refers to the process of isolating a nucleotide sequence from a nucleotide library, cell or organism for replication by recombinant techniques. The isolated material is typically inserted into a vector.

As used herein, the term “subcloned” or “subcloning” refers to inserting a linear double stranded gene, such as gene of interest desired for expression, i.e. antigen, hormone, and the like, or linear double stranded nucleic sequence of interest, such as super-antigen sequences, into another double stranded DNA sequence, such as a circular vector, by using at least one endonuclease to open the double stranded DNA sequence, such as within a multicloning site, followed by ligation of the gene of interest into that opening.

As used herein, the terms “polylinker” or “multiple cloning site” refer to a cluster of restriction enzyme sites on a nucleic acid construct, which are utilized for the insertion, and/or excision of nucleic acid sequences.

As used herein, the terms “restriction endonucleases” and “restriction enzymes” refer to enzymes (e.g. bacterial), each of which cut double-stranded DNA at or near a specific nucleotide sequence. Examples include, but are not limited to, Aval, BamHI,

EcoRI, HindIII, HincII, NcoI, Smal, RsaI, etc.

As used herein, the term “restriction” refers to cleavage of DNA by a restriction enzyme at its restriction site.

As used herein, the term “restriction site” refers to a particular DNA sequence recognized by its cognate restriction endonuclease.

As used herein, the term “trans” as in a “trans acting element” or “trans regulatory element” refers to an element which acts upon a gene located on a different DNA molecule, for one example, an element encoded on one vector that acts upon another vector, such as in a co-transfection system of the present inventions where one or more elements on one vector induce expression of genes on another vector in the same cell.

As opposed to “cis” as in a “cis acting element” or “cis regulatory element” or “CRE” refer to a region of noncoding DNA involved with regulating transcription of nearby genes, such as between different parts of the same DNA molecule. A CRE may be a binding site for a transcription factor (including DNA binding proteins), for example, a CRE may be a promoter region that allows binding to an enhancer protein.

As used herein, the term “nucleus” refers to a membrane-enclosed organelle containing chromosomes, found within eukaryotic cells.

As used herein, the term “cytoplasm” refers to a substance, including water, salts, nucleic acids, fats and proteins, which fills a cell and is located between the cell membrane and the nucleus, typically containing structural components such as the organelles, cytoskeleton, and various particles.

As used herein, the term “protein” refers to a nitrogenous organic compound that has one or more chains of 2 or more amino acids linked by peptide bonds.

As used herein, the term “long terminal repeats” or “LTRs” refer to nucleic acid gene sequences which function for integration of dsDNA of a retrovirus into a host chromosome. LTRs also serve as part of the promoter for transcription of viral genes.

As used herein, the term “retroviral packaging element” or “packaging element” refers to a nucleic acid gene sequence, typically near the 5′ end of the RNA-retroviral genome which is involved with packaging viral RNA into virus capsids, for example, a “Psi-sequence” or “packaging signal ψ.”

As used herein, the term “viral particle” or “viral particles” in general refers to a virion or wild-type infectious virus. A viral particle of the present inventions refers to the product of an artificial viral vector assembled within a cell.

As used herein, the term “DNA” or “deoxyribonucleic nucleic acid” refers to a nucleic acid sequence consisting of deoxyribonucleic acids. DNA molecules are said to have “5′ ends” and “3′ ends” because mononucleotides are joined to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. A double-stranded nucleic acid molecule may also be said to have a 5′ and 3′ end, wherein the “5′” refers to the end containing the accepted beginning of the particular region, gene, or structure. A nucleic acid sequence, even if internal to a larger oligonucleotide, may also be said to have 5′ and 3′ ends (these ends are not ‘free’). In such a case, the 5′ and 3′ ends of the internal nucleic acid sequence refer to the 5′ and 3′ ends that said fragment would have were it isolated from the larger oligonucleotide. In either a linear or circular DNA molecule, discrete elements may be referred to as being “upstream” (or 5′) or “downstream” (or 3′) elements. Ends are said to “compatible” if a) they are both blunt or contain complementary single strand extensions (such as that created after digestion with a restriction endonuclease) and b) at least one of the ends contains a 5′ phosphate group. Compatible ends are therefore capable of being ligated by a double stranded DNA ligase (e.g. T4 DNA ligase) under standard conditions.

As used herein, the term “nucleotide” refers to a monomeric unit of nucleic acid (e.g. DNA or RNA) consisting of a sugar moiety (pentose), a phosphate group, and a nitrogenous heterocyclic base. The base is linked to the sugar moiety via the glycosidic carbon (1′ carbon of the pentose) and that combination of base and sugar is called a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence” or “nucleic acid sequence,” and is represented herein by a formula whose left to right orientation is in the conventional direction of 5′-terminus to 3′-terminus.

The term “gene” refers to a nucleic acid (e.g., DNA) sequence that comprises coding sequences necessary for the production of an RNA having a non-coding function (e.g., a ribosomal or transfer RNA), a polypeptide or a precursor. The RNA or polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the sequences located adjacent to the coding region on both the 5′ and 3′ ends for a distance of about 1 kb or more on either end, such that the gene is capable of being transcribed into a full-length mRNA. The sequences that are located 5′ of the coding region and are present on the mRNA are referred to as 5′ untranslated region (UTRs). The sequences which are located 3′ or downstream of the coding region and that are present on the mRNA are referred to as 3′ untranslated regions (UTRs). The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “RNA” or “ribonucleic acid” refers to a nucleic acid sequence consisting of ribonucleic acids.

As used herein, the terms “nucleic acid molecule encoding,” “DNA sequence encoding,” and “DNA encoding,” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “homology”, when in reference to any nucleic acid sequence indicates a percentage of nucleotides in a candidate sequence that are identical with the nucleotides of a corresponding native nucleic acid sequence. Homology may be determined in the latter case by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

As used herein, the terms “subject” and “patient” refer to any organisms including fish, birds, and animals (e.g. livestock, such as pigs, goats, sheep, cows, etc. and zoo animals, such as lions, tigers, bears, etc.) including mammals such as dolphins, dogs, cats, horses and humans.

As used herein, the term “bacteria” refers to any bacterial species including eubacterial and archaebacterial species.

As used herein, the term “adenovirus” refers to a non-enveloped virus having a double-stranded DNA inside of a capsid that in its wild-type form causes respiratory tract infections in humans. When used as a therapeutic expression vector for a recombinant gene, viral replication occurs without integration into the host genome for transient (a few weeks) expression of its transgene.

As used herein, the term “adeno-associated virus (AAV)” refers to a vector that combines some of the advantages of both the retroviral and adenoviral vectors. It is a single stranded DNA parvovirus that integrates into the host genome during replication, thereby producing stable transduction of the target cell. The virus can also infect a wide range of cell types, including both dividing and non-dividing cells. AAV vectors are also not associated with any known human disease and show high efficiency transduction. However they carry a fairly small therapeutic gene insert (around 4.7 kb). They appear to be associated with fewer safety risks than the other viral systems. This is due to the elimination of all sequences coding for viral proteins, thereby greatly reducing the risk of an immune reaction against the vector. There remain, however, the potential problems of insertional mutagenesis and the generation of replication competent virus.

As used herein, the terms “herpes virus” and “herpesvirus” refers to a virus belonging to the Herpesviridae family of large, enveloped double-stranded DNA virus. Exemplary “herpesviruses” include but are not limited to Herpes simplex viruses (HSV-1 and HSV-2), varicella zoster viruses (VSV), Epstein Barr viruses (EBV), and cytomegaloviruses (CMV).

As used herein, the term “respiratory virus” refers to a virus that infects a cell of the respiratory tract (air passages from the nose to the pulmonary alveoli, through the pharynx, larynx, trachea, and bronchi). Exemplary “respiratory viruses” include but are not limited to influenza viruses, parainfluenza viruses, respiratory syncytial viruses (RSV), adenoviruses, rhinoviruses, and severe acute respiratory syndrome (SARS) viruses.

As used herein, the term “kit,” is used in reference to a combination of reagents and other materials.

As used herein, the term “kit” refers to any package or delivery system for packaging or delivering materials. Kits allow for the storage, transport, or delivery of reaction reagents (e.g., cells, buffers, vectors of the present invention) in the appropriate containers) and/or supporting materials (e.g., media, written instructions for performing using the materials, etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain cells for a particular use, while a second container contains selective media. In contrast, a “combined kit” refers to a delivery system containing all of the components of a reaction materials needed for a particular use in a single container (e.g., in a single box housing each of the desired components). The term “kit” includes both fragmented and combined kits.

The term “fluorescent activated cell sorting” or “FACS”, as used herein, refers to a technique for counting, examining, and sorting cells suspended in a stream of fluid. It allows simultaneous multiparametric analysis of the physical and/or chemical characteristics of single cells flowing through an optical and/or electronic detection apparatus. Generally, a beam of light (usually laser light) of a single wavelength is directed onto a hydro-dynamically focused stream of fluid. A number of detectors are aimed at the point where the stream passes through the light beam; one in line with the light beam (Forward Scatter, correlates to cell volume) and several perpendicular to the beam, (Side Scatter, correlates to the inner complexity of the particle and/or surface roughness) and one or more fluorescent detectors. Each suspended particle passing through the beam scatters the light in some way, and fluorescent chemicals found in the particle or attached to the particle may be excited into emitting light at a lower frequency than the light source. By analyzing the combinations of scattered and fluorescent light picked up by the detectors it is then possible to derive information about the physical and chemical structure of each individual particle.

As used herein, the term “transformed cell lines,” refers to cell cultures that have been transformed into continuous cell lines with the characteristics as described above.

Transformed cell lines can be derived directly from tumor tissue and also by in vitro transformation of cells with whole virus, or DNA fragments derived from a transforming virus using vector systems.

The terms “overexpress”, “overexpressing” and grammatical equivalents, as used herein, refer to the production of a gene product at levels that exceed production in normal or control cells. The term “overexpression” or “highly expressed” may be specifically used in reference to levels of mRNA to indicate a higher level of expression than that typically observed in a given tissue in a control or non-transgenic animal.

Levels of mRNA are measured using any of a number of techniques known to those skilled in the art including, but not limited to Northern blot analysis. Appropriate controls are included on the Northern blot to control for differences in the amount of RNA loaded from each tissue analyzed, the amount of 28S rRNA (an abundant RNA transcript present at essentially the same amount in all tissues) present in each sample can be used as a means of normalizing or standardizing the mRNA-specific signal observed on Northern blots. Overexpression may likewise result in elevated levels of proteins encoded by said mRNAs.

As used herein, the terms “culture media,” and “cell culture media,” refers to media that are suitable to support the growth of cells in vitro (i.e., cell cultures). It is not intended that the term be limited to any particular culture medium. For example, it is intended that the definition encompass outgrowth as well as maintenance media. Indeed, it is intended that the teen encompass any culture medium suitable for the growth of the cell cultures of interest.

### DESCRIPTION OF THE DRAWINGS

FIG. 1A-0 show the shRNA expression vectors, based upon pLKO.1-puro, caused Rem super-induction.

FIG. 1A-C shows a schematic vector map of pLKO.1-puro (FIG. 1B), showing an exemplary shRNA insert (FIG. 1A) and locations of additional super-induction sequences: the 5′ long terminal repeat (5′ LTR) contains the Rous sarcoma virus (RSV) U3 region and the HIV-1R and U5 regions, iv (psi packaging sequence), Rev-responsive element (RRE). The human U6 promoter is upstream of the small hairpin (sh) RNA sequence (shown expressed in FIG. 1C). See, also, world-wide web.sigmaaldrich.com/life-science/functional-genomics-and-rnai/shrna/library-information/vector-map.html.

FIGS. 1D&E shows exemplary expression of Renilla luciferase from different shRNA expression vectors. The pLKO.1-control vector stimulated activity from both Renilla (FIG. 1D) and firefly (FIG. 1E) reporter vectors. Cells (293) were co-transfected using calcium phosphate with the empty vector pcDNA3 or 5 μg of pLKO.1c or 5 μg of one of two other pLKO.1 vectors each a different shRNAs against the AAA ATPase p97 (p97/VCP: #4250 and #4252) in the presence or absence of GFP-tagged Rem (12.5 ng) as indicated. In other experiments, the transfection of #4250 showed a measurable decrease in p97 protein levels whereas #4252 did not. Each sample also contained 100 or 250 ng of the Rem-responsive vector pHMRluc and 250 ng of the Rem-non-responsive vector pGL3-control expressing Renilla (FIG. 1D) or firefly (FIG. 1E) luciferase, respectively. Transfections were performed in triplicate with the same total DNA (6 μg) adjusted with pcDNA3. The mean and standard deviation are reported for 100 μg of cell extract relative to the mean for pcDNA3 alone. 100 ug protein, 293 cells, 5 ug LKO vectors and 12.5 ng of GFPRem. A luciferase value of reporter vector co-transfected with an empty vector was set to 1.

FIG. 1F shows exemplary Western blotting results of increased GFPRem expression in the presence of pLKO.1c vector. Extracts from the transfections in FIGS. 1D&E were subjected to Western blotting. Blots were incubated with antibodies specific for GFP (upper panel) or β-actin (lower panel). The positions of uncleaved Rem (GFPRem) and cleaved signal peptide (GFP-SP) are indicated by arrows. 5 ug of LKO vectors and 12.5 ng of Rem expression vector.

FIGS. 1G&H shows exemplary pLKO.1c vector stimulated reporter activity in rat XC cells. Rat XC fibroblasts were transfected using DMRIE-C in triplicate with the indicated plasmids. Samples had a total of 6 μg of DNA in each transfection by adding the pcDNA vector as well as reporters, 250 ng of HMRluc and 250 ng of pGL3-control. Transfections contained the pLKO.1c vector (either 2 or 5 μg) in the presence of 25 ng of GFPRem expression plasmid as indicated. Results are reported as described in FIGS. 1D&E.

FIG. 1I shows exemplary Western blotting of XC cells co-transfected with pLKO.1c that increased GFPRem expression. Extracts from the transfections shown in FIGS. 1G&H were used for Western blotting. Blots were incubated with antibodies specific for GFP (upper panel) or β-actin (lower panel). GFPRem 25 ng.

FIGS. 1J&K shows exemplary stimulated reporter activity by pLKO.1c that did not require the Rem-responsive element. Firefly luciferase is a 61 kDa protein isolated from beetles (Photinus pyralis), while Renilla luciferase is a 36 kDa protein from sea pansy (Renilla reniformis). These enzymes differ in their substrate and cofactor requirements. Transfections in 293 cells using calcium phosphate were performed as indicated in FIGS. 1D&E except that 2.5 ng of control vector pEGFP with a deletion of the EGFP gene (ΔGFP) or GFPRem were added as indicated. Transfections also contained 250 ng of pHMRluc reporter vector (FIG. 1J) or 250 of the same reporter with a deletion of the Rem-responsive element (RmRE). Transfections also contained 250 ng of the pGL3-control plasmid (FIG. 1K).

FIG. 1L Diagram of pLKO.1-based mutant (deletion derivatives of) lentivirus vectors. The 5′ long terminal repeat (light gray box) contains the Rous sarcoma virus (RSV) U3 region and the HIV-1R and U5 regions. The yr (packaging sequence) and Rev-responsive element (RRE) are indicated by boxes. The human U6 promoter is upstream of the control small hairpin (sh) RNA sequence. The human phosphoglycerate kinase (hPGK) promoter is upstream of the puromycin-resistance gene. The 3′ LTR (shorter light gray box) is self-inactivating (SIN) due to a large deletion within the U3 promoter region. The pLKΔHP vector lacks the control shRNA sequence, whereas the pLKΔU6 vector lacks both the U6 promoter and the shRNA sequence. The pLKΔPsi vector has a deletion of the ψ, RRE, U6 promoter and shRNA sequences.

FIGS. 1M&N shows exemplary Luciferase values (FIG. 1M Firefly luciferase and FIG. 1N Renilla luciferase) with Lentivirus vectors lacking shRNAs to stimulate expression of co-transfected genes. The pLKO.1c vectors and deletion derivatives shown in FIG. 1L—were used for transfections of 293 cells. The lentivirus vectors (5 μg each) were co-transfected with 12.5 ng of GFPRem as indicated in the presence of pGL3-control plasmid. Results are reported as in FIGS. 1D&E.

FIG. 1O shows exemplary Western blotting indicating that pLKO—based vectors lacking shRNAs stimulated GFPRem expression. Extracts from the transfection in FIGS. 1M&N were used for Western blots that were incubated with antibodies specific for GFP (upper panel) or β-actin (lower panel). Western blots of extracts expressing GFPRem were scanned and quantitated relative to actin. Results are expressed relative to GFP-SP in the absence of pLKO—based vectors (relative value of 1.0).

FIG. 2A-K show fluorescence microscopy confirmation of stimulation of co-transfected GFP-tagged Rem expression constructs in the presence of lentivirus vectors.

FIG. 2A-F shows exemplary fluorescent microscopy of GFPRem in the presence of lentivirus vectors. Transfections of GFPRem (12.5 ng) into 293 cells were performed in the absence (FIG. 2D-E, lower panels) or presence of 250 ng of the pHMRluc reporter plasmid. Transfections also contained 5 μg of pLKO.1c (FIGS. 2B&E,—middle panels) or pLKΔHP (FIGS. 2C&F-right panels).

FIG. 2G-I shows exemplary fluorescent microscopy of ER-mCherry in the presence of lentivirus vectors. Transfections of the ER-mCherry expression vector (200 ng) into 293 cells were performed in the presence of 5 μg of pLKO.1c (middle panel FIG. 2H) or pLKΔHP (right panel FIG. 21).

FIG. 2J shows exemplary FACS quantitation of EGFP expression in the presence of lentivirus vectors. The average mean fluorescence and range of duplicate transfections of 5 μg of pLKO.1c or pLKΔHP plasmids into 293 cells was determined in the presence or absence of the indicated amount of pEGFP vector relative to the pcDNA control. (FIG. 2K) shows exemplary FACS quantitation of ER-mCherry expression in the presence of lentivirus vectors. The average mean fluorescence and range of duplicate transfections of 5 μg of pLKO.1c or pLKΔHP plasmids into 293 cells was determined in the presence or absence of the indicated amount of pEGFP vector relative to the pcDNA control.

FIG. 3A-F shows that the lentivirus LL3.7 lacking an shRNA insert increases expression of co-transfected genes.

FIG. 3A shows an exemplary schematic diagram of lentivirus constructs. The pLKO.1-control vector labeling is the same as in FIG. 1B. The pLL3.7 vector has a CMV promoter upstream of the 5′ R and U5 region of the HIV-1 LTR. The vector also contains a packaging signal (y), the RRE, a U6 promoter, an internal CMV promoter upstream of the EGFP gene, and the woodchuck hepatitis virus post-transcriptional element (WPRE) 5′ to a second HIV-1R and U5 region.

FIGS. 3B&C shows an exemplary pLL3.7 vector that stimulated co-transfected reporter gene expression (FIG. 3B Firefly luciferase and FIG. 3C Renilla luciferase). Transfections in 293T cells were performed and values are reported as described in FIGS. 1D&E. Transfections included 12.5 ng of GFPRem and either 2 or 5 μg of pLKO.1c or pLL3.7 vector as indicated.

FIG. 3D shows exemplary co-transfection of the pLL3.7 vector that increased GFPRem expression. Extracts from transfections shown in FIGS. 3B&C were used to prepare Western blots for incubation with antibodies specific for GFP (upper panel) or actin (lower panel). The arrows indicate the positions of uncleaved GFPRem precursor or cleaved GFP-SP.

FIGS. 3E&F shows an exemplary pCEP4 vector expressing shRNAs that increased co-transfected reporter genes (FIG. 3E Firefly luciferase and FIG. 3F Renilla luciferase). Transfections into 293 cells were performed and reported as described in FIGS. 1D&E with 12.5 ng of GFPRem and 5 μg of pLKO.1c or pCEP4-based vectors. The shRNAs #1 and 2 (specific for E6AP and HPV18 E6, respectively) were expressed from the H1 promoter.

FIG. 4A-F show that transfer of lentiviral sequences to the pcDNA3 expression vector increases protein expression of co-transfected genes.

FIG. 4A shows an exemplary schematic diagram of pcDNA3 vector containing pLKO.1 sequences. The pcDNA3 vector (pcDNA3psi-sh) contains the CMV promoter upstream of the pLKO.1 sequences from the packaging site through the control shRNA. This plasmid also contains the polyadenylation site from the bovine growth hormone (BGH) gene.

FIGS. 4B&C show exemplary pcDNA3psi-sh construct that stimulated co-transfected reporter gene expression (FIG. 4B Firefly luciferase and FIG. 4C Renilla luciferase). Transfections into 293 cells were performed and reported as described in FIGS. 1D&E. The amounts of transfected vectors were: pLKO.1c or pcDNA3psi-sh (5 μg) and GFPRem (12.5 ng).

FIG. 4D shows an exemplary pcDNA3psi-sh vector that stimulated co-transfected GFPRem expression. Extracts from the experiment shown in FIGS. 4B&C were used for Western blots that were incubated with antibodies specific for GFP.

FIG. 4E shows an exemplary co-transfection of the pLKO.1c vector that did not change the steady-state levels of Renilla luciferase, gapdh or rem mRNA under conditions that increased expression of a gene of interest. PCRs were performed with equal amounts of cDNA reactions performed in the presence or absence of reverse transcriptase or in the absence of added template (water, lane 2). Lanes 3 and 7=pHMRluc; lanes 4 and 8, pLKO.1c (5 ug) and pHMRluc; lanes 5 and 9, pHMR/uc and GFPRem; lanes 6 and 10, pLKO.1c+pHMRluc+GFPRem.

FIG. 4F shows an exemplary Western blotting result of pLKO.1c induced Rem expression. An aliquot of the same transfections used for RNA extractions and RT-PCR in FIG. 4E were subjected to Western blotting and incubation with antibody specific for GFP.

FIG. 5A-F shows co-transfection with gammaretroviral constructs enhances reporter gene expression.

FIG. 5A shows an exemplary schematic diagram of retroviral constructs used for transfections. MigR1 is a gammaretroviral vector that contains the packaging sequence upstream of an internal ribosomal entry site-GFP cassette flanked by mouse stem cell virus (MSCV) LTRs. The MigΔIRES construct has a deletion of the GFP gene and part of the IRES.

FIGS. 5B&C show exemplary MigR1-increased expression of co-transfected reporter genes (FIG. 5B Firefly luciferase and FIG. 5C Renilla luciferase). Transfections in 293 cells were performed and reported as in FIGS. 1D&E. The amounts of vectors used were: pLKO.1c, MigR1, or EGFP (5 μg) and GFPRem (12.5 ng).

FIGS. 5D&E show exemplary MigΔIRES increased expression of co-transfected reporter genes (FIG. 5D Firefly luciferase and FIG. 5E Renilla luciferase). Transfections in 293 cells were performed as in FIGS. 1D&E.

FIG. 5F shows an exemplary MigR1 increased co-transfected GFPRem levels. Extracts from transfections shown in FIGS. 5D&E were used for Western blots that were incubated with antibodies specific for GFP (upper panel) or actin (lower panel). The amounts transfected were: 5 ug MigRI or ΔGFP or pLKO.1c and 12.5 ng of GFPRem (as indicated).

FIG. 6A-C show levels of endogenous proteins are unaffected by transfections with pLKO.1-control.

FIG. 6A show that introduced, but not endogenous genes, show increased protein expression. Exemplary Western blot of unchanged eIF2α phosphorylation in the presence of pLKO.1c. Cells (293) were transfected with the indicated plasmids (12.5 ng of GFPRem and 5 μg of pLKO.1c) for a total of 6 μg. Extracts were subjected to Western blotting and incubated with the indicated antibodies.

FIG. 6B shows an exemplary transfection of pLKO.1c that did not induce the unfolded protein response. Transfections were performed as in FIG. 6A. Extracts were used for Western blotting with antibodies specific for BiP, B23 and GAPDH.

FIG. 6C shows an exemplary transfection of pLKO.1c that did not affect interferon-induced genes. Cells (293T) were transfected as described in FIG. 6A and compared to HeLa or A549 cells treated with β-interferon. Extracts were used for Western blotting with antibody specific for RIG-G.

FIG. 7A-C show the RNA-binding shuttling protein TIA-1 activates expression of Rem-responsive reporter plasmids, but represses the super-induction by pLKO.1c.

FIGS. 7A&B show expression of TIA-1 represses pLKO.1c super-induction of Rem activity. Cells (293) were transfected with pLKO.1c (5 μg) or TIA-1 (either 0.8 or 2.0 μg) in the presence or absence of GFPRem (12.5 ng) (FIG. 7A Firefly luciferase and FIG. 7B Renilla luciferase). Transfections also contained the Rem-responsive or non-responsive vectors (pHMRluc and pGL3-control, respectively). Results are reported as described in FIGS. 1D&E.

FIG. 7C shows Western blotting confirms TIA-1 expression. Extracts from the transfections in FIGS. 7A&B were used for Western blotting and incubation with antibodies specific for GFP or actin (upper and lower panels, respectively).

FIG. 8A-F show that transcription of lentivirus sequences abolishes superinduction of co-transfected genes.

FIG. 8A shows a diagram of bidirectional promoter vectors. The region from the packaging signal (Psi) through the control shRNA in pLKO.1c was inserted into the multiple cloning site of pTRE3G-BI-mCherry.

FIG. 8B shows insertion of the pLKO.1c Psi to shRNA sequence into pTRE3G-BI-mCherry superinduces mCherry in cis. Calcium phosphate transfection of 293 cells was performed in triplicate with 5 μg of the pTRE plasmids and 1 μg of the transactivator as indicated. All cells were incubated with 300 ng/ml DOX at 7 hr post-transfection prior to FACS analysis at 48 hr. The mean and standard deviations of triplicates are reported relative to pTRE3G-BI-mCherry in the absence of transactivator. All values (lanes 2, 3, and 4) were highly statistically significant (p<0.0001) relative to values from lane 1 as was lane 3 relative to 4.

FIG. 8C shows insertion of the pLKO.1c Psi to shRNA sequence into pTRE3G-BI-mCherry superinduces EGFP in trans. Transfections were performed as in panel B except that samples without the pTRE plasmids were included (pcDNA3). All transfections contained 2.5 ng of the EGFP expression plasmid. Without the transactivator, pTRE-Psi-sh elevated GFP expression ˜10-fold compared to pcDNA3 (p<0.0001; lanes 5 and 1) and ˜6-fold compared to pTRE-3G-BI-mCherry (p<0.0001; lanes 5 and 3). Addition of the transactivator in the presence of DOX dramatically reduced GFP expression.

FIG. 8D shows cap-dependent translation increases in the presence of Psi-sh sequences from pLKO.1c. Transfections were performed and reported as in panel C except that 100 ng of the pFR_CrPV_X6 reporter vector was substituted for the EGFP reporter. In the absence of DOX, pTRE-Psi-sh elevated cap-dependent translation ˜13-fold relative to pcDNA3 (lanes 5 and 1; p<0.005) and ˜7-fold relative to pTRE3G-BI-mCherry (lanes 5 and 3; p<0.005).

FIG. 8E shows loss of the 5′ LTR and linearization of pLKO.1c does not affect superinduction. Cells (293) were transfected with 5 μg of the inducer plasmid and 2.5 ng of EGFP expression plasmid and, after 48 hr, analyzed by FACS as described in panel B. Similar levels of superinduction were observed when the 5′ LTR was removed from pLKO.1c or when the plasmid was linearized with enzymes that cleaved within the U3 enhancer or after the shRNA sequence, but was eliminated by multiple cleavages by HinfI. Linearization of pcDNA3 control vector with EcoRI slightly lowered control values. Asterisks indicate statistical significance with control samples containing uncleaved pcDNA3 (p<0.01).

FIG. 8F shows Western blotting confirms that loss of the 5′ LTR and linearization of pLKO.1c does not affect superinduction. A second transfection experiment was performed as described in panel E, but triplicate samples were pooled and used for Western blotting. Relative GFP intensities were quantitated by LI-COR imaging and normalized for actin levels.

FIG. 9A-C shows pLKO.1c expression elevates reporter gene expression in human Jurkat T cells.

FIGS. 9A&B show exemplary expression of pLKO.1 that increased pHMRluc activity (FIG. 9A Firefly luciferase and FIG. 9B Renilla luciferase). Jurkat T cells were electroporated with 1 μg of pHMRluc and pGL3-control in the presence or absence of GFPRem expression vector (0.1 μg). Some samples had 10 or 30 μg of pLKO.1c as indicated. Samples had a total of 42 μg DNA. Results are reported as in FIGS. 1D&E.

FIG. 9C shows exemplary Western blotting of Jurkat cells transfected with pLKO.1c. Extracts from the transfections in panel FIGS. 9A&B were used for Western blots and incubated with antibodies specific for GFP or actin (upper and lower panels, respectively).

FIGS. 10A&B shows the pLKO.1c vector represses both pGL3-control and NFκB-responsive firefly reporter vectors.

FIG. 10A shows exemplary activity of Rem-responsive reporter plasmids increased in the presence of pLKO.1c. The transfections into 293T cells were performed in the presence of one or more reporter genes as indicated under the horizontal line. The co-transfected plasmids were GFPRem (12.5 ng) or pLKO.1c (5 μg) in a total of 6 μg. Results are reported as in FIGS. 1D&E.

FIG. 10B shows exemplary activity of Rem non-responsive plasmids repressed in the absence of pHMRluc. The transfections were performed in the presence or absence of pHMRluc with pGL3-control or NFiB-GL2. The co-transfected plasmids were the same as shown in FIG. 10A.

FIG. 11A-H show stress granules are detectable after transfection.

FIG. 11A-D shows exemplary fluorescent microscopy results of stress granules in the presence of pLKO.1-control or pcDNA3. Cells (293) were transfected with 12.5 ng of GFPRem in the presence or absence of pLKO.1c (5 μg) in a total of 6 μg of DNA per sample. After 48 hr, cells were fixed and permeablized prior to incubation with antibodies specific for G3BP to detect stress granules. Secondary antibody to detect G3BP was labeled with Alexa Flour 594 Nuclei were stained with DAPI. GFP-tagged Rem-SP is observed within nucleoli and detection is increased in the presence of pLKO.1c.

FIG. 11E-H shows exemplary fluorescent microscopy results of stress granules in cells transfected with MigR1 or pLKO-based vectors as described in FIGS. 9A&B. Additional experiments indicated that stress granule formation does not correlate with increased levels of genes detected in the presence of retroviral vectors. Stress granule formation in 293 cells were detected using with anti-G3BP Ab.

FIG. 12 shows a Western blotting reveals that co-transfections with pLKO.1c did not change levels of the stress granule protein G3BP. FIG. 12 shows exemplary transfections performed as described in FIGS. 1D&E. Extracts were used to prepare Western blots that were incubated with antibodies specific for GFP, G3BP or actin.

FIGS. 13A&B shows exemplary schematic diagrams of commercially available DNA Vaccine vectors contemplated for use with the super-induction system of the present inventions.

FIG. 13A shows gWIZ (Genlantis) contains the kanamycin resistance (kanR) gene as a selectable marker and has an intron upstream of the transgene.

FIG. 13B shows pVAX1 (Invitrogen) contains the kanamycin resistance (kanR) gene as a selectable marker. The pUC origin is oriented such that the pUC origin encoded cryptic eukaryotic promoter will transcribe RNA antisense to the transgene. See, Williams, “Vector Design for Improved DNA Vaccine Efficacy, Safety and Production.” Vaccines, 1:225-249 (2013), herein incorporated by reference in its entirety.

FIGS. 14A&B shows exemplary VA-RNA structural RNA contemplated for use in the present inventions for super-induction. FIG. 14A shows VA-RNA in pMT2 vector [SEQ ID NO: 20] and FIG. 14B shows VA-RNA in Ad serotype 5 (Kamel, et al. 2013) [SEQ ID NO: 21].

FIG. 15 shows exemplary super-induction of GFP by VA-RNA after co-transfection (and expression) of 2.5 ng of an EGFP expression plasmid. pcDNA3 control, pLKO.1c super-induction plasmid, pMT2 adenoviral gene containing plasmid on a pBR322 backbone, see FIG. 16, pMT2deltaVA mutant without the majority of the VA sequences, see FIG. 17.

FIG. 16 show exemplary schematic of a pMT2 adenoviral gene containing plasmid derived from pBR322. See, a VA-RNA sequence expressed from a VA-RNA encoding DNA sequence inserted into the pMT2 plasmid as Ad VA1, i.e. black arrow at the bottom of the figure. (Adgene; Kaufman et al., MCB 9(3):946-958, 1989).

FIG. 17 show exemplary schematic diagram of a pMT2deltaVA mutant plasmid. This plasmid is without the majority of the VA-RNA sequence as shown the in the pMT2 plasmid (FIG. 16). See, VA-RNA leftover (partial) sequences annotated at the bottom of the figure. (Adgene; Kaufman et al., MCB 9(3):946-958, 1989).

FIG. 18A-C shows exemplary VA-RNA alignments and consensus sequences that may find use as highly structured RNA of the present inventions (Ma and Mathews, J of Virology, 70(8):5083-5099 (1996).

FIG. 18A shows sequence alignment of VA-RNA gene regions. Human adenovirus sequences and SA7 are shown, except that group D is represented by the four most divergent sequences (Ad10, Ad32, Ad8, and Ad9) and a consensus sequence (D-con) for group D viruses (SEQ ID NOs:22-45). Initiation and termination sites of the VA RNAs and the splice sites of the terminal protein precursor and 52,55K protein mRNAs are indicated. The regions to which the PCR primers and the internal sequencing primers hybridize are marked with arrows and boxed.

FIG. 18B shows structural features in VA RNA sequences. Alignments of prototype VA RNAs (also see Table 3 in Ma and Mathews, J of Virology, 70(8):5083-5099 (1996)) generated with the PILEUP program were arranged into three superfamilies, separated by blank lines (SEQ ID NOs:46-84). The serotype groups to which these RNAs belong are labeled on the left. The conserved tetranucleotide sequences, GGGU and ACCC, are boxed and shaded. Regions which can pair and form the duplex structures of the apical stem (AS) and terminal stem (TS) are boxed.

FIG. 18C shows sequences of VA RNA prototypes (also see Table 3 in Ma and Mathews, J of Virology, 70(8):5083-5099 (1996)) were aligned with the PILEUP program and manually optimized for sequence conservation (SEQ ID NOs:85-143). The consensus sequence for each family is shown below the prototype VA-RNA sequences. The consensus sequences for the eight families are arranged to generate a general consensus (Consen) and three superfamily consensus sequences at the bottom. Numbers shown on the line above the general consensus sequence represent the number of occurrences of each consensus nucleotide in the eight family consensus sequences. Asterisks and letters two lines above the general consensus sequence indicate the positions of the A box, GGGU sequence, B box, and ACCC sequence. Nucleotides that do not match the consensus are shown in lowercase letters. Nucleotides that match the general consensus are represented by dashes in individual VA RNA sequences and are capitalized in the family consensus sequences. Gaps are represented by dots. The code used to represent degenerate nucleotides is as follows: M for A or C; R for A or G; W for A or U; S for C or G; Y for C or U; K for G or U; V for A, C, or G; H for A, C, or U; D for A, G, or U; B for C, G, or U; and N for A, C, G, or U.

### DESCRIPTION OF THE INVENTION

The present invention relates to methods, compositions and kits for increasing expression of transfected genes through the use of super-induction nucleic acid sequences, including highly structured RNA. In particular, co-transfection of super-induction nucleic acid sequences including, for example, shRNA encoding sequences, retroviral elements, and (in other embodiments) VA-RNA encoding sequences, is contemplated for enhancing the expression of co-transfected transgenes in a cell. More specifically, the super-induction sequences are contemplated for use, in one embodiment, with mammalian expression plasmids and gene therapy vectors for increasing transgenic protein activity levels in cells and tissues. In one embodiment, the super-induction sequences of the present inventions are contemplated for use with known DNA vaccination vectors for enhancing therapy or enhancing protective immunity.

Currently, several problems are associated with biologically active transgene expression in host cells, in particular for mammalian cells. Specifically, the use of current vectors for genetic therapy show low expression, particularly in primary transfected cells and in terminally differentiated cells. Further, merely a few transformed cell lines show optimal DNA uptake and protein expression from viral or plasmid vectors. Therefore, the majority of genes in current vectors are inefficiently expressed in primary cells, transformed cell lines or in highly specialized cell types (terminally differentiated). Successful gene therapy should include the sustained expression of the transgene even in primary or highly specialized cell types. Further, current vectors used for molecular biology or gene therapy have an upper boundary for gene length within the vector.

The inventors contemplate improvements in gene therapy and increase of effectiveness of gene therapy, including DNA vaccines, using super-induction sequences described herein. Advantages include but are not limited to constructs and methods that will work in multiple cell types and using a variety of transfection methods as super-induction success appears to be independent of the transfection method. Specifically, the super-induction sequences improved introduced gene expression by at least 2-fold, and often from 5-to-20-fold, or more, without additional cloning steps. Increased protein expression is apparently independent of the type or length of gene transferred. While the elements can be cloned into other vectors containing the gene of interest, this is not necessary. Instead, the super-induction elements can simply be co-transfected with the vector containing the gene of interest. This should permit the use of lower amounts of vectors for gene therapy. Use of lower amounts of vectors should prevent adverse immunological responses or the development of cancer from random retroviral integrations into host genomes. The inventors contemplate that multiple sequences described herein will be combined to optimize gene expression for different applications in therapeutic and preventative medicine.

Further, based on experiments described herein, certain super-induction elements are contemplated to improve protein expression in mammalian cells for gene therapy and DNA vaccines, etc. by inclusion of such elements in existing vectors comprising a gene of interest. While not necessary, if desired, such elements can be cloned into the existing vector.

Specifically, described herein is the unexpected observation that co-transfection of certain retrovirus-based vectors led to significantly (2-20+fold) increased levels of proteins encoded by co-transfected expression plasmids, i.e. super-induction. During evaluation of this result, the inventors discovered that low induction of expression levels of a co-transfected genes caused by certain retroviral sequences, was augmented by inclusion of sequences corresponding to small hairpin (sh) RNAs. Further, the inventors discovered that co-transfection of an adenoviral vector expressing an adenovirus VAI-RNA also showed super-induction for expression of co-transfected eGFP. This effect was not provided by a co-transfected adenoviral vector having a partial deletion of the VAI-RNA sequence. Thus, in one embodiment, the introduction of certain retrovirus vectors or plasmid vectors expressing super-induction sequences is contemplated to increase expression of genes from other vectors (in trans) when introduced at the same time into a host (co-transfection).

Increased expression can also be achieved by cloning in, if desired, retroviral elements such that they can be cis-acting elements. Again, such elements are retroviral sequences and preferably include combinations of highly structured RNAs (i.e. shRNAs). It is not necessary that a highly structured RNA recognize a mammalian sequence. In fact, it is preferable if the highly structured RNA does not. These vectors which include super-induction sequences, such as specific lentivirus sequences and a structured RNA sequence, in particular a shRNA, a VAI-RNA, a VAII-RNA, and the like, increased protein levels and activity above that obtained from current mammalian expression plasmids and gene therapy vectors. In one contemplated embodiment, a promoter in operable combination with a VAI-RNA would provide super-induction as a cis-acting element. In one contemplated embodiment, a promoter in operable combination with a VAII-RNA would provide super-induction as a cis-acting element.

One unexpected feature of the present inventions is that sequences corresponding to shRNAs increased expression of genes in trans. This is unexpected because shRNAs are generally used to REDUCE expression of specific genes by knocking down such genes, see for example, Bridge, et al., “Induction of an interferon response by RNAi vectors in mammalian cells.” Nature Genetics 34:263 (2003), describing RNA vectors that express short hairpin RNAs (shRNAs) from RNA polymerase III (Pol III) promoters as a promising new tool to reduce gene expression in mammalian cells. In contrast, experiments shown herein demonstrated that any one of several shRNA encoding sequences in lentiviral vectors increased expression of genes present on co-transfected vectors. Thus, transgene expression was increased without recloning of the original vector. Thus in one embodiment, a method of super-induction is provided for increased expression of a transgene without recloning to a different vector.

Another advantage of using a super-induction vector of the present inventions is that inclusion of this vector would allow introduction of less DNA for transfection or gene therapy purposes or allow higher translation of a co-introduced gene from a plasmid vector. Thus reducing adverse side effects resulting from the use of large amounts of current therapeutic retroviral vectors.

In particular, the retroviral sequences assisting with super-induction were identified by the deletion of highly structured RNA regions from retroviral vectors. This led to the finding that shRNA, along with a U6 promoter region, 5′ UTR, RRE and psi packaging regions, were sufficient to provide super-induction. Therefore, RNA structures within retroviral genomes and without retroviral genomes were shown herein to facilitate in trans the translation of other RNAs leading to increased protein expression from the introduced gene (but not the endogenous genes of the cell). Thus in any case where one is doing gene transfer, one can optimize translation and expression with this co-transfection system including super-induction sequences.

I. Lentivirus and Gammaretroviral Vectors Comprising shRNA Increase Expression of Co-Transfected Transgenes.

During the development of the present inventions, lentivirus vectors (for example derived from HIV-1) and gamma retroviral vectors (for example, derived from murine leukemia virus) were found to increase the expression of co-transfected genes. However, with the addition of a highly structured RNA, such as a shRNA, there was an even higher and unexpected increase in expression of co-transfected genes without a corresponding increase in the expression of endogenous proteins in this system. In particular, although increased levels of protein from co-transfected genes were observed, a corresponding increase in RNA levels of endogenous genes was not observed. Elevated protein expression was found to be independent of the unfolded protein response and eIF2α phosphorylation. For comparison, expression of the RNA-binding protein TIA-1, a protein that shuttles between the nucleus and cytoplasm, but not G3BP, increased the activity of specific reporter constructs, but reduced expression from co-transfected lentivirus vectors. Elevated protein expression from super-induction was also associated with a lower stress response of cells after transfection. Therefore, HIV-1 and other retrovirus constructs having highly structured RNA regions, i.e. shRNA, induced an increase in expression of co-transfected genes in trans by an eIF2α-independent mechanism. The inventors contemplate using this unexpected discovery for the design of more effective gene therapy and other applications including modulation of the stress response by transfection of therapeutic retroviral vectors.

A. Lentiviral shRNA Expression Vectors Unexpectedly Caused Super-Induction of Rem Activity and Increased Rem Protein Expression.

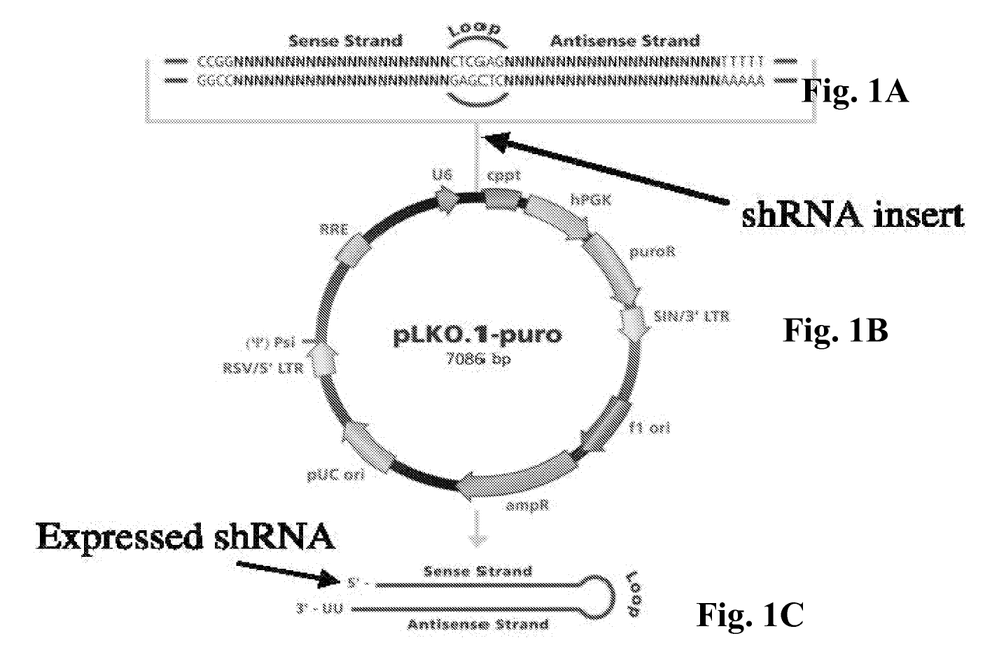

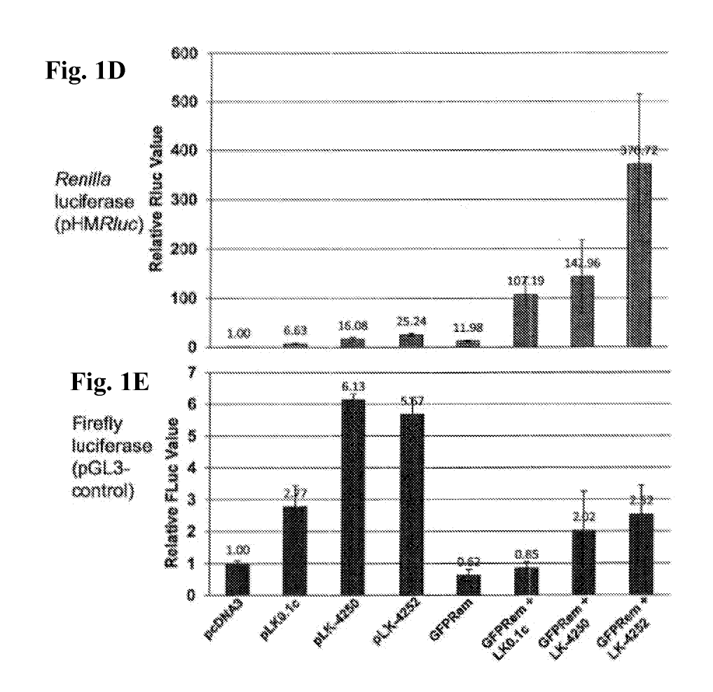

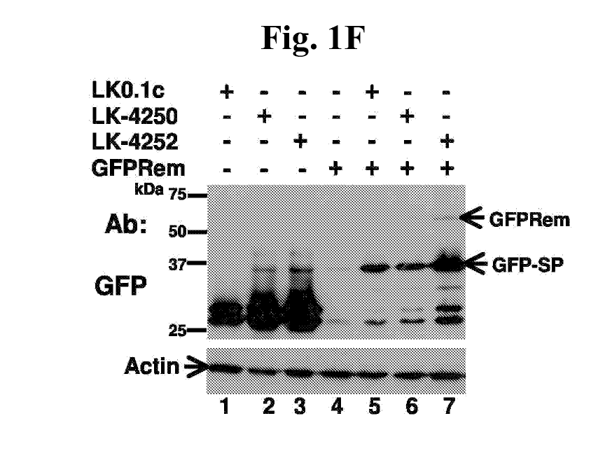

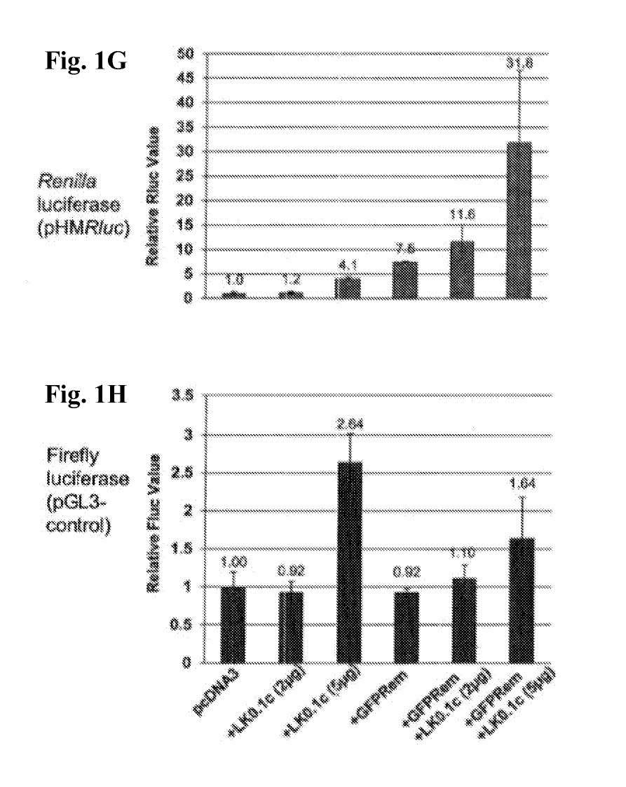

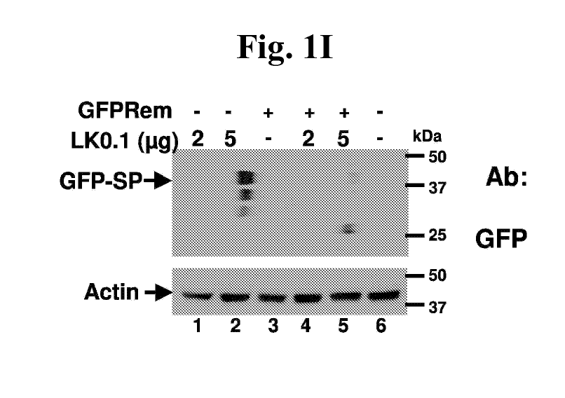

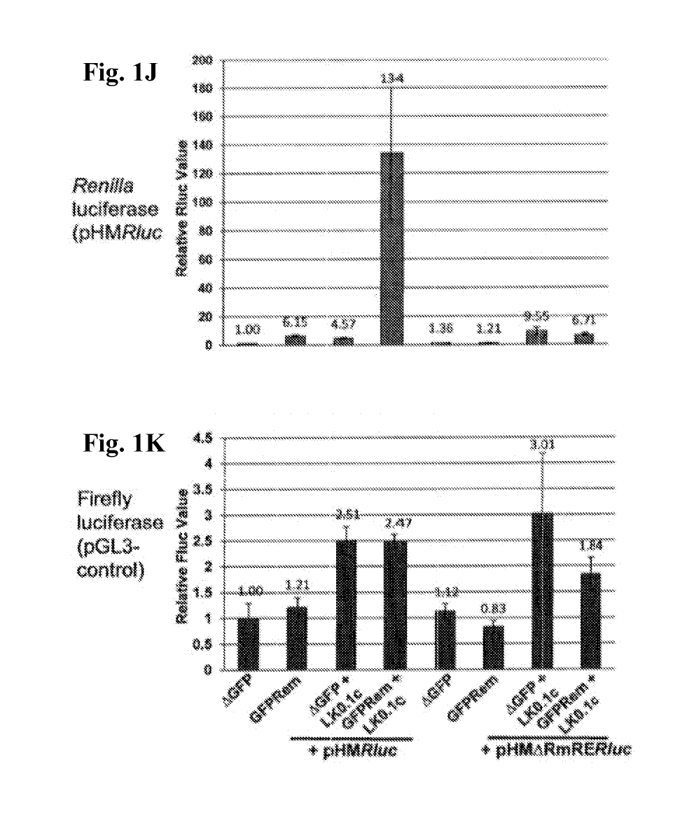

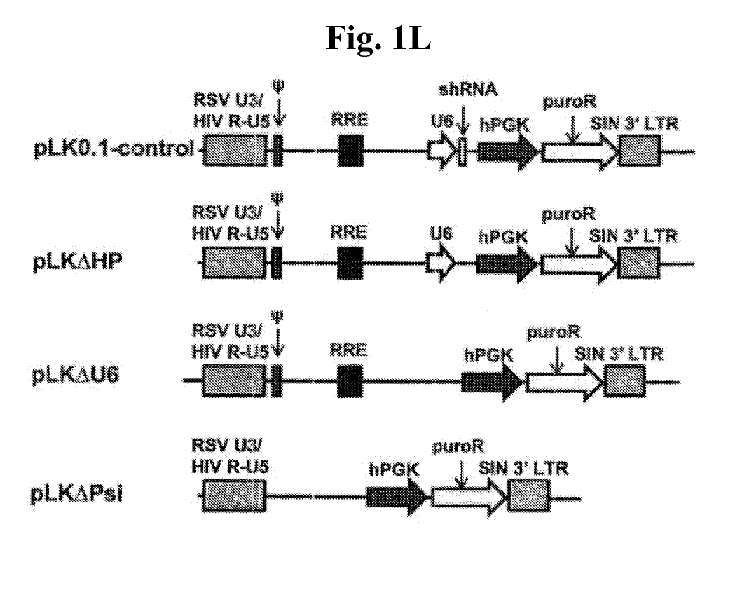

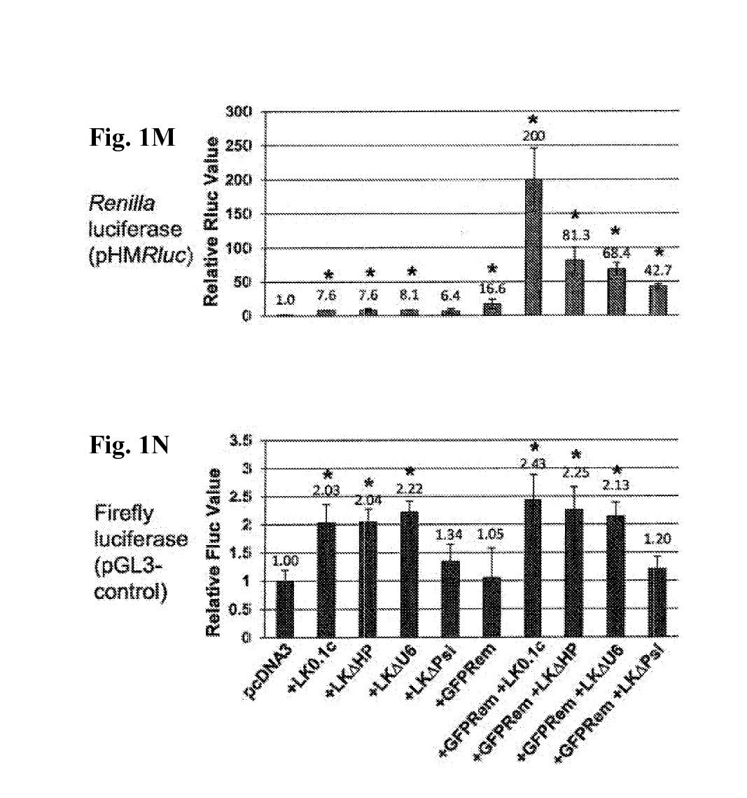

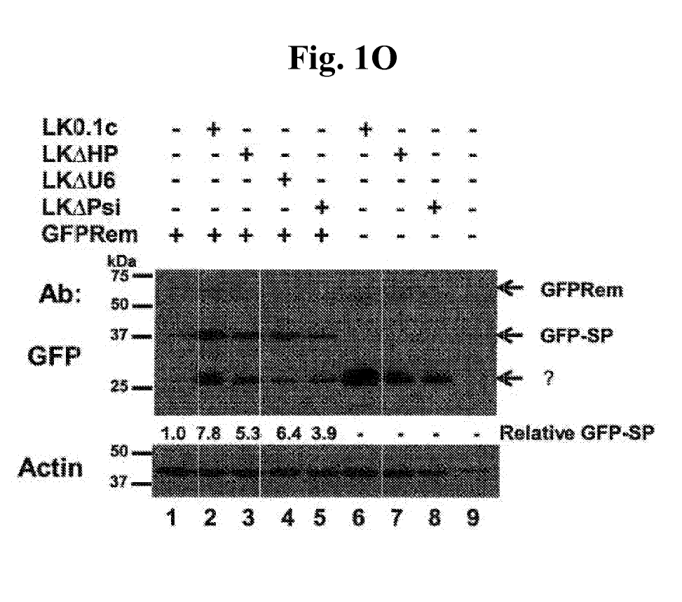

During experiments involving co-transfection of constructs expressing GFPRem plus two gene silencing plasmid constructs and a control pLKO plasmid, (see, for example, pLKO.1 control puroR in FIG. 1B, the inventors observed surprising super-induction results instead, FIG. 1D. For comparison, a luciferase value of reporter vector co-transfected with an empty vector was set to 1. Of the pLKO.1 based plasmids, #4250 and #4252 contained a different inserted shRNA sequence to target cellular protein p97/VCP in order to knock down (silence) p97/VCP activity while the control pLKO plasmid contained an shRNA with no known mammalian gene target. Although in other gene silencing assays, transfection with #4250 showed a measurable decrease in p97 protein levels in comparison to #4252 and pLKO.1-puro control, the super-induction appeared to not rely upon a shRNA having gene silencing activity. Thus in one embodiment, a shRNA preferably does not have a known target gene.

For context, results from the inventors' experiments using dominant-negative p97 expression constructs indicated that Rem SP function of MMTV was dependent on wild-type p97 protein. Therefore, after the knock down of p97/VCP by the siRNA derived from an expression vector comprising a shRNA encoding nucleic acid sequence (#4250), the inventors' expected to observe a corresponding decrease of Rem activity. In contrast, at least three unrelated shRNA sequences expressed from a vector having a pLKO.1 backbone, including a pLKO.1 control vector containing non-human and mouse specific shRNA sequences, caused super-induction of Rem activity. Thus, Rem super-induction was apparently caused by the shRNA vector itself and was not a specific effect of p97/VCP gene knock-down by a specific siRNA.

1. LKO.1 shRNA Plasmids Caused Super-Induction of Co-Transfected Genes in Human Kidney Epithelial Cells.

Because this result was unexpected, the inventors designed several experiments for exploring the capability of the pLKO.1 vector and then identified specific vector sequences responsible for this super-induction. Two reporter vectors were initially used that were capable of expressing luciferase, pHMRluc (containing a Rem responsive Renilla luciferase gene and a RmRE sequence) and a pGL3-control (firefly luciferase without a Rem-responsive sequence). The pGL3-control firefly luciferase was used as a control to normalize the results for differences in transfection and expression efficiency between these reporter plasmids. The pGL3-control and pHMRluc reporters were elevated approximately 2-to-6-fold in the presence of pLKO.1c, while the other two pLKO.1 constructs showed further increases in firefly luciferase. In contrast, when the pLKO.1 vector (with any of the 3 shRNA inserts) was co-transfected with the pHMRluc plasmid and a Rem expression vector, Renilla luciferase activity was increased approximately 10- to 30-fold while firefly luciferase was not increased to these levels. (FIGS. 1D&E). Western blotting of transfected cell extracts verified that Rem expression levels were also highly induced as Rem production increased approximately 5-fold (FIG. 1F. The same super-induction effect of pLKO.1 was observed in co-transfections with Rev and Rex in place of Rem.

2. LKO.1 shRNA plasmids caused super-induction of co-transfected genes in mammalian cells.

Although the effect was smaller, pLKO.1c also increased expression of pHMRluc in the presence or absence of the RmRE in XC rat fibroblast cells transfected by a lipid-based method (FIGS. 1G&H) and increased Rem levels (FIG. 1I).

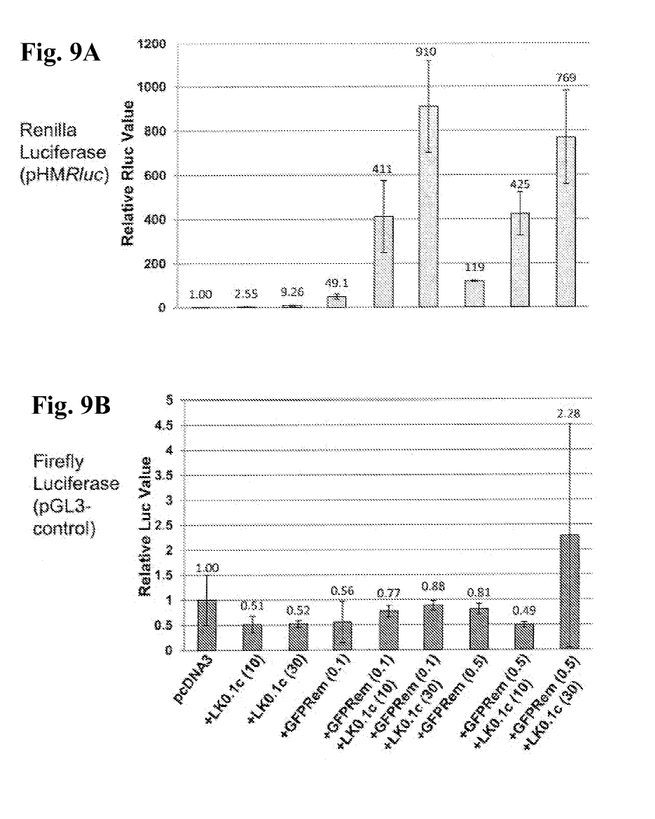

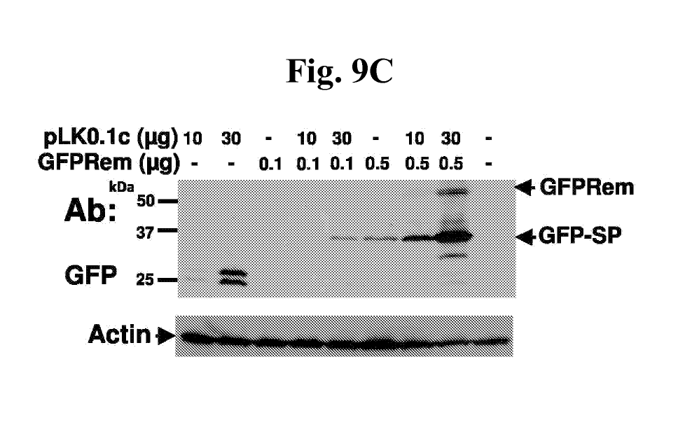

Jurkat human T cells were co-transfected by electroporation with similar results (FIG. 9A-C), indicating that pLKO.1c super-induction is not cell-type, species or method specific. The increase in pHMRluc activity observed after co-transfection with pLKO.1c was independent of the presence of the Rem-responsive element (RmRE), but the induction was dependent on Rem (FIGS. 1J&K). Samples received the same total amount of DNA (6 μg), indicating that different DNA levels were not responsible for changes in transfection efficiency and protein expression. Therefore, the higher induction in the presence of Rem was likely due to stimulation of both basal Rem levels and Renilla luciferase values.

This super-induction effect was observed in human kidney epithelial cells (293 and 293T) as well as transformed rat fibroblasts and transformed human T cells. Since different transfection methods were used (electroporation, calcium phosphate, and lipid reagents) for these cell lines, introduction of the transfected DNA through a specific technique was not required. These experiments indicated that the effect of pLKO.1c occurred in trans, was not limited to specific cell types or species, and was not related to a type I interferon response.

3. LKO.1 shRNA Plasmids Did not Induce Endogenous Proteins.

Total protein levels were examined by Coomassie staining and by incubation of Western blots with antibody specific for endogenous actin (FIG. 1F). However, there were no detectable differences in endogenous protein expression. A similar result was obtained if pLKO.1c expressed a control shRNA or an shRNA specific for the AAA ATPase, p97, although 2-to-3-fold differences were observed (FIG. 1F). These results suggested that the pLKO.1c vector increased expression from exogenously expressed genes in trans but not expression of endogenous genes.

4. Co-transfection effects of other lentiviral shRNA expression vectors.

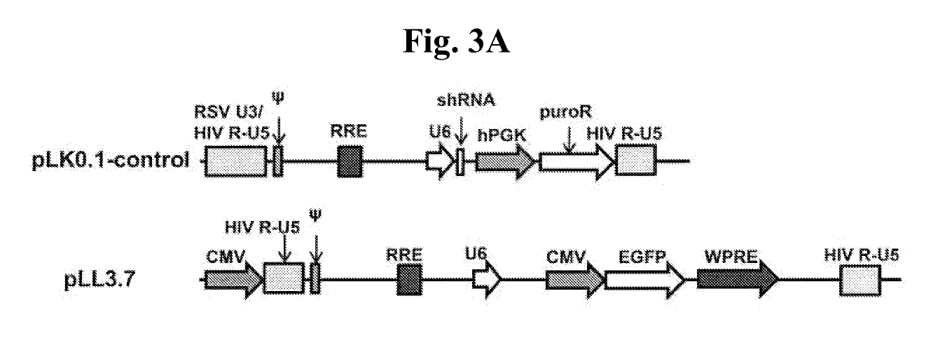

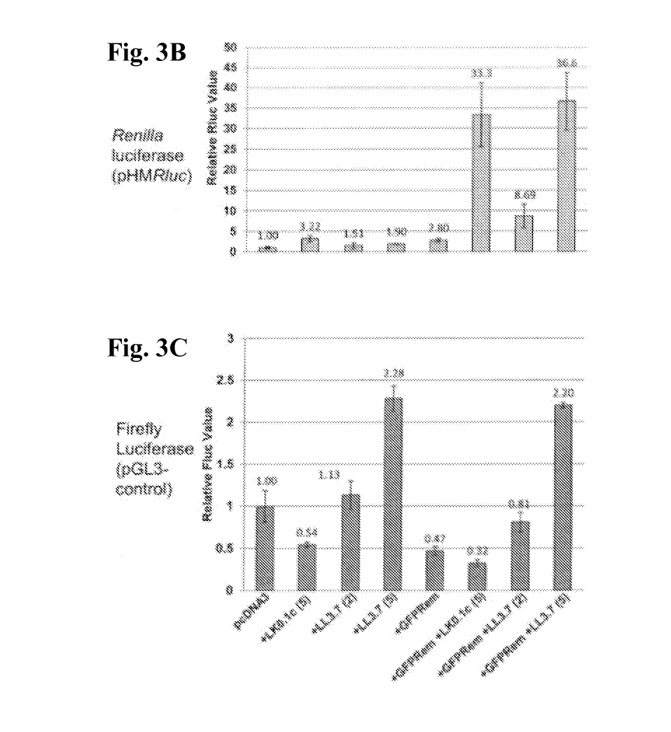

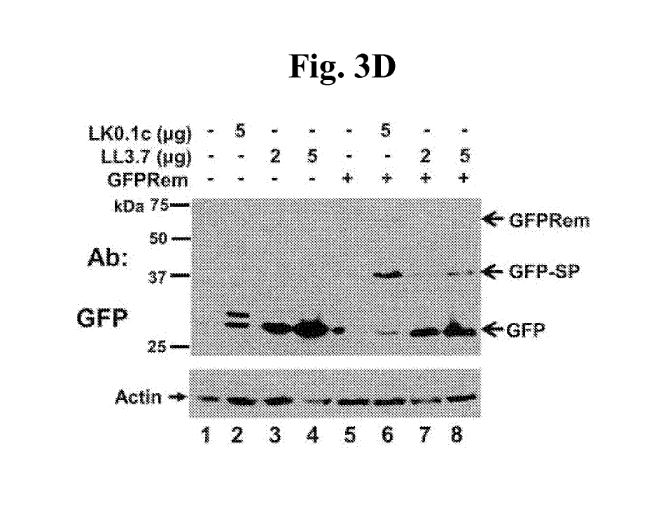

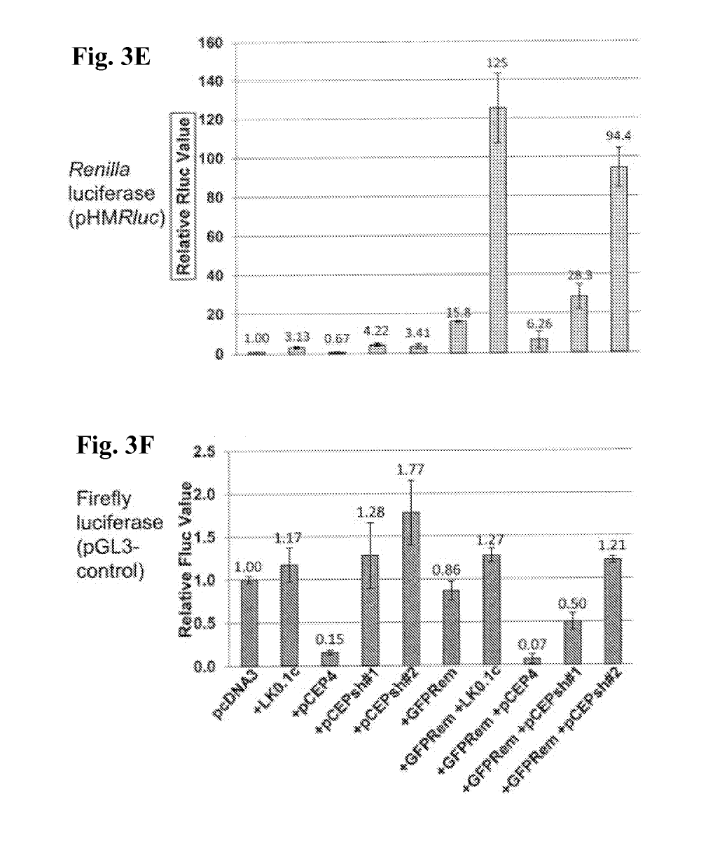

To determine if protein expression was enhanced by other lentiviral shRNA expression vectors, 293T cells were co-transfected with Rem reporter and expression vectors in the presence of pLKO.1c or pLL3.7 (FIG. 3A). The pLL3.7 vector induced basal Renilla luciferase activity from pHMRluc up to 13-fold in the absence of Rem (FIGS. 3B&C), whereas pLKO.1c induced Renilla activity in the presence of Rem. Compared to pLKO.1c, pLL3.7 had little effect on GFP-SP expression by Western blotting (FIG. 3D). Co-transfection of the pLL3.7 vector also increased activity from the pGL3-control vector up to approximately 2-fold. Thus, although the shRNA hairpin was a major determinant of increased expression in the presence of pLKO.1, other sequences in the lentiviral vector influenced expression from co-transfected vectors.

5. Effects of shRNA Expression in the Absence of Lentiviral Sequence.

LKO.1 is a lentiviral vector containing part of the HIV genome, including the 5′ LTR, 5′ UTR, the 5′-end of the gag gene, the RRE, part of the env gene and the 3′ LTR. To test the effect of shRNA expression in the absence of lentiviral sequences, Rem expression and reporter vectors were co-transfected together with a non-lentiviral shRNA expression vector. pCEP4 (Invitrogen) is an episomal mammalian expression vector that does not contain lentiviral elements.

An shRNA vector based on pCEP4 was developed by inserting a histone H1 polymerase III promoter followed by a hairpin into the pCEP4 vector. The pCEP4 control vector and two of the pCEP4-based hairpin-containing vectors, E6AP-3 and HPV18E6 (kindly provided by the Huibregtse laboratory), were co-transfected with GFPRem expression plasmids into 293 cells. pLKO.1 was independently co-transfected with GFPRem as a control. The normalized luciferase activity values showed that none of the pCEP4-based vectors induced additional Rem activity, whereas pLKO.1 caused a 4-fold increase. Surprisingly, pCEP4 and E6AP-3 decreased both firefly and Renilla lucifierase values significantly in the absence and in the presence of Rem. However, the HPV18E6 hairpin-containing vector did not decrease firefly values.

Unlike pLKO.1, pCEP4 slightly decreased Renilla reporter expression in the presence or absence of Rem with larger (up to 10-fold) decrease of firefly reporter activity, which lacks the Rem-responsive element (FIGS. 3E&F). pCEP4 vectors were tested expressing two different shRNAs expressed from the H1 promoter. The pCEP4 vector carrying either shRNA gave 3-to-4-fold increases in Renilla luciferase levels in the absence of Rem. The pCEP4 vector carrying shRNA#1 specific for E6AP had less than a 2-fold effect on Rem-induced expression, whereas shRNA#2 specific for HPV18E6 (not present in these cells) gave approximately 6-fold super-induction in the presence of Rem, comparable to that observed with pLKO.1c, which expresses a control shRNA.

Together, these experiments showed that sequences derived from shRNAs as well as lentiviruses contribute to maximal induction of co-expressed genes by pLKO.1c.

B. Co-Transfection of a Gammaretroviral Vector as a Trans-Activator without shRNA Increased Exogenous Protein Expression.

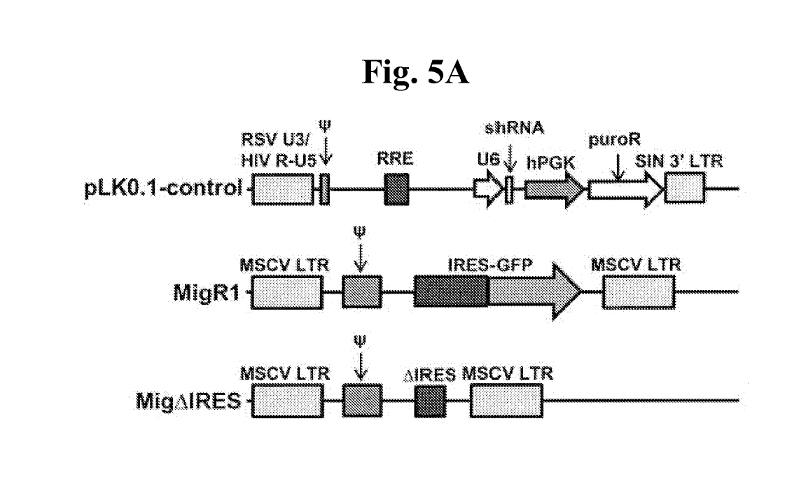

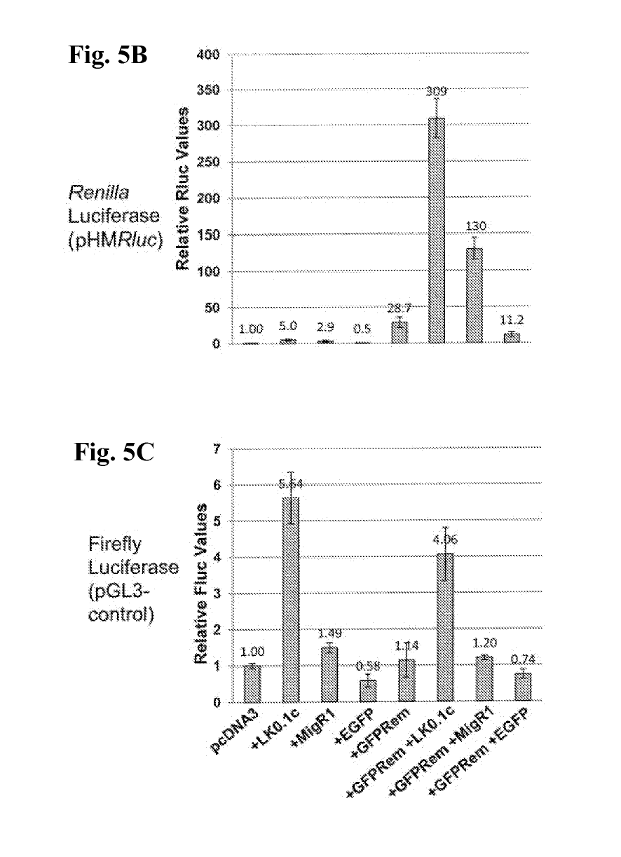

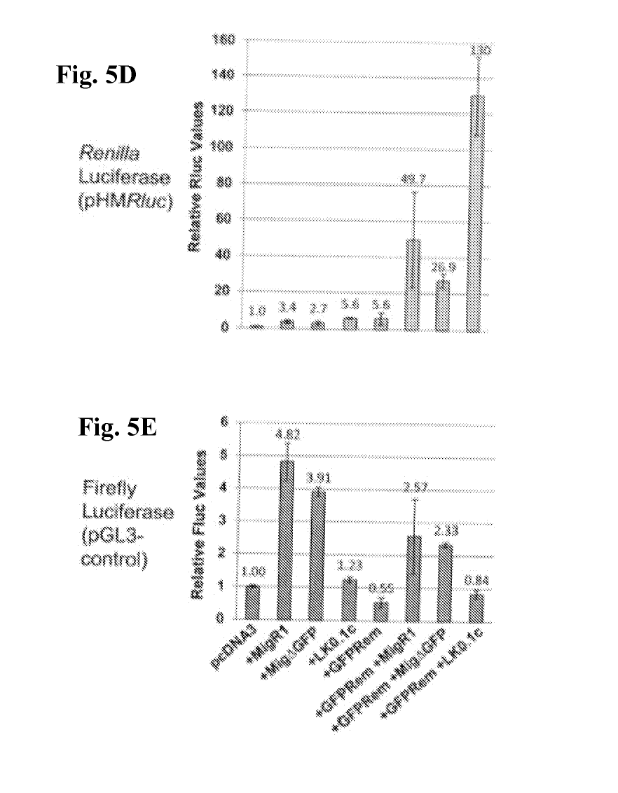

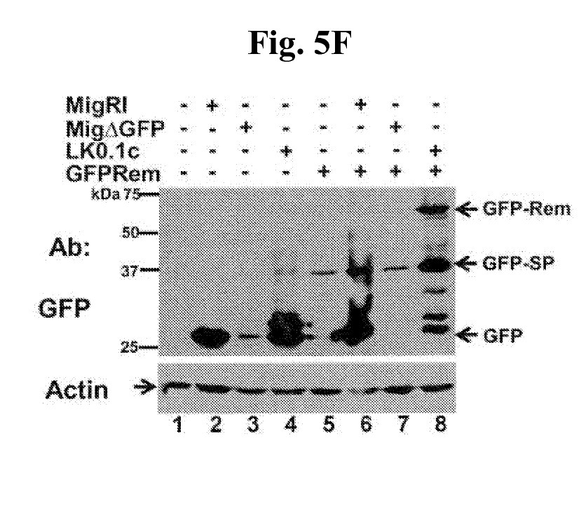

Co-transfection of the gammaretroviral vector MigR1 also stimulated gene expression in trans. Because the use of lentiviral sequences, described herein, that resulted in super-induction and because the lentiviral sequences are functionally similar to those in other retroviruses, a gammaretroviral vector MigR1 (FIG. 5A) containing a psi packaging element and an IRES-GFP element, where IRES is considered another highly structured RNA was tested for super-induction. Thus, Addgene plasmid 27490: https://www.addgene.org/search/advanced/?q=migR1), described in Pear, et al., Blood. 92(10):3780-92 1998, derived from MSCV (Murine Stem Cell Virus), was co-transfected together with a Rem expression plasmid and reporter vectors in both 293 and 293T cells. In 293 cells, the firefly luciferase value from the co-transfected pGL3-control vector was induced 5.6-fold by pLKO.1c, however MigR1 slightly increased reporter values (1.5-fold) (FIGS. 5B&C). In comparison, transfection of pLKO.1c resulted in a 5-fold increase in Renilla luciferase activity in the absence of Rem, whereas MigR1 gave an approximately 3-fold increase. For comparison, MigΔIRES, a deletion mutant of the IRES-GFP sequence from MigR1, showed a lower effect on induction of gene expression in trans (FIGS. 5D&E).

In contrast, inclusion of the plasmid-based vector, pEGFP-N3 (Clontech), decreased both Renilla and firefly luciferase activities. Rem expression induced Renilla reporter values approximately 29-fold and was further induced approximately 11-fold by the addition of pLKO.1c and 4.5-fold by MigR1. Western blotting confirmed that Rem, but not actin, expression increased in the presence of pLKO.1c (FIG. 5F). A similar trend was observed in 293T cells with the two retroviral vectors inducing Renilla luciferase, whereas the plasmid-based vector was inhibitory.

Therefore, gamma retroviral sequences, similar to the lentiviral sequences, caused an increase in expression of co-transfected genes, although not at the same levels observed using the shRNA lentiviral plasmids. These results support the role of the shRNA in retroviruses as highly structured RNA regions that are altering the translation of co-expressed genes whereas IRES, considered a highly structured RNA, did not.

C. Mutational analysis shows the contribution of the shRNA sequence to the super-induction effect.

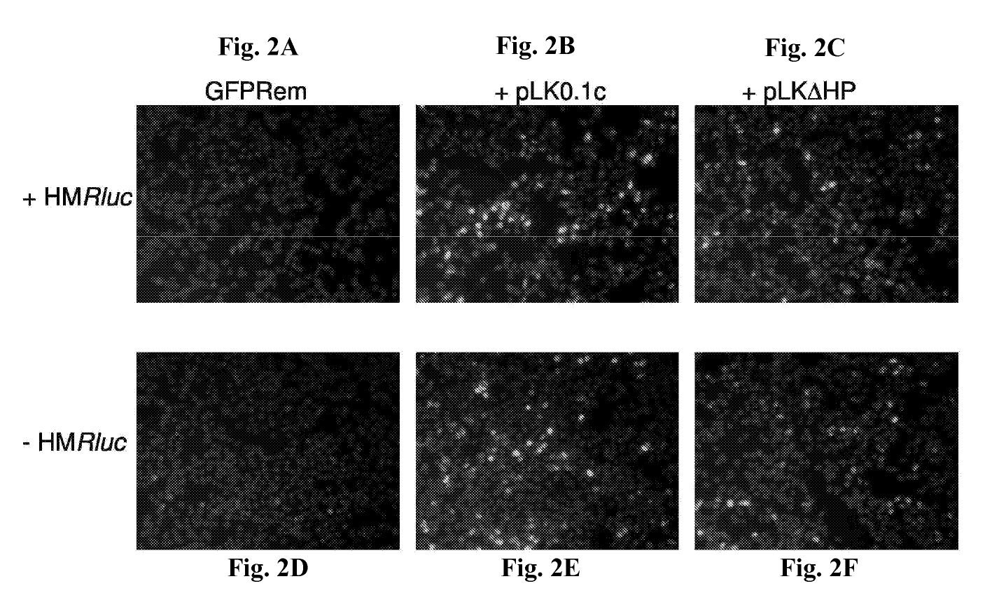

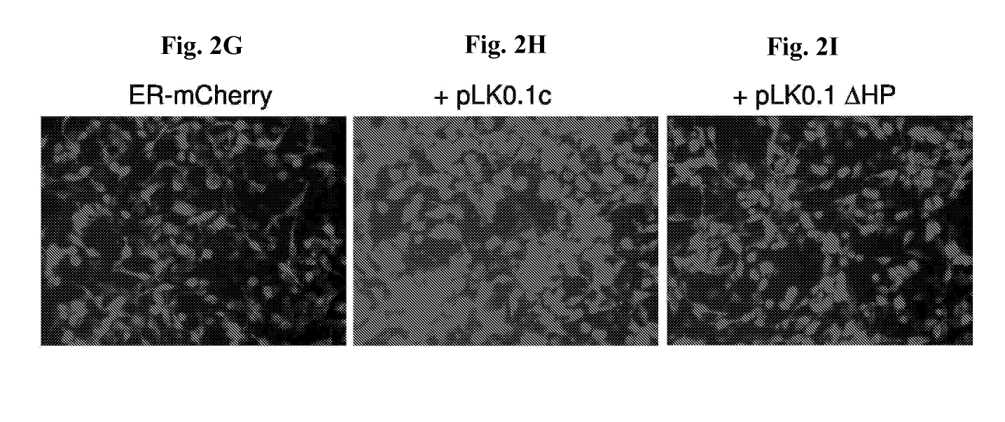

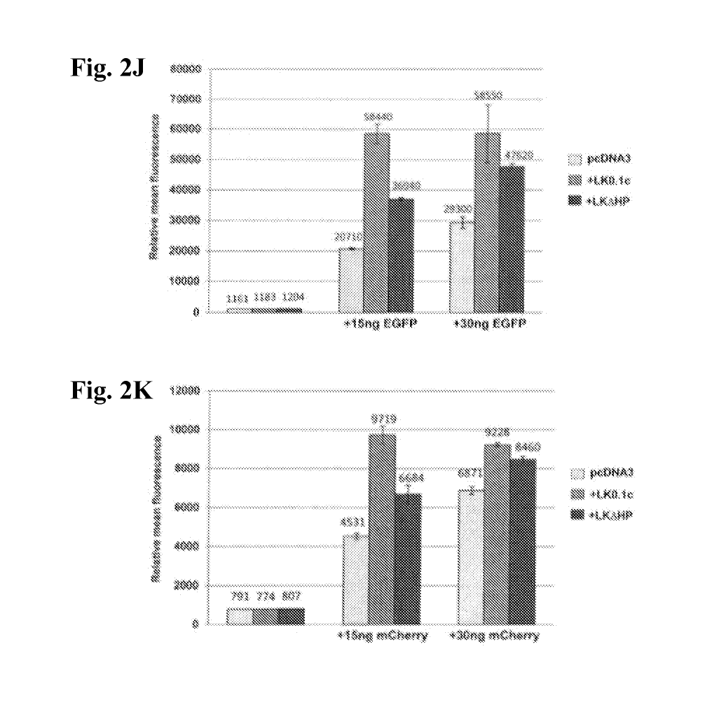

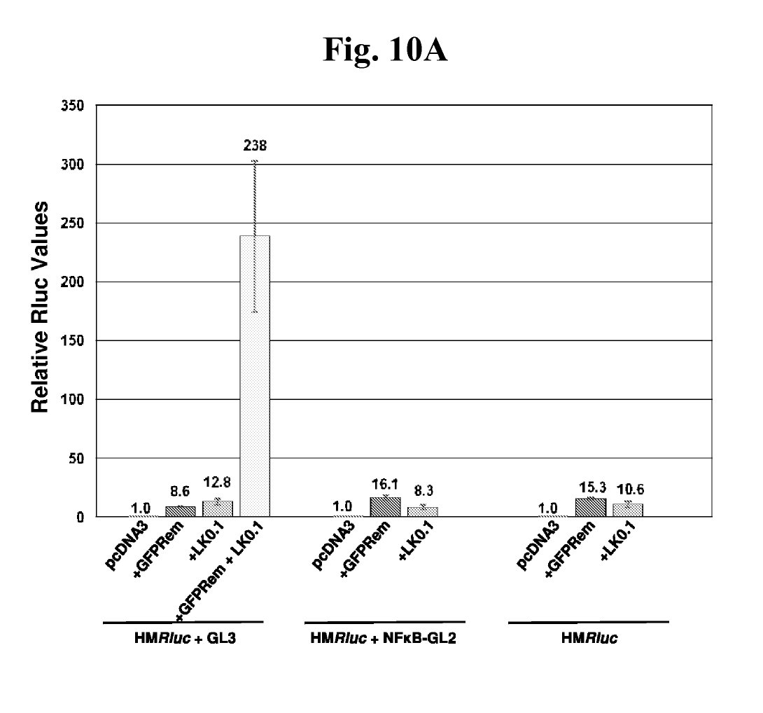

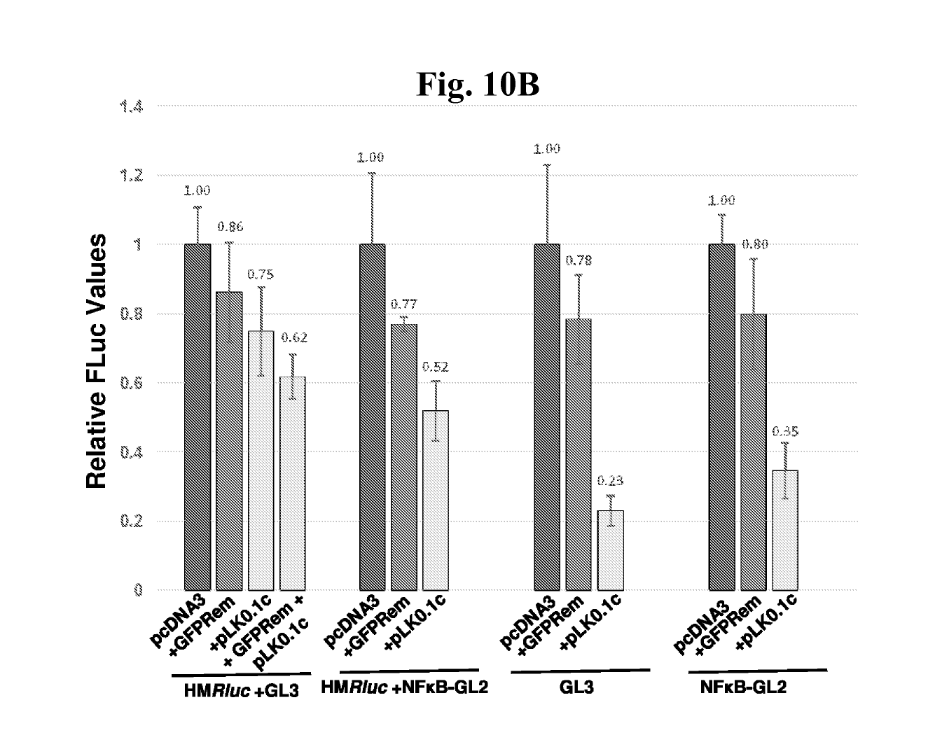

Mutation of the vectors was performed to test each type of sequence in the pLKO.1 plasmid (FIG. 1L). The contribution of the pLKO.1 hairpin to the super-induction effect was greater than the retroviral sequences alone. In one mutant pLKO.1 plasmid the hairpin RNA sequence was deleted, resulting in a vector labeled LKΔHP. Surprisingly, the deletion of the hairpin led to a lower super-induction of Rem activity (FIGS. 1M&N), as well as Rev and Rex activity. Nevertheless, LKΔHP did not abolish the increased Rem activity. Also, deletion of the hairpin from pLKO.1 still induced the Rem levels as determined by Western blotting (FIG. 10) and enhanced the GFP signal from GFPRem when visualized under a fluorescence microscope (FIG. 2A-F).

Results from the co-transfection of pLKO.1 or LKΔHP with EGFPN1, an enhanced GFP expression vector, or ER-mCherry, a RFP-expressing ER marker, suggested that induction by pLKO.1 is not limited to Rev-like proteins (FIG. 2G-K). Transfection of pLKO.1 did not affect the levels of endogenous cellular proteins as determined by Coomassie Blue staining of total transfected cell extracts. Therefore, the primary effect of pLKO.1 was on the expression or activity of newly introduced genes.

Two additional pLKO.1 deletion mutants were designed: one with a deletion from the hairpin to the pol III promoter U6 (LKΔU6) and a second with a deletion encompassing the Psi packaging signal (LKΔPsi) (FIG. 1L). Whereas LKΔU6 behaved like LKΔHP, LKΔPsi further decreased Rem protein expression levels (FIG. 1L, 1M and 1N). No difference in GFP signal intensity was detectable under a fluorescence microscope. Normalized Rem activity also was unchanged. LKΔPsi slightly decreased firefly luciferase values from a control vector. These experiments suggested that lentiviral sequences within the HIV genome, including the untranslated and packaging regions, influenced expression of co-transfected genes.

To characterize the potential effector sequences in the LKO.1 plasmid, the non-lentiviral expression vectors, E6AP-3 and HPV18E6, were employed in transfections to compare their effects with pLKO.1. Neither of these pCEP4-based shRNA expressing vectors caused super-induction of Rem activity.

Therefore, the shRNA in pLKO.1c has the greatest single effect on boosting expression of co-transfected genes. Nevertheless, combinations of structured RNAs appear to have an additive effect.

D. Effect of pLKO.1c Super-Induction Sequences Subcloned into a Vector.

Based on the results herein showing lentiviral sequences and shRNA contributions to super-induction, the lentiviral sequences and the shRNA were subcloned into another vector to determine whether the entire sequence from pLKO.1c was responsible for super-induction.

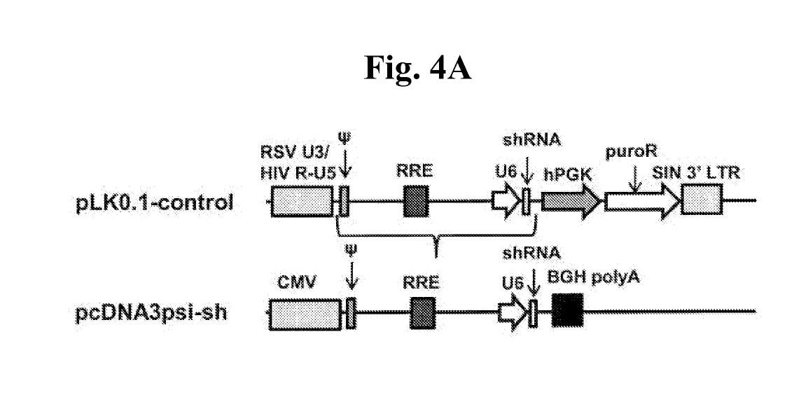

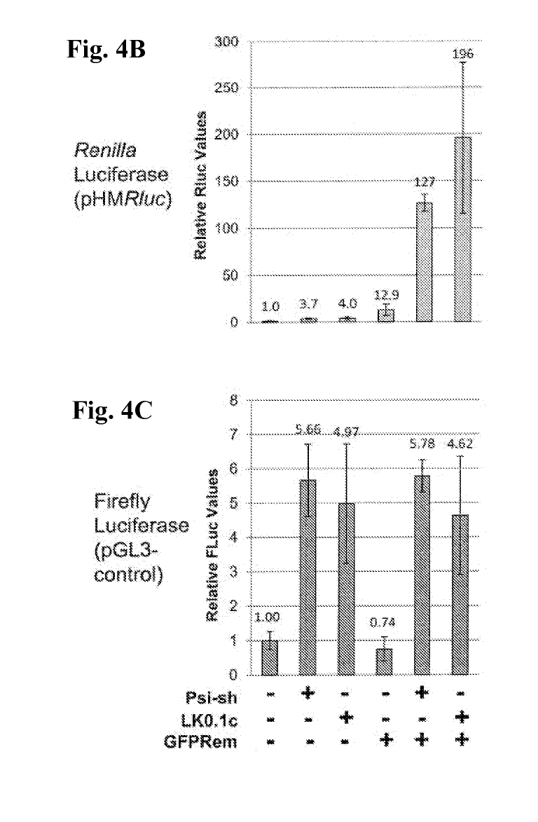

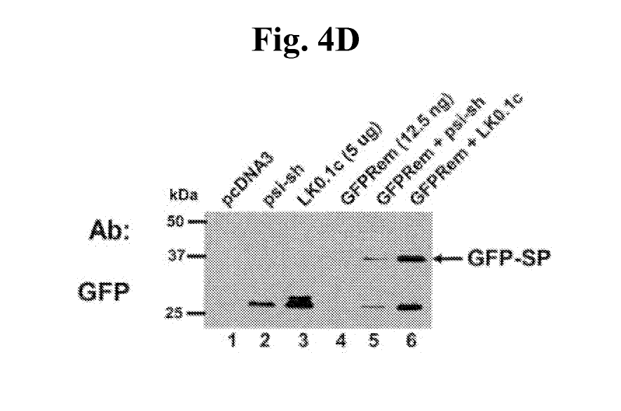

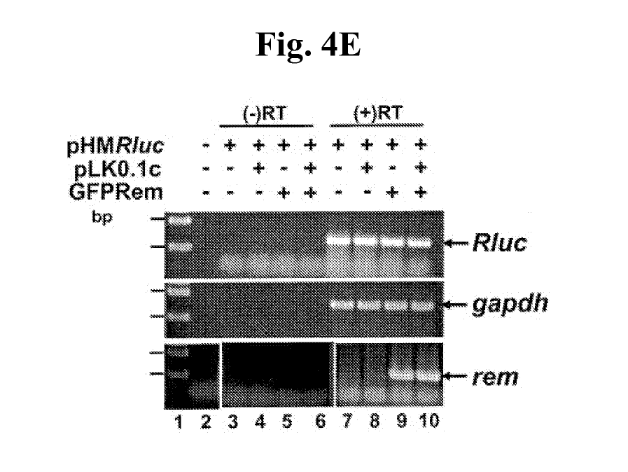

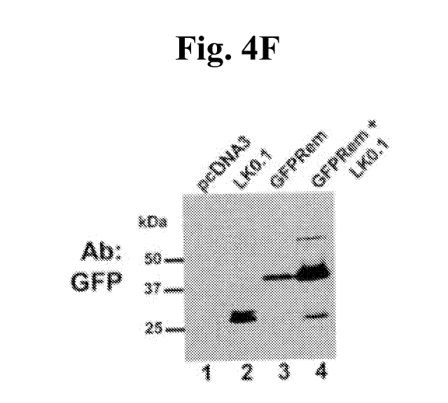

A portion of pLKO.1c containing the packaging signal ψ, the Rev-responsive element (RRE), as well as the U6 promoter upstream of the shRNA and the shRNA were subcloned into the multiple cloning site of plasmid vector, pcDNA3 (Invitrogen) (FIG. 4A). This new construct, pcDNApsi-sh, was co-transfected into 293 cells with both Rem-responsive and non-responsive vectors in the presence and absence of Rem expression.

Surprisingly, co-transfection of pcDNApsi-sh increased Renilla luciferase expression about 4-fold in the absence of Rem (FIGS. 4B&C). A 4- to 5-fold elevation of the co-transfected pGL-3 control plasmid also was observed in the presence of either pcDNApsi-sh or pLKO.1c. Addition of the Rem expression plasmid increased Renilla luciferase activity by approximately 13-fold over that observed in the absence of Rem for both plasmids. As expected, firefly luciferase expression was not responsive to Rem. Western blotting of extracts from these transfections indicated that pcDNA3psi-sh increased Rem expression, but not to same level as pLKO.1c (FIG. 4D). These experiments suggested that transfected DNA vectors that express highly structured RNAs may lead to increased expression of co-transfected genes in trans.

To determine whether pLKO.1c sequences induced changes in transcription of co-transfected genes, transfection experiments with Rem expression and a reporter vector were performed in the presence and absence of pLKO.1c. After 48 hr, cells were extracted for RNA, and RNA samples were used for semi-quantitative RT-PCR with primers specific for Rem or Renilla luciferase. The results indicated that pLKO.1c did not affect the steady state level of Rem or Rluc mRNAs (FIG. 4E) despite when protein levels increased (FIG. 4F). RT-PCRs specific for the endogenous housekeeping gene, gapdh, confirmed that uniform levels of cDNA were used for all reactions.

Together with previous results described herein, these experiments indicated that the introduction of a subset of sequences in pLKO.1c in multiple cell types leads to increased translation of genes introduced simultaneously on different vectors.

Although one embodiment of this method does not involve recloning of genes on other expression vectors, such cloning, as described above, is contemplated as part of other embodiments. In one embodiment, the super-induction retroviral sequences are removed (by endonucleases) or copied from (by PCR) from the pLKO.1 vector or other vectors containing such sequences, for use in providing super-induction vectors. In particular, the super-induction retroviral sequences are copied from the pLKO.1 vector by PCR, i.e. in one embodiment, copying the nucleic acid sequence (i.e. unit) that was removed from the pLKO.1 vector for providing a LKΔPsi mutant. In one embodiment, the super-induction retroviral sequences encompass the Psi HIV packaging signal through the U6 promoter. In one embodiment, the super-induction unit sequences encompass the Psi HIV packaging signal through the hairpin structure (shRNA). As one example described herein, the psi-sh plasmid has an inserted portion from pLKO.1c containing the packaging signal ψ, the Rev-response element (RRE), as well as the U6 promoter upstream of the shRNA and the shRNA into the multiple cloning site of the pcDNA3 plasmid vector, creating pcDNApsi-sh. Thus, in one embodiment, the super-induction retroviral sequences along with the shRNA sequences would be subcloned into an existing therapeutic vector as a cis-acting element. In one embodiment, the super-induction retroviral sequences along with the shRNA sequences would be subcloned into another plasmid or viral vector for co-transfection or introduction into a particular cell type. Thus, in other embodiments, the super-induction retroviral sequences along with the shRNA sequences may be subcloned into any vector for use as a therapeutic or vaccination vector as a retrovirus- or plasmid-based vector that increases levels of transfected genes encoding proteins that are co-introduced into mammalian cells.

E. Effect of the Transfection Process on Exogenous Gene Induction.

An additional factor that may affect Rem super-induction by pLKO.1 is the transfection process itself, which introduces some level of cellular stress. When cells are exposed to stress, stress-sensing kinases phosphorylate the cc subunit of eukaryotic translation initiation factor eIF2, leading to the inhibition of translation. This phenomenon was not observed by co-transfection of a highly structured RNA in the manner of the present invention; instead, highly structured RNA induced translation of a co-transfected gene. Western blotting with cell extracts of transfected cells, without pLKO.1, showed that transfected cells had an increased level of phosphorylated-eIF2α. Also, a dramatic increase in the cellular production of stress granules was observed in transfected cells. Surprisingly, the transfection of pLKO.1 decreased the number of cells inducing stress granules, implying that pLKO.1 relieved cellular stress or affected (lowered) granule formation to some degree. Stress granules are formed under various unfavorable conditions, resulting in transient storage of mRNAs and inhibition of translation of housekeeping proteins. The net effect is enhancement of the translation of chaperone and repair proteins to relieve stress and assist in cellular maintenance (Anderson and Kedersha. “Stressful initiations.” J. Cell. Sci. 115: 3227-3234, 2002; Anderson and Kedersha, “RNA granules.” J. Cell Biol. 172: 803-808 2006.). By relieving cellular stress levels, pLKO.1 may be able to stimulate translation of newly synthesized mRNA.

Viruses are known to influence their expression through stress granules (Lloyd, “Regulation of stress granules and P-bodies during RNA virus infection.” Wiley Interdiscip Rev RNA 4(3):317-31 (2013)). Specifically, flaviviruses and picornaviruses encode a single polyprotein precursor that are cleaved into functional proteins by virally encoded proteases. These proteases also cleave a variety of cell proteins, including G3BP, relieving the translational suppression of stress granules (Ng, et al., “Encephalomyocarditis virus disrupts stress granules, the critical platform for triggering antiviral innate immune responses.” J Virol. 87(17):9511-22 (2013)).

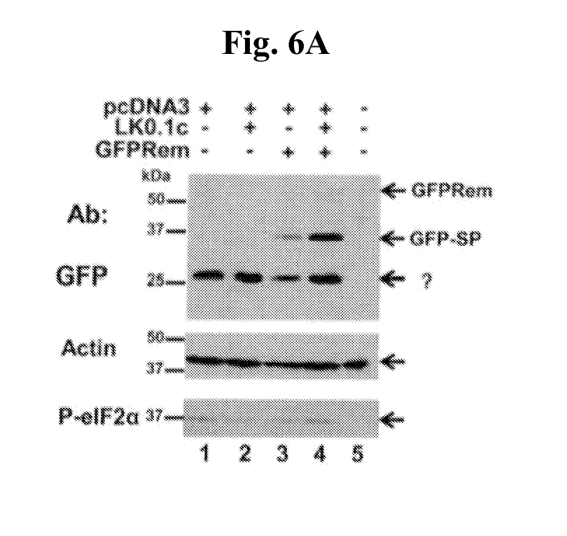

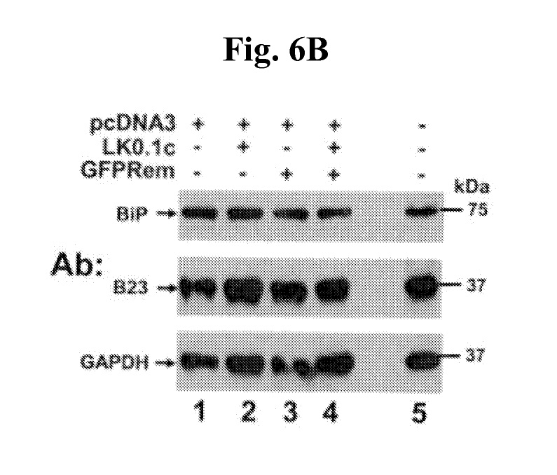

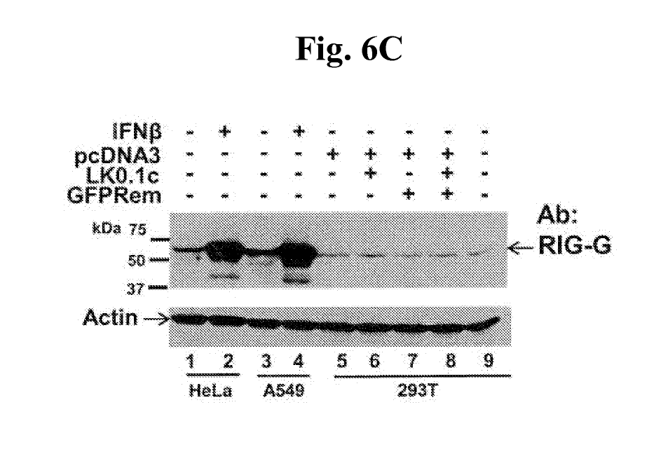

Although retroviruses also produce polyprotein precursors that are cleaved by virus-specific proteases, these proteases are believed to be sequestered in viral particles and have no known cellular targets. Analysis of cells co-transfected with pLKO.1c indicated no differences in levels or phosphorylation of G3BP. Nevertheless, unlike most plasmid-based vectors, overexpression of TIA-1, but not G3BP, stimulated Rem-mediated reporter levels. Expression of these RNA-binding proteins reduced pLKO.1c-mediated super-induction of Rem activity. These results suggest that a highly structured RNA, such as an shRNA or a retroviral sequence, can trigger a signaling pathway that prevents translational suppression of exogenous genes. This pathway can distinguish between exogenous and endogenous genes since p-actin, B23, GAPDH, and G3BP endogenous levels were unaffected by co-transfection of retroviral vectors (FIG. 6B). A cellular stress response results from a variety of conditions, including DNA damage, hypoxia, heat shock, unfolded proteins, foreign nucleic acids, nutrient deprivation, and virus infection (Lee and Ozcan, “Unfolded protein response signaling and metabolic diseases.” J Biol Chem., 289(3):1203-11. 2014, Epub 2013). Many of these stressful conditions activate a set of cellular serine/threonine kinases, including GCN2, PERK/PEK, HRI, and PKR, resulting in the phosphorylation of eIF2α (Donnelly, et al., “The eIF2α kinases: their structures and functions.” Cell Mol Life Sci., 70(19):3493-511, Epub 2013). Phosphorylated eIF2α leads to formation of a cellular protein complex that is inactive for translation initiation (Wek, et al., “Coping with stress: eIF2 kinases and translational control.” Biochem Soc Trans., 34(Pt 1):7-11. 2006). The mechanism of signaling by retroviruses to relieve stress granule formation is unknown, but has been reported to occur through virally encoded proteins (Lai, et al., “Human DDX3 interacts with the HIV-1 Tat protein to facilitate viral mRNA translation.” PLoS One 8(7):e68665 (2013)). This data on the co-transfection of pLKO.1c was inconsistent with changes to eIF2α phosphorylation (FIG. 6A). pLKO.1c transfection did not prevent arsenite-induced stress granules. Signaling through p38 also can lead to stress remediation, yet inclusion of the p38 inhibitor (SB203580) had no effect on induction of co-transfected genes by pLKO.1c. A Mnk1 inhibitor (CGP57380) also had no observable effect.

Infection of cells with RNA viruses is recognized by host RNA sensors, such as PKR, TLR-7 and TLR-8, as well as RIG-I and MDA-5 (Schlee, “Master sensors of pathogenic RNA-RIG-I like receptors.” Immunobiology, 218(11):1322-35 (2013)), leading to the synthesis of interferon and activation of NFκB. Nevertheless, an NFκB reporter was not stimulated by pLKO.1c, and increased levels of the type I interferon-stimulated gene RIG-G were not observed in the presence of this lentivirus vector (FIG. 6C).

pLKO.1, therefore, has the potential to induce several cellular reactions: an siRNA signaling pathway, the interferon response, and cellular stress. The cellular response to the structured RNA elements in pLKO O.1 leads to the super-induction of Rem activity. Recently, Bridge, et al. showed that an shRNA-expressing vector induced the IFN-inducible gene, OAS (Bridge, et al., “Induction of an interferon response by RNAi vectors in mammalian cells.” Nat. Genet. 34: 263-264 2003). However, shRNA-induced upregulation of the IFN pathway was not observed after co-transfections with pLKO.1 as detected by Western blotting for RIG-G or ISG15. The expression level of B23, a known Rem and Rev-binding protein localized to the nucleolus, showed no differences in levels by Western blotting after cell transfections with pLKO.1. The decrease of stress granules due to pLKO.1 might activate translation, leading to the increased expression levels of rem or other newly introduced genes.

In summary, the commercially available shRNA expression vector, pLKO.1, unexpectedly super-induced expression of co-transfected genes, for example, resulting in increased Rem activity and increased Rem protein levels. Experiments with a series of deletion mutants showed that the hairpin structure may be the primary factor causing super-induction of Rem activity. Nevertheless, sequences within the HIV genome also appear to contribute to increased Rem activity. pLKO.1 transfection reduced the formation of stress granules. The reduction of stress granules may release sequestered cellular mRNAs that are involved in increased Rem expression levels and the levels of co-transfected genes.

Increased expression of co-transfected genes with the lentivirus vector pLL3.7, was also observed, although the greatest increase in expression was observed with pLKO.1c expressing particular shRNAs. Removal of the shRNA from the pLKO.1c vector greatly reduced its effect on levels of co-transfected genes, indicating that shRNAs lead to further super-induction of reporter genes.

### II. Super-Induction Plasmid Vectors; Super-Induction Genes and Nucleic Acids.

The following describes exemplary super-induction vectors (plasmids) and retroviral elements of the present inventions for use in co-transfection with a gene of interest on another plasmid (vector). Super-induction vectors (plasmids) included but are not limited to a pLKO.1-puro control, a pLKO.1-4250, and a pLKO.1-4252 purchased from Sigma. Thus, in one embodiment, a super-induction vector for use is selected from the group consisting of a pLKO.1-puro control, a pLKO.1-4250, and a pLKO.1-4252.

A. shRNA Super-Induction Plasmids.

Plasmids that caused super-induction in a co-transfection system are described below. A pLKO.1-puro control (pLKO.1c) plasmid vector comprises a non-Mammalian shRNA, with no known target sequence, preinserted into the pLKO.1-puro plasmid, having SEQ ID NO:01: ccggcaacaagatgaagagcaccaactcgagttggtgctcttcatcttgttgttttt. A pLKO.1-puro-4250 plasmid (shRNA TRCN0000004250) and a pLKO.1-puro-4252 (shRNA TRCN0000004252) plasmid has hairpin shRNA TRCN0000004250 sequence SEQ ID NO:02: 5′ccgg-cctagcccttattgcattgtt-ctcgag-aacaatgcaataagggctagg-ttftt3′, and shRNA TRCN0000004252 SEQ ID NO:03: 5′CCGG-gatggatgaattgcagagtt-ctcgag-aacaactgcaattcatccatc-ttat3′, respectively.

Although an shRNA target sequence for the pLKO.1-control (pLKO.1c) has not been identified, pLK-4250, and pLK-4252 plasmids have DNA targets i.e. ATPase p97 shRNA AAA A: CCTAGCCCTTATTGCATTGTT, SEQ ID NO:04, (Human VCP) and GATGGATGAATTGCAGTTGTT [SEQ ID NO:05] (Human VCP), respectively. During the development of the present inventions, unlike transfection of pLK-4252, transfection of pLK-4250 decreased p97/VCP protein levels. Valosin-containing protein (VCP) or p97, refers to a type II member of AAA+-ATPase familyATPases having multiple cellular activities, including ubiquitin segregase activity. Thus, in one embodiment, a super-induction vector comprises a highly structured RNA, wherein the highly structured DNA has a sequence selected from the group consisting of SEQ ID NO:01, SEQ ID NO:02 and SEQ ID NO:03. This statement is not meant to limit the shRNA sequence, since any shRNA sequence may be capable of a super-induction effect and may be useful in a super-induction plasmid of the present inventions. Thus in one embodiment, a shRNA preferably does not have a known target gene.

As one example, a shRNA was cloned into Agel and EcoRI sites of the multicloning site of the following LKO.1, (pLKO hPGK-puro (direct) 7052 bp (no shRNA insert; empty vector) SEQ ID NO:06 for providing a super-induction vector of the present inventions:

TTGGGGTTGCGCCTTTTCCAAGGCAGCCCTGGGTTTGCGCAGGGACGCGG
CTGCTCTGGGCGTGGTTCCGGGAAACGCAGCGGCGCCGACCCTGGGTCTC
GCACATTCTTCACGTCCGTTCGCAGCGTCACCCGGATCTTCGCCGCTACC
CTTGTGGGCCCCCCGGCGACGCTTCCTGCTCCGCCCCTAAGTCGGGAAGG
TTCCTTGCGGTTCGCGGCGTGCCGGACGTGACAAACGGAAGCCGCACGTC
TCACTAGTACCCTCGCAGACGGACAGCGCCAGGGAGCAATGGCAGCGCGC
CGACCGCGATGGGCTGTGGCCAATAGCGGCTGCTCAGCAGGGCGCGCCGA
GAGCAGCGGCCGGGAAGGGGCGGTGCGGGAGGCGGGGTGTGGGGCGGTAG
TGTGGGCCCTGTTCCTGCCCGCGCGGTGTTCCGCATTCTGCAAGCCTCCG
GAGCGCACGTCGGCAGTCGGCTCCCTCGTTGACCGAATCACCGACCTCTC
TCCCCAGGGGGATCCACCGGAGCTTACCATGACCGAGTACAAGCCCACGG
TGCGCCTCGCCACCCGCGACGACGTCCCCAGGGCCGTACGCACCCTCGCC
GCCGCGTTCGCCGACTACCCCGCCACGCGCCACACCGTCGATCCGGACCG
CCACATCGAGCGGGTCACCGAGCTGCAAGAACTCTTCCTCACGCGCGTCG
GGCTCGACATCGGCAAGGTGTGGGTCGCGGACGACGGCGCCGCGGTGGCG
GTCTGGACCACGCCGGAGAGCGTCGAAGCGGGGGCGGTGTTCGCCGAGAT
CGGCCCGCGCATGGCCGAGTTGAGCGGTTCCCGGCTGGCCGCGCAGCAAC
AGATGGAAGGCCTCCTGGCGCCGCACCGGCCCAAGGAGCCCGCGTGGTTC
CTGGCCACCGTCGGCGTCTCGCCCGACCACCAGGGCAAGGGTCTGGGCAG
CGCCGTCGTGCTCCCCGGAGTGGAGGCGGCCGAGCGCGCCGGGGTGCCCG
CCTTCCTGGAGACCTCCGCGCCCCGCAACCTCCCCTTCTACGAGCGGCTC
GGCTTCACCGTCACCGCCGACGTCGAGGTGCCCGAAGGACCGCGCACCTG
GTGCATGACCCGCAAGCCCGGTGCCTGACGCCCGCCCCACGACCCGCAGC
GCCCGACCGAAAGGAGCGCACGACCCCATGCATCGGTACCTTTAAGACCA
ATGACTTACAAGGCAGCTGTAGATCTTAGCCACTTTTTAAAAGAAAAGGG
GGGACTGGAAGGGCTAATTCACTCCCAACGAAGACAAGATCTGCTTTTTG
CTTGTACTGGGTCTCTCTGGTTAGACCAGATCTGAGCCTGGGAGCTCTCT
GGCTAACTAGGGAACCCACTGCTTAAGCCTCAATAAAGCTTGCCTTGAGT
GCTTCAAGTAGTGTGTGCCCGTCTGTTGTGTGACTCTGGTAACTAGAGAT
CCCTCAGACCCTTTTAGTCAGTGTGGAAAATCTCTAGCAGTAGTAGTTCA
TGTCATCTTATTATTCAGTATTTATAACTTGCAAAGAAATGAATATCAGA
GAGTGAGAGGAACTTGTTTATTGCAGCTTATAATGGTTACAAATAAAGCA
ATAGCATCACAAATTTCACAAATAAAGCATTTTTTTCACTGCATTCTAGT
TGTGGTTTGTCCAAACTCATCAATGTATCTTATCATGTCTGGCTCTAGCT
ATCCCGCCCCTAACTCCGCCCATCCCGCCCCTAACTCCGCCCAGTTCCGC
CCATTCTCCGCCCCATGGCTGACTAATTTTTTTTATTTATGCAGAGGCCG
AGGCCGCCTCGGCCTCTGAGCTATTCCAGAAGTAGTGAGGAGGCTTTTTT
GGAGGCCTAGGGACGTACCCAATTCGCCCTATAGTGAGTCGTATTACGCG
CGCTCACTGGCCGTCGTTTTACAACGTCGTGACTGGGAAAACCCTGGCGT
TACCCAACTTAATCGCCTTGCAGCACATCCCCCTTTCGCCAGCTGGCGTA
ATAGCGAAGAGGCCCGCACCGATCGCCCTTCCCAACAGTTGCGCAGCCTG
AATGGCGAATGGGACGCGCCCTGTAGCGGCGCATTAAGCGCGGCGGGTGT
GGTGGTTACGCGCAGCGTGACCGCTACACTTGCCAGCGCCCTAGCGCCCG
CTCCTTTCGCTTTCTTCCCTTCCTTTCTCGCCACGTTCGCCGGCTTTCCC
CGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTG
GGCCATCGCCCTGATAGACGGTTTTTCGCCCTTTGACGTTGGAGTCCACG
TTCTTTAATAGTGGACTCTTGTTCCAAACTGGAACAACACTCAACCCTAT
CTCGGTCTATTCTTTTGATTTATAAGGGATTTTGCCGATTTCGGCCTATT
GGTTAAAAAATGAGCTGATTTAACAAAAATTTAACGCGAATTTTAACAAA
ATATTAACGCTTACAATTTAGGTGGCACTTTTCGGGGAAATGTGCGCGGA
ACCCCTATTTGTTTATTTTTCTAAATACATTCAAATATGTATCCGCTCAT
GAGACAATAACCCTGATAAATGCTTCAATAATATTGAAAAAGGAAGAGTA
TGAGTATTCAACATTTCCGTGTCGCCCTTATTCCCTTTTTTGCGGCATTT
TGCCTTCCTGTTTTTGCTCACCCAGAAACGCTGGTGAAAGTAAAAGATGC
TGAAGATCAGTTGGGTGCACGAGTGGGTTACATCGAACTGGATCTCAACA
GCGGTAAGATCCTTGAGAGTTTTCGCCCCGAAGAACGTTTTCCAATGATG
AGCACTTTTAAAGTTCTGCTATGTGGCGCGGTATTATCCCGTATTGACGC
CGGGCAAGAGCAACTCGGTCGCCGCATACACTATTCTCAGAATGACTTGG
TTGAGTACTCACCAGTCACAGAAAAGCATCTTACGGATGGCATGACAGTA
AGAGAATTATGCAGTGCTGCCATAACCATGAGTGATAACACTGCGGCCAA
CTTACTTCTGACAACGATCGGAGGACCGAAGGAGCTAACCGCTTTTTTGC
ACAACATGGGGGATCATGTAACTCGCCTTGATCGTTGGGAACCGGAGCTG
AATGAAGCCATACCAAACGACGAGCGTGACACCACGATGCCTGTAGCAAT
GGCAACAACGTTGCGCAAACTATTAACTGGCGAACTACTTACTCTAGCTT
CCCGGCAACAATTAATAGACTGGATGGAGGCGGATAAAGTTGCAGGACCA
CTTCTGCGCTCGGCCCTTCCGGCTGGCTGGTTTATTGCTGATAAATCTGG
AGCCGGTGAGCGTGGGTCTCGCGGTATCATTGCAGCACTGGGGCCAGATG
GTAAGCCCTCCCGTATCGTAGTTATCTACACGACGGGGAGTCAGGCAACT
ATGGATGAACGAAATAGACAGATCGCTGAGATAGGTGCCTCACTGATTAA
GCATTGGTAACTGTCAGACCAAGTTTACTCATATATACTTTAGATTGATT
TAAAACTTCATTTTTAATTTAAAAGGATCTAGGTGAAGATCCTTTTTGAT
AATCTCATGACCAAAATCCCTTAACGTGAGTTTTCGTTCCACTGAGCGTC
AGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTT
CAGCAGAGCGCAGATACCAAATACTGTTCTTCTAGTGTAGCCGTAGTTAG
GCCACCACTTCAAGAACTCTGTAGCACCGCCTACATACCTCGCTCTGCTA
ATCCTGTTACCAGTGGCTGCTGCCAGTGGCGATAAGTCGTGTCTTACCGG
GTTGGACTCAAGACGATAGTTACCGGATAAGGCGCAGCGGTCGGGCTGAA
CGGGGGGTTCGTGCACACAGCCCAGCTTGGAGCGAACGACCTACACCGAA
CTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGC
GCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCC
TGGCCTTTTGCTGGCCTTTTGCTCACATGTTCTTTCCTGCGTTATCCCCT
GATTCTGTGGATAACCGTATTACCGCCTTTGAGTGAGCTGATACCGCTCG
CCGCAGCCGAACGACCGAGCGCAGCGAGTCAGTGAGCGAGGAAGCGGAAG
AGCGCCCAATACGCAAACCGCCTCTCCCCGCGCGTTGGCCGATTCATTAA
TGCAGCTGGCACGACAGGTTTCCCGACTGGAAAGCGGGCAGTGAGCGCAA
CGCAATTAATGTGAGTTAGCTCACTCATTAGGCACCCCAGGCTTTACACT
TTATGCTTCCGGCTCGTATGTTGTGTGGAATTGTGAGCGGATAACAATTT
CACACAGGAAACAGCTATGACCATGATTACGCCAAGCGCGCAATTAACCC
TCACTAAAGGGAACAAAAGCTGGAGCTGCAAGCTTAATGTAGTCTTATGC
AATACTCTTGTAGTCTTGCAACATGGTAACGATGAGTTAGCAACATGCCT
TACAAGGAGAGAAAAAGCACCGTGCATGCCGATTGGTGGAAGTAAGGTGG
TACGATCGTGCCTTATTAGGAAGGCAACAGACGGGTCTGACATGGATTGG
ACGAACCACTGAATTGCCGCATTGCAGAGATATTGTATTTAAGTGCCTAG
CTCGATACATAAACGGGTCTCTCTGGTTAGACCAGATCTGAGCCTGGGAG
CTCTCTGGCTAACTAGGGAACCCACTGCTTAAGCCTCAATAAAGCTTGCC
TTGAGTGCTTCAAGTAGTGTGTGCCCGTCTGTTGTGTGACTCTGGTAACT
AGAGATCCCTCAGACCCTTTTAGTCAGTGTGGAAAATCTCTAGCAGTGGC
GCCCGAACAGGGACTTGAAAGCGAAAGGGAAACCAGAGGAGCTCTCTCGA
CGCAGGACTCGGCTTGCTGAAGCGCGCACGGCAAGAGGCGAGGGGCGGCG
ACTGGTGAGTACGCCAAAAATTTTGACTAGCGGAGGCTAGAAGGAGAGAG
ATGGGTGCGAGAGCGTCAGTATTAAGCGGGGGAGAATTAGATCGCGATGG
GAAAAAATTCGGTTAAGGCCAGGGGGAAAGAAAAAATATAAATTAAAACA
TATAGTATGGGCAAGCAGGGAGCTAGAACGATTCGCAGTTAATCCTGGCC
TGTTAGAAACATCACGAAGGCTGTAGACAAATACTGGGACAGCTACAACC
ATCCCTTCAGACAGGATCAGAAGAACTTACATCATTATATAATACAGTAG
CAACCCTCTATTGTGTGCATCAAAGGATAGAGATAAAAGACACCAAGGAA
GCTTTAGACAAGATAGAGGAAGAGCAAAACAAAAGTAAGACCACCGCACA
GCAAGCGGCCGCTGATCTTCAGACCTGGAGGAGGAGATATGAGGGACAAT
TGGAGAAGTGAATTATATAAATATAAAGTAGTAAAAATTGAACCATTAGG
AGTAGCACCCACCAAGGCAAAGAGAAGAGTGGTGCAGAGAGAAAAAAGAG
CAGTGGGAATAGGAGCTTTGTTCCTTGGGTTCTTGGGAGCAGCAGGAAGC
ACTATGGGCGCAGCGTCAATGACGCTGACGGTACAGGCCAGACAATTATT
GTCTGGTATAGTGCAGCAGCAGAACAATTTGCTGAGGGCTATTGAGGCGC
AACAGCATCTGTTGCAACTCACAGTCTGGGGCATCAAGCAGCTCCAGGCA
AGAATCCTGGCTGTGGAAAGATACCTAAAGGATCAACAGCTCCTGGGGAT
TTGGGGTTGCTCTGGAAAACTCATTTGCACCACTGCTGTGCCTTGGAATG
CTAGTTGGAGTAATAAATCTCTGGAACAGATTTGGAATCACACGACCTGG
ATGGAGTGGGACAGAGAAATTAACAATTACACAAGCTTAATACACTCCTT
AATTGAAGAATCGCAAAACCAGCAAGAAAAGAATGAACAAGAATTATTGG
AATTAGATAAATGGGCAAGTTTGTGGAATTGGTTTAACATAACAAATTGG
CTGTGGTATATAAAAATTATTCATAATGATAGTAGGAGGCTTGGTAGGTT
TAAGAATAGTTTTTGCTGTACTTTCTATAGTGAATAGAGTTAGGCAGGGA
TATTCACCATTATCGTTTCAGACCCACCTCCCAACCCCGAGGGGACCCGA
CAGGCCCGAAGGAATAGAAGAAGAAGGTGGAGAGAGAGACAGAGACAGAT
CCATTCGATTAGTGAACGGATCTCGACGGTATCGATCACGAGACTAGCCT
CGAGCGGCCGCCCCCTTCACCGAGGGCCTATTTCCCATGATTCCTTCATA
TTTGCATATACGATACAAGGCTGTTAGAGAGATAATTGGAATTAATTTGA
CTGTAAACACAAAGATATTAGTACAAAATACGTGACGTAGAAAGTAATAA
TTTCTTGGGTAGTTTGCAGTTTTAAAATTATGTTTTAAAATGGACTATCA
TATGCTTACCGTAACTTGAAAGTATTTCGATTTCTTGGCTTTATATATCT
TGTGGAAAGGACGAAACACCGGTCCGCAGGTATGCACGCGTGAATTCTCG
ACCTCGAGACAAATGGCAGTATTCATCCACAATTTTAAAAGAAAAGGGGG
GATTGGGGGGTACAGTGCAGGGGAAAGAATAGTAGACATAATAGCAACAG
ACATACAAACTAAAGAATTACAAAAACAAATTACAAAAATTCAAAATTTT
CGGGTTTATTACAGGGACAGCAGAGATCCACTTTGGCCGCGGCTCGAGGG
GG.

B. Super-Induction Lentiviral Nucleic Acid Unit.

The pLKO.1-puro vector (i.e. backbone, SEQ ID NO:06) has an additional super-induction nucleic acid unit comprising a stretch of DNA derived from a lentiviral HIV. This super-induction nucleic acid unit comprises a human U6 promoter (a pol III promoter) for driving expression of the shRNA hairpin within a mammalian cell, a retroviral packaging element, packaging signal iv, and a Rev-responsive element (RRE), along with intervening sequences. In general, the super-induction lentiviral unit comprises an empty vector, i.e. a vector with no gene of interest for expression (e.g. pcDNA3). See also, Stewart, et al., “Lentivirus-delivered stable gene silencing by RNAi in primary cells.” RNA 2003 April; 9(4):493-501, herein incorporated by reference in its entirety, for use in providing a lentiviral nucleic acid unit super-induction sequences of the present inventions.

C. Other Plasmids Comprising a shRNA.

As another example, a pSTK129-shLuc-GFP plasmid, comprising a shRNA directed against luciferase, may find use as a super-induction shRNA as an shRNA adenovirus (Ad) super-induction vector, Machitani, et al., “Adenovirus Vector-Derived VA-RNA-Mediated Innate Immune Responses.” Pharmaceutics, 3(3), 338-353 (2011). Thus in another embodiment, a super-induction shRNA targets a gene not typically expressed in host mammalian cells.

### III. Structural RNA Encoding Sequences as Super-Induction Nucleic Acid Sequences.

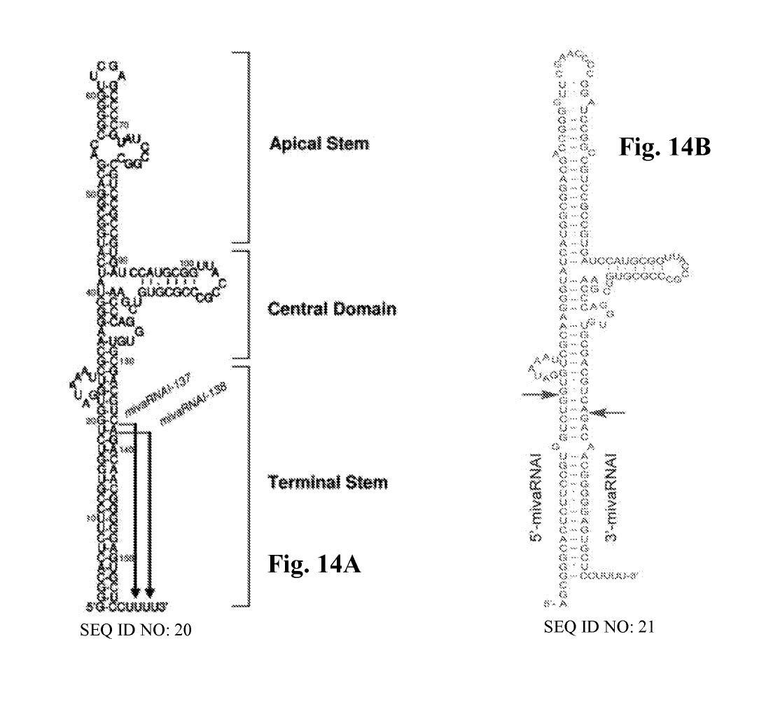

The present invention is not intended to be limited to a specific highly structured RNA so long as the sequences encoding it contribute to a super-induction response. A variety of highly structured RNA molecules are contemplated for use as super-induction sequences, including but not limited to shRNA, virus-associated RNA (VA-RNA) (Mathews and Shenk, 1991), such as in adenovirus (Ad) having at least one VA-RNA, and often two, i.e. VA-RNA-I and VA-RNA-II, adenoviral constructs retaining one or more of these sequences, in addition to other retroviral highly structured RNAs, such as the packaging sequence ¶, the transactivation-response element (TAR), internal ribosomal entry site (IRES), and highly structured 5′ and 3′ untranslated regions, and the like. As examples of a structural RNA, VA-RNA-I is shown in FIG. 14A. VA-RNA-I folds into a secondary structure comprising an apical stem, a central domain, and a terminal stem, with a portion processed into mivaRNAI-137 and mivaRNAI-138. Adenoviruses have numerous subtypes (A-F), and numerous isolates showing variations in genomic sequences, see another example in FIG. 14B.

As some examples of an Ad genome, human adenovirus type 2 (Ad2) ACCESSION AC_{—}000007; NCBI Reference Sequence: AC_{—}000007.1. and the closely related serotype Ad5 A are shown as VA-RNA I (VAI-RNA) and II (VAII-RNA) and are about 160 nucleotide-long non-coding RNAs encoded in the Ad genome. VA RNAs function to inhibit interferon-induced PKR activation (Launer-Felty and Cole, “Domain interactions in adenovirus VAI RNA mediate high-affinity PKR binding.” J Mol Biol. 426(6):1285-95 (2014)). VA-RNA-I is processed into two major species of viral miRNAs or mivaRNAs (i.e. mivaRNAI-137 (A) and 138 (G)). VA-RNA-I is processed in the cell to create 22 nucleotide long RNAs that can act as small interfering RNA ((siRNA); short interfering RNA; silencing RNA) or microRNA (miRNA). Sano, et al., “Sequence-specific interference by small RNAs derived from adenovirus VAI RNA”. FEBS Lett. 580(6):1553-1564 (2006).

As one example, Adenovirus type 2 VA-RNA I is encoded by [SEQ ID NO:08]:

gggcactcttccgtggtctggtggataaattcgcaagggtatcatggcgg
acgaccggggttcgaaccccggatccggccgtccgccgtgatccatgcgg
ttaccgcccgcgtgtegaacccaggtgtgcgacgtcagacaacgggggag
cgctcctttt.

As one example using Human adenovirus A, VA-RNA I is encoded by [SEQ ID NO:09] bp10620-10779:

a
gtcgcgtggc atttgtgcct cgtcagaaca tgtttcgcga
caacagcggg gaggaagctg aggaaatgcg agactgcagg
tttagggccg gtcgcgagct gcgccgcgga tttaatcgcg
agcgactgct gcgtgaggag gactttgagc cagatgaaca
ttcggggatt agttctgca.

10621 10681 10741

As one example, Adenovirus type 2 VA-RNA II is encoded by [SEQ ID NO:10]:

ggctcgctccctgtagccggagggttattttccaagggttgagtcgcagg
acccccggttcgagtctcgggccggccggactgcggcgaacgggggatgc
ctccccgtcatgcaagaccccgcttgcaaattcctccggaaacagggacg
agcccctttttt.

As one example using Human adenovirus A, VA-RNA II is [SEQ ID NO:11] by 10876-11038:

gcttt aataaccatg tgcgcacact aatagcgcga gaagaagtag
10921 ccattggttt aatgcatctt tgggactttg tagaagctta
tgtacataat ccagcaagta
10981 aacccctaac tgcccagctg ttcttaatag ttcaacatag
tagagacaat gaaacttt.

In some embodiments, structural RNA is contemplated for use as super-induction elements. Thus in one embodiment, a structural RNA is included as a trans-acting super-induction element in a vector co-transfected with an expression vector.

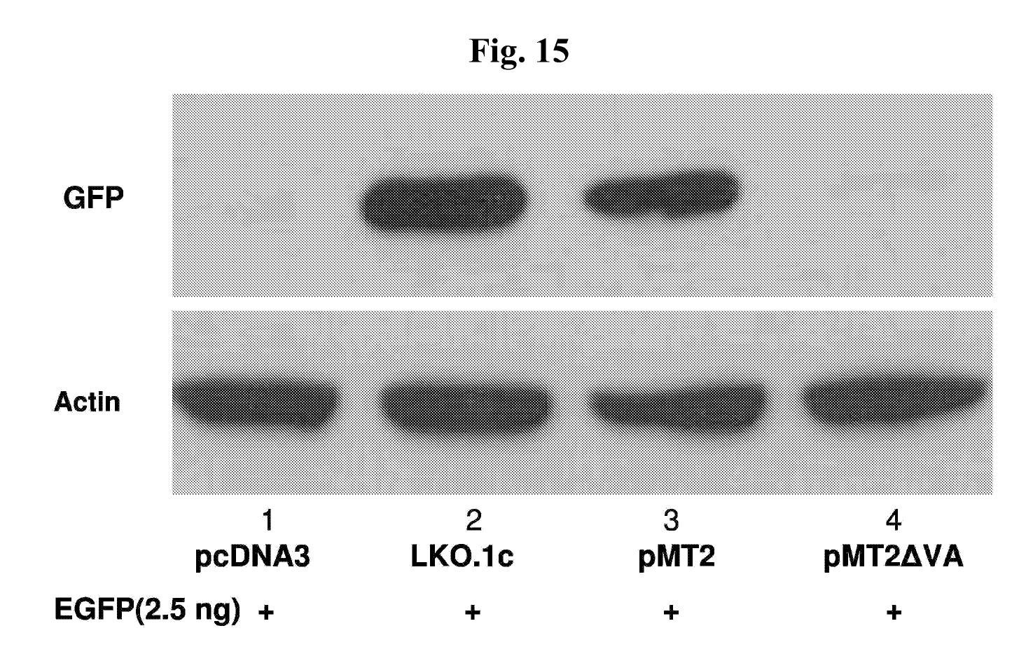

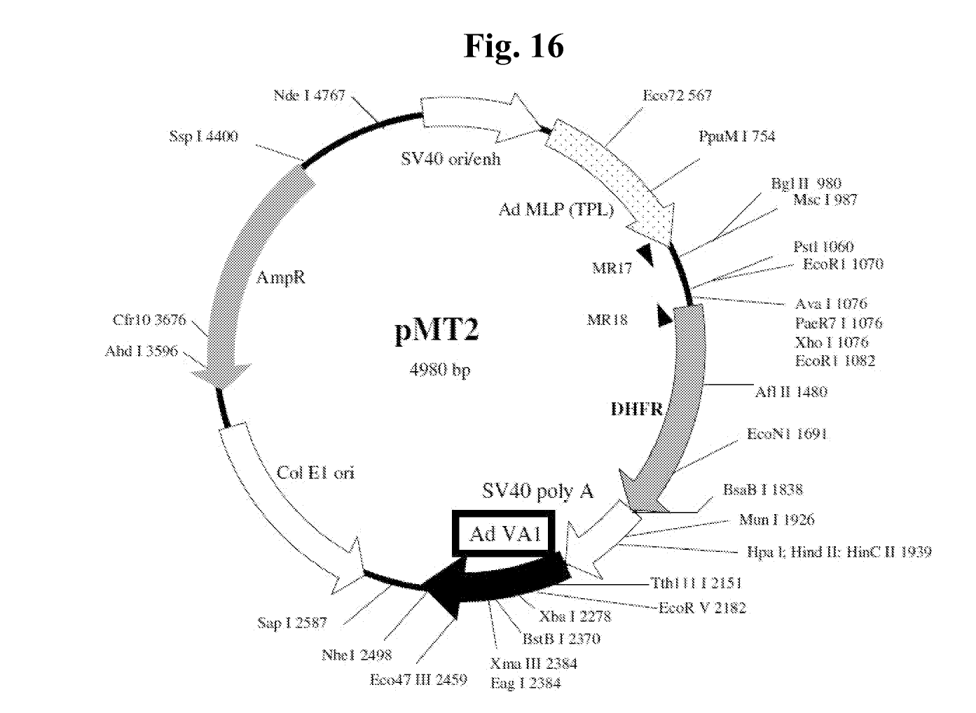

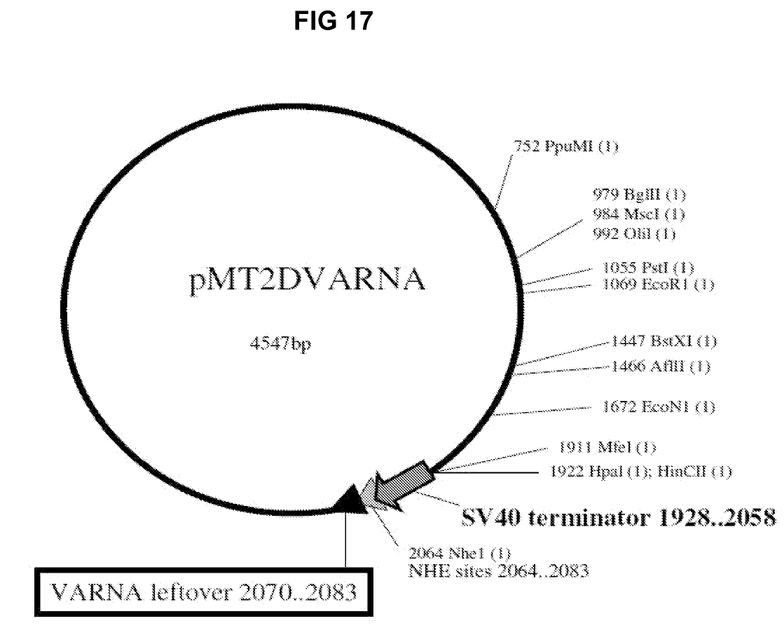

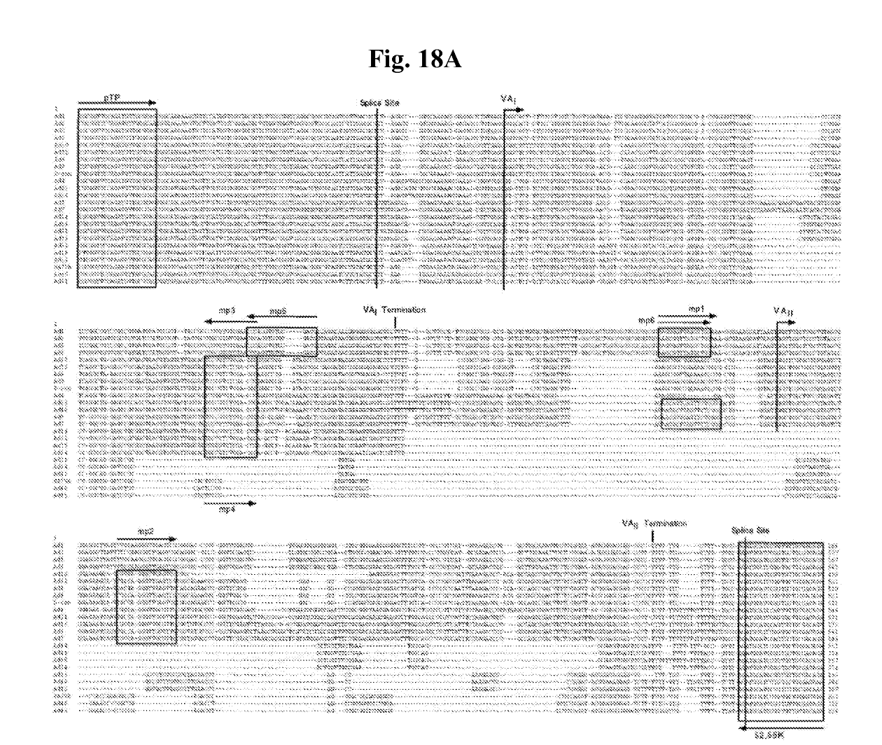

In fact, a pMT2 construct (based upon pBR322) comprising VA-RNA I, see below and in FIG. 16, showed super-induction of GFP as a trans-acting element on EGFP reporter plasmids (2.5 ng) at levels comparable to GFP induction using pLKO 1.c. See, FIG. 15. For comparison, the pMT2deltaVA plasmid (where the majority of VA-RNA I sequence was removed, see, FIG. 17) showed no induction of GFP as did the negative control pcDNA3 plasmid.

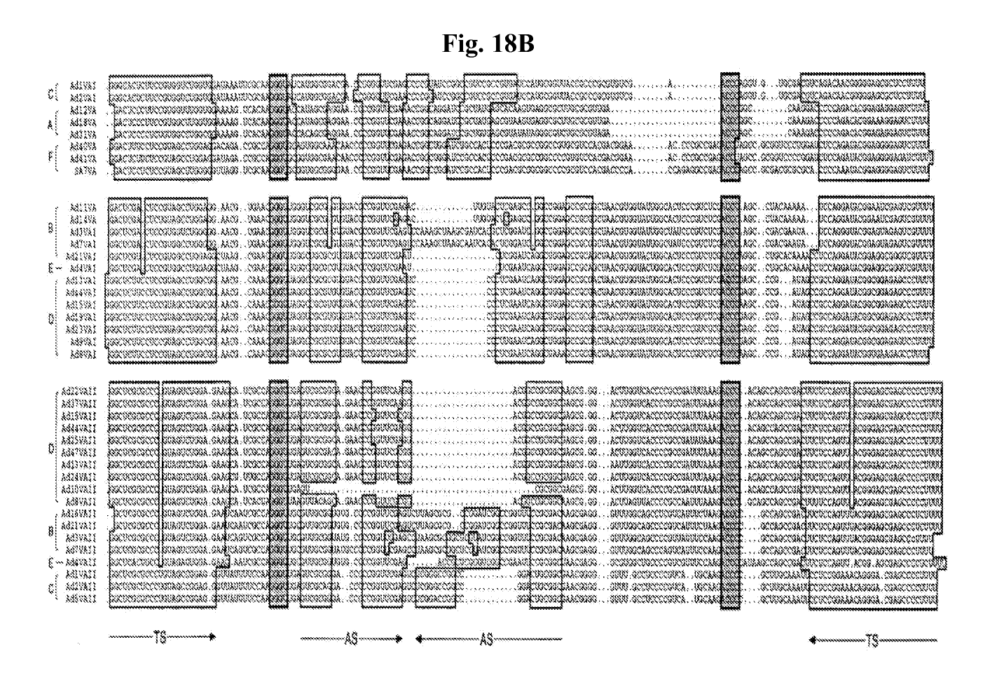

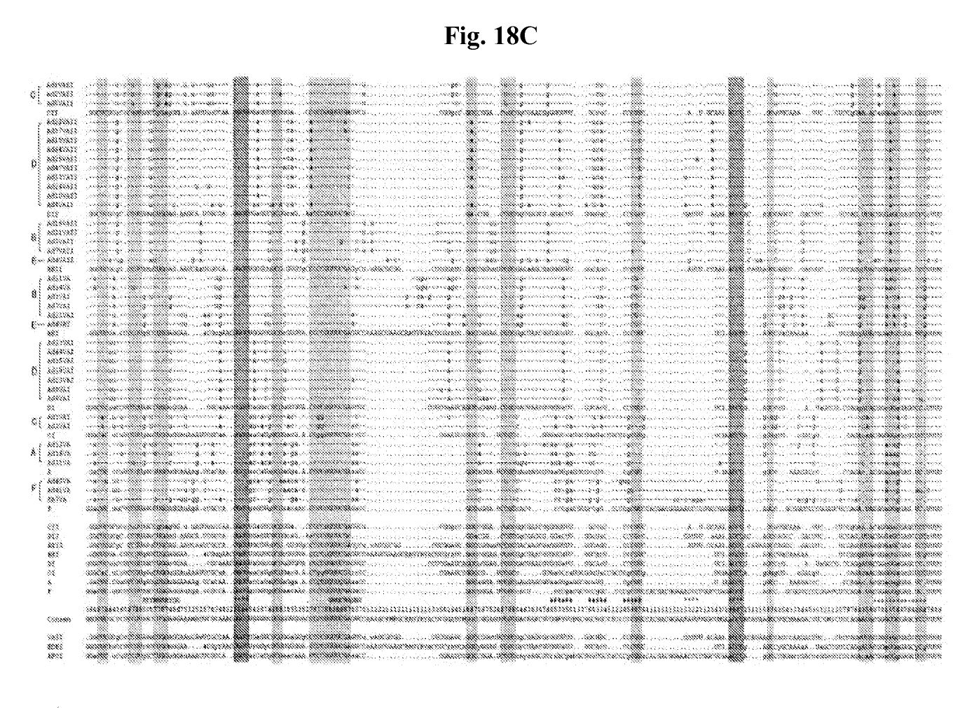

Thus, VA-RNA, including sequence variations thereof, that may be useful as structured RNA super-induction sequences of the present inventions. See, for examples of other VA-RNA sequences for use as super-induction sequences, FIG. 18A-C (FIGS. 2, 3, and 5, respectfully, in Ma and Mathews, “Structure, Function, and Evolution of Adenovirus-Associated RNA: a Phylogenetic Approach.” Journal Of Virology, 996, p. 5083-5099, (1996), all of which is herein incorporated by reference. In one embodiment, a VA-RNA may have a specific cellular or viral target. In a preferred embodiment, a VA-RNA preferably does not have a known target gene.

One example for use of VA-RNAs in a vector, either as a cis or trans acting element, is provided herein using human adenovirus type 5 (ad5), as described in Kamel, et al., “The adenovirus VA RNA-derived miRNAs are not essential for lytic virus growth in tissue culture cells.” Nucl. Acids Res. (2013). Subcloning of VA RNA I using a BstXI fragment (base pairs: 10035-14289) spanning the VA RNA region of Ad5 can be used for ligation into a super-induction plasmid (or other vector) or for ligating into an expression plasmid (or other expression vector). As one example, subcloning a VAI and/or VAII-RNA DNA sequence into a lentiviral vector (one example, WO 2005056057, herein incorporated by reference in its entirety) for use as a super-induction vector of the present inventions. In another example, a VAI and/or VAII-RNA DNA sequence is inserted into an expression vector for super-induction of a gene of interest. Thus in another embodiment, a structural RNA is included as a cis-acting element for an expression vector.

### IV. Contemplated Uses for Super-Induction Nucleic Acid Sequences and Plasmids of the Present Inventions.

The inventors contemplate using the super-induction nucleic acid sequences and plasmids of the present inventions for enhancing gene therapy and related genetic therapies relying upon transfected expression constructs.

A. Transient Production of High Titers of Retroviral Vectors for Use in Methods and Systems of the Present Inventions.

Generation of high-titer retrovirus by transient production is less laborious than production of stable retroviral producer cell lines. Transient production also allowed the production of high-titer retroviral supernatants from cDNAs that were not achieved by stable producer cell lines. Transient transfection increased the versatility of retrovirus-mediated gene transfer to include the rapid testing of different constructs, viral pseudotyping, and construction of retroviral cDNA libraries.

Systems based on human 293T cells, an adenovirus-transformed human embryonic kidney cell line, produced the highest retroviral titers for other systems and thus are the most widely used. Therefore, the inventors contemplate the use of human 293 cells for producing super-induction vectors of the present inventions. In one embodiment, the present invention contemplates co-transfecting with a vector comprising sequences corresponding to highly structured RNA (said vector preferably expressing a highly structured RNA) so as to optimize retroviral production from 293-based systems. In one embodiment, higher titers will be achieved by using 293 cell based systems.

B. Gene therapy.

Gene therapy in particular is a technically difficult procedure with great potential for treating chronic diseases, such as cancer, immunodeficiencies, and neurological degeneration. With the use of super-induction sequences described herein, the inventors contemplate therapeutic protein expression in host cells with fewer side effects. Thus, in one embodiment, a therapeutic expression vector for gene therapy is co-transfected with a super-induction structural RNA sequence of the present inventions.

C. Use of Super-Induction Sequences and Vectors for Increasing Effectiveness of DNA Vaccines.

Vaccination is a method for stimulating the immune system of a host subject with an infectious agent, or components of an infectious agent, or an antigen derived from an infectious agent. This agent is modified in such a manner that no harm or disease is caused, so that when the host is confronted with the infectious agent, the immune system can adequately neutralize it before it causes disease. DNA vaccines comprise DNA plasmids expressing genes for producing one or more antigens associated with a target pathogen (e.g. a specific type of bacteria, virus or cancer) and serve as an epigenetic template for the high-efficiency translation of its antigen inside of cells. Therefore, in general, a DNA vaccine plasmid comprises a bacterial regulatory region that provides selection and propagation in bacteria for producing large amounts of the DNA vaccine, and a eukaryotic regulatory region that directs expression of said gene of interest in a host cell.

In one embodiment, said first vector comprises a gene of interest cloned into a pVAX1 vector. pVAX1© is a 3.0 kb plasmid vector designed for use in the development of DNA vaccines (commercially available from LifeTech). The vector was constructed to be consistent with the (FDA) document, “Points to Consider on Plasmid DNA Vaccines for Preventive Infectious Disease Indications” published December 1996. Features of the vector allow high-copy number replication in E. coli and high-level transient expression of the protein of interest in most mammalian cells. The vector comprises the following elements: Human cytomegalovirus immediate-early (CMV) promoter for high-level expression in a wide range of mammalian cells; Bovine growth hormone (BGH) polyadenylation signal for efficient transcription termination and polyadenylation of mRNA; and a kanamycin-resistance gene for selection in E. coli. The control plasmid, pVAX1©/lacZ, is included for use as a positive control for transfection and expression in the cell line of choice.

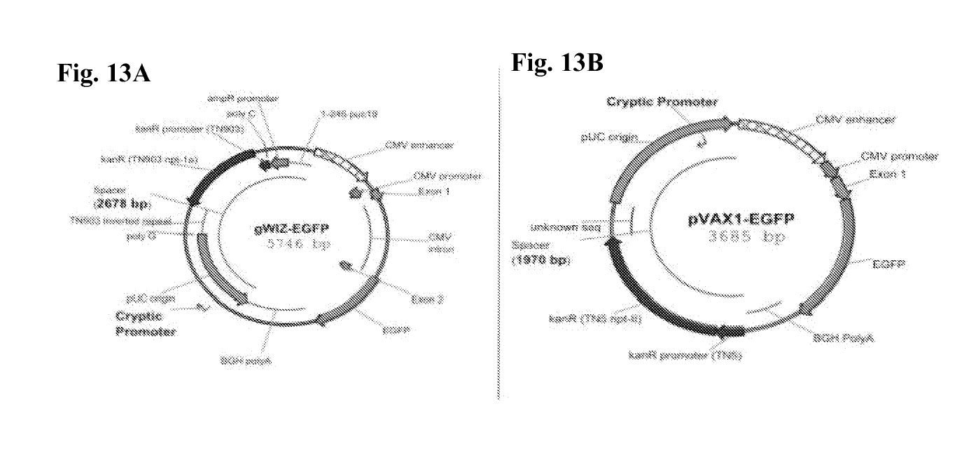

As noted above, the present inventions include increasing effectiveness of existing vaccines using highly structured RNA-expressed in cis or in trans. For example, there are at least four animal health DNA vaccine products that are licensed showing productive protection. The invention contemplates enhancing these vaccine products (along with others). For example, the present invention can enhance the utility of DNA vaccination in large animals including horses and pigs, Williams, “Vector Design for Improved DNA Vaccine Efficacy, Safety and Production.” Vaccines 1:225-249 2013. Exemplary schematics of vaccination plasmids that may find use in the present inventions are shown in FIG. 13A and FIG. 13. These licensed products include preventative vaccines for West Nile virus in horses and infectious haematopoietic necrosis virus in fish, a therapeutic cancer vaccine for dogs, see, for examples, Liu, “DNA vaccines: An historical perspective and view to the future.” Immunol. Rev. 2011, 239:62-84) and a growth hormone gene therapy to increase litter survival in breeding pig sows. DNA vaccination with influenza hemagglutinin antigen has demonstrated utility to induce broadly cross neutralizing antibodies (Wang, et al., “Heterologous HA DNA vaccine prime-inactivated influenza vaccine boost is more effective than using DNA or inactivated vaccine alone in eliciting antibody responses against H1 or H3 serotype influenza viruses.” Vaccine 2008, 26:3626-3633; Wei, et al., “Induction of broadly neutralizing H1N1 influenza antibodies by vaccination. Science 2010, 329, 1060-1064; Ledgerwood, et al., “DNA priming and influenza vaccine immunogenicity: Two phase 1 open label randomised clinical trials.” Lancet Infect. Dis. 2011, 11:916-924).

However, efficient plasmid delivery is often required to generate protective responses in large animals and humans in contrast to mice. Various DNA delivery platforms were developed that demonstrated promising results in large animals and humans, including electroporation (EP) (Sardesai and Weiner, “Electroporation delivery of DNA vaccines: Prospects for success.” Curr. Opin. Immunol. 2011, 23, 421-429), needle free jet-injection (Ault, et al., “Immunogenicity and clinical protection against equine influenza by DNA vaccination of ponies.” Vaccine 2012, 30:3965-3974, Gorres, et al., “DNA vaccination elicits protective immune responses against pandemic and classic swine influenza viruses in pigs.” Clin. Vaccine Immunol. 2011, 18:1987-1995) and lipid deliveries (Sullivan, et al., “A versatile adjuvant for plasmid DNA- and protein-based vaccines.” Expert Opin. Drug Deliv. 2010, 7:1433-1446).

Thus, the inventors contemplate the use of vectors of the present inventions, comprising super-induction sequences, in co-transfection systems with DNA antigen delivery vectors, for enhancing immune protection. In other embodiments, DNA antigen sequences will be cloned into delivery vectors for use with super-induction sequences for enhancing immune protection.

In summary, the technology described herein represents a major advance in solving protein expression problems from existing vectors, particularly when transfecting primary and differentiated cell types. Combinations of gene sequences associated with super-induction of proteins expressed from co-transfected constructs were identified that enhance protein expression using current transfection methods. Therefore, the methods described herein have potential applications in biotechnology and gene therapy for numerous diseases.

D. A Transfection Kit for Providing a Super-Induction Vector of the Present Inventions.

In one embodiment, a kit is contemplated comprising a super-induction plasmid of the present inventions. In a further embodiment, the kit comprises an expression plasmid comprising a promoter and a gene of interest in operable combination, and instructions for co-transfecting the plasmids for super-induction of the gene of interest.

SEQ ID NO:07: pMT2:

AAGCTTTTTGCAAAAGCCTAGGCCTCCAAAAAAGCCTCCTCACTACTTCT
GGAATAGCTCAGAGGCCGAGGCGGCCTCGGCCTCTGCATAAATAAAAAAA
ATTAGTCAGCCATGGGGCGGAGAATGGGCGGAACTGGGCGGAGTTAGGGG
CGGGATGGGCGGAGTTAGGGGCGGGACTATGGTTGCTGACTAATTGAGAT
GCATGCTTTGCATACTTCTGCCTGCTGGGGAGCCTGGGGACTTTCCACAC
CTGGTTGCTGACTAATTGAGATGCATGCTTTGCATACTTCTGCCTGCTGG
GGAGCCTGGGGACTTTCCACACCCTAACTGACACACATTCCACAGGATCC
GGTCGCGCGAATTTCGAGCGGTGTTCCGCGGTCCTCCTCGTATAGAAACT
CGGACCACTCTGAGACGAAGGCTCGCGTCCAGGCCAGCACGAAGGAGGCT
AAGTGGGAGGGGTAGCGGTCGTTGTCCACTAGGGGGTCCACTCGCTCCAG
GGTGTGAAGACACATGTCGCCCTCTTCGGCATCAAGGAAGGTGATTGGTT
TATAGGTGTAGGCCACGTGACCGGGTGTTCCTGAAGGGGGGCTATAAAAG
GGGGTGGGGGCGCGTTCGTCCTCACTCTCTTCCGCATCGCTGTCTGCGAG
GGCCAGCTGTTGGGCTCGCGGTTGAGGACAAACTCTTCGCGGTCTTTCCA
GTACTCTTGGATCGGAAACCCGTCGGCCTCCGAACGGTACTCCGCCACCG
AGGGACCTGAGCGAGTCCGCATCGACCGGATCGGAAAACCTCTCGACTGT
TGGGGTGAGTACTCCCTCTCAAAAGCGGGCATGACTTCTGCGCTAAGATT
GTCAGTTTCCAAAAACGAGGAGGATTTGATATTCACCTGGCCCGCGGTGA
TGCCTTTGAGGGTGGCCGCGTCCATCTGGTCAGAAAAGACAATCTTTTTG
TTGTCAAGCTTGAGGTGTGGCAGGCTTGAGATCTGGCCATACACTTGAGT
GACAATGACATCCACTTTGCCTTTCTCTCCACAGGTGTCCACTCCCAGGT
CCAACTGCAGGCGAGCCTGAATTCGGGGGGGGGGGGGGGGGGGACAGCTC
AGGGCTGCGATTTCGCGCCAAACTTGACGGCAATCCTAGCGTGAAGGCTG
GTAGGATTTTATCCCCGCTGCCATCATGGTTCGACCATTGAACTGCATCG
TCGCCGTGTCCCAAAATATGGGGATTGGCAAGAACGGAGACCTACCCTGG
CCTCCGCTCAGGAACGAGTTCAAGTACTTCCAAAGAATGACCACAACCTC
TTCAGTGGAAGGTAAACAGAATCTGGTGATTATGGGTAGGAAAACCTGGT
TCTCCATTCCTGAGAAGAATCGACCTTTAAAGGACAGAATTAATATAGTT
CTCAGTAGAGAACTCAAAGAACCACCACGAGGAGCTCATTTTCTTGCCAA
AAGTTTGGATGATGCCTTAAGACTTATTGAACAACCGGAATTGGCAAGTA
AAGTAGACATGGTTGGATAGTCGGAGGCAGTTCTGTTTACCAGGAAGCCA
TGAATCAACCAGGCCACCTCAGACTCTTTGTGACAAGGATCATGCAGGAA
TTTGAAAGTGACACGTTTTTCCCAGAAATTGATTTGGGGAAATATAAACT
TCTCCCAGAATACCCAGGCGTCCTCTCTGAGGTCCAGGAGGAAAAAGGCA
TCAAGTATAAGTTTGAAGTCTACGAGAAGAAAGACTAACAGGAAGATGCT
TTCAAGTTCTCTGCTCCCCTCCTAAAGCTATGCATTTTTATAAGACCATG
GGACTTTTGCTGGCTTTAGATCATAATCAGCCATACCACATTTGTAGAGG
TTTTACTTGCTTTAAAAAACCTCCCACACCTCCCCCTGAACCTGAAACAT
AAAATGAATGCAATTGTTGTTGTTAACTTGTTTATTGCAGCTTATAATGG
TTACAAATAAAGCAATAGCATCACAAATTTCACAAATAAAGCATTTTTTT
CACTGCATTCTAGTTGTGGTTTGTCCAAACTCATCAATGTATCTTATCAT
GTCTGGATCCCCGGCCAACGGTCTGGTGACCCGGCTGCGAGAGCTCGGTG
TACCTGAGACGCGAGTAAGCCCTTGAGTCAAAGACGTAGTCGTTGCAAGT
CCGCACCAGGTACTGATATCCCACCAAAAAGTGCGGCGGCGGCTGGCGGT
AGAGGGGCCAGCGTAGGGTGGCCGGGGCTCCGGGGGCGAGGTCTTCCAAC
ATAAGGCGATGATCATCGATGCTAGACCGTGCAAAAGGAGAGCCTGTAAG
CGGGCACTCTTCCGTGGTCTGGTGGATAAATTCGCAAGGGTATCATGGCG
GACGACCGGGGTTCGAACCCCGGATCCGGCCGTCCGCCGTGATCCATGCG
GTTACCGCCCGCGTGTCGAACCCAGGTGTGCGACGTCAGACAACGGGGGA
GCGCTCCTTTTGGCTTCCTTCCAGGCGCGGCGGCTGCTGCGCTAGCTTTT
TTGGCGAGCTCGAATTAATTCTGCATTAATGAATCGGCCAACGCGCGGGG
AGAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAAGG
CGGTAATACGGTTATCCACAGAATCAGGGGATAACGCAGGAAAGAACATG
TGAGCAAAAGGCCAGCAAAAGGCCAGGAACCGTAAAAAGGCCGCGTTGCT
GGCGTTTTTCCATAGGCTCCGCCCCCCTGACGAGCATCACAAAAATCGAC
GCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCT
TACCGGATACCTGTCCGCCTTTCTCCCTTCGGGAAGCGTGGCGCTTTCTC
ATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATC
CGGTAACTATCGTCTTGAGTCCAACCCGGTAAGACACGACTTATCGCCAC
TGGCAGCAGCCACTGGTAACAGGATTAGCAGAGCGAGGTATGTAGGCGGT
GCTACAGAGTTCTTGAAGTGGTGGCCTAACTACGGCTACACTAGAAGAAC
AGTATTTGGTATCTGCGCTCTGCTGAAGCCAGTTACCTTCGGAAAAAGAG
TTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGA
TCCTTTGATCTTTTCTACGGGGTCTGACGCTCAGTGGAACGAAAACTCAC
GTTAAGGGATTTTGGTCATGAGATTATCAAAAAGGATCTTCACCTAGATC
CTTTTAAATTAAAAATGAAGTTTTAAATCAATCTAAAGTATATATGAGTA
AACTTGGTCTGACAGTTACCAATGCTTAATCAGTGAGGCACCTATCTCAG
CGATCTGTCTATTTCGTTCATCCATAGTTGCCTGACTCCCCGTCGTGTAG
ATAACTACGATACGGGAGGGCTTACCATCTGGCCCCAGTGCTGCAATGAT
ACCGCGAGACCCACGCTCACCGGCTCCAGATTTATCAGCAATAAACCAGC
CAGCCGGAAGGGCCGAGCGCAGAAGTGGTCCTGCAACTTTATCCGCCTCC
ATCCAGTCTATTAATTGTTGCCGGGAAGCTAGAGTAAGTAGTTCGCCAGT
TAATAGTTTGCGCAACGTTGTTGCCATTGCTACAGGCATCGTGGTGTCAC
GCTCGTCGTTTGGTATGGCTTCATTCAGCTCCGGTTCCCAACGATCAAGG
CGAGTTACATGATCCCCCATGTTGTGCAAAAAAGCGGTTAGCTCCTTCGG
TCCTCCGATCGTTGTCAGAAGTAAGTTGGCCGCAGTGTTATCACTCATGG
TTATGGCAGCACTGCATAATTCTCTTACTGTCATGCCATCCGTAAGATGC
TTTTCTGTGACTGGTGAGTACTCAACCAAGTCATTCTGAGAATAGTGTAT
GCGGCGACCGAGTTGCTCTTGCCCGGCGTCAATACGGGATAATACCGCGC
CACATAGCAGAACTTTAAAAGTGCTCATCATTGGAAAACGTTCTTCGGGG
CGAAAACTCTCAAGGATCTTACCGCTGTTGAGATCCAGTTCGATGTAACC
CACTCGTGCACCCAACTGATCTTCAGCATCTTTTACTTTCACCAGCGTTT
CTGGGTGAGCAAAAACAGGAAGGCAAAATGCCGCAAAAAAGGGAATAAGG
GCGACACGGAAATGTTGAATACTCATACTCTTCCTTTTTCAATATTATTG
AAGCATTTATCAGGGTTATTGTCTCATGAGCGGATACATATTTGAATGTA
TTTAGAAAAATAAACAAATAGGGGTTCCGCGCACATTTCCCCGAAAAGTG
CCACCTGACGTCTAAGAAACCATTATTATCATGACATTAACCTATAAAAA
TAGGCGTATCACGAGGCCCTTTCGTCTCGCGCGTTTCGGTGATGACGGTG
AAAACCTCTGACACATGCAGCTCCCGGAGACGGTCACAGCTTGTCTGTAA
GCGGATGCCGGGAGCAGACAAGCCCGTCAGGGCGCGTCAGCGGGTGTTGG
CGGGTGTCGGGGCTGGCTTAACTATGCGGCATCAGAGCAGATTGTACTGA
GAGTGCACCATATGCGGTGTGAAATACCGCACAGATGCGTAAGGAGAAAA
TACCGCATCAGGCGCCATTCGCCATTCAGGCTGCGCAACTGTTGGGAAGG
GCGATCGGTGCGGGCCTCTTCGCTATTACGCCAGCTGGCGAAAGGGGGAT
GTGCTGCAAGGCGATTAAGTTGGGTAACGCCAGGGTTTTCCCAGTCACGA
CGTTGTAAAACGACGGCCAGTGCC.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); μM (micromolar); mol (moles); mmol (millimoles); μmol (micromoles); nmol (nanomoles); pmol (picomoles); g (grams); mg (milligrams); μg (micrograms); ng (nanograms); 1 or L (liters); ml (milliliters); μl (microliters); cm (centimeters); mm (millimeters); μm (micrometers); nm (nanometers); ° C. (degrees Centigrade); ° F. (degrees Fahrenheit); Crm1-chromosome region maintenance 1 protein; Env—envelope; ER—Endoplasmic reticulum; HIV-human immunodeficiency virus; HPV—human papillomavirus; HRE—hormone-responsive element; HTLV—human T-cell leukemia virus; LTR—long terminal repeat; LUC—luciferase; MMTV—mouse mammary tumor virus; NC—nucleocapsid; NES—nuclear export sequence; NXF1-nuclear export protein family; NLS—nuclear localization signal; NoLS—nucleolar localization signal; NRE—negative regulatory element; Rem-regulator of export/expression of MMTV mRNAs; RmRE—Rem-response element; RNC—ribosome nascent chain complex; RRE—Rev response element; RxRE—Rex1 response element; SG—stress granule; SP—signal peptide; SRP—signal recognition particle; SU—surface glycoprotein; UPR—unfolded protein response; XBP-1-X—box binding protein-1; cppt—Central polypurine tract; hPGK—Human phosphoglycerate kinase eukaryotic promoter; puroR—Puromycin resistance gene, often used for mammalian positive selection; SIN/LTR-3′ self inactivating long terminal repeat; f1 ori—f1 origin of replication; ampR—Ampicillin resistance gene for bacterial selection; pUC ori—pUC origin of replication; 5′ LTR-5′ long terminal repeat; Psi—retroviral RNA packaging signal.

### Example I

This Example describes exemplary Materials and Methods used herein for making and using co-transfection vectors of the present inventions.

pLKO.1 Plasmids. pLKO.1-control (pLKO.1c), pLK-4250 (shRNA TRCN0000004250), and pLK-4252 (shRNA TRCN0000004252) plasmids were obtained from Sigma. ShRNA hairpin sequences are SEQ ID NO:01: CCGGCAACAAGATGAAGAGCACCAACTCGAGTTGGTGCTCTTCATCTTGTTGT TTTT, SEQ ID NO:02: 5′CCGG-CCTAGCCCTTATTGCATTGTT-CTCGAG-AACAATGCAATAAGGGCTAGG-TTTTT3′, SEQ ID NO:03: 5′CCGG-GATGGATGAATTGCAGTTGTT-CTCGAG-AACAACTGCAATTCATCCATC-TTTTT3′, respectively. Target sequences for pLKO.1-control (pLKO.1c), pLK-4250, and pLK-4252 plasmids are pLK-4250 SEQ ID NO:12: CCTAGCCCTTATTGCATTGTT, pLK-4252 SEQ ID NO:13: GATGGATGAATTGCAGTTGTT (valosin containing protein-VCP), respectively. Valosin-containing protein (VCP) or p97 refers to a type II member of AAA+-ATPase family (ATPase p97 shRNA AAA A), (ATPases with multiple cellular activities) having ubiquitin segregase activity.

Mutant pLKO.1 Plasmids.

LKΔHP was generated by removing the hairpin structure using site-directed mutagenesis. LKΔU6 and LKΔPsi were generated by SDM (site directed mutagenesis) insertion of additional EcoRI sites upstream of the U6 promoter and Psi packaging signal locations, respectively. These SDM products were then digested with EcoRI, and the desired pLKO.1 fragments from which the lentiviral elements were removed were gel-purified and ligated using T4 DNA ligase (Invitrogen).

Additional Plasmids:

pcDNA3 (Life Technologies), ERmCherry, pGL3-control, pHMRluc, pHMARmRERIuc, MigR1 (Addgene. Has an exemplary multiples cloning site for inserting, in one embodiment, a highly structured RNA. In a further embodiment, for inserting a highly structured RNA along with additional super-induction sequences, such as lentiviral sequences of the present inventions. EGFP, and GFP-tagged Rem (GFPRem) (Rem expression plasmids, see, for examples, Byun, et al., J Virol. 86:214 (2012)).

The pLKΔHP, pLKΔU6, and pLKΔPsi were constructed from pLKO.1c during the development of the present inventions. In brief, site-directed insertion of restriction enzyme sites (Stratagene), cleavage, and ligation were used. Any additional references? The pNFκB-GL2 plasmid was kindly provided by Dr. Henry Bose (UTAustin). The pCEP4 plasmid (Life Technologies) and pCEP4 expressing small hairpin (sh)RNAs to either E6AP and a pCEP4 HPV18E6 were a gift from Dr. Jon Huibregtse (UT Austin). The pLL3.7 plasmid was obtained from Addgene, (for example, Plasmid #11795, Empty backbone, lentiviral vector that expresses shRNA under the mouse U6 promoter, a CMV-EGFP reporter cassette is included to monitor expression). The pcDNApsi-sh plasmid was constructed as described herein.

Rem reporter plasmid assays included the Rem-responsive pHMRluc (Renilla luciferase) vector and a second reporter plasmid, pGL3-control (Mertz, et al., “Mouse mammary tumor virus encodes a self-regulatory RNA export protein and is a complex retrovirus.” J. Virol. 79: 14737-14747 2005). The pGL3-control vector lacked the RmRE and expresses firefly luciferase from the SV40 promoter (Promega). The pHMΔeLTR+XRluc constructs with deleted versions of the RmRE were made by insertion of the mutant RmRE (X) into an engineered ScaI site downstream of the splice acceptor site and upstream of the simian virus 40 (SV40) poly (A) signal in pHMΔeLTR1uc (Mertz, et al., “Mouse mammary tumor virus encodes a self-regulatory RNA export protein and is a complex retrovirus.” J. Virol. 79: 14737-14747 2005).

Restriction enzymes were obtained from New England Biolabs (NEB) (Beverly, Mass.) and Invitrogen. Primers were synthesized by Integrated DNA Technologies (IDT) (Coralville, Iowa). After plasmid purification, the deletion and insertion sequences were confirmed using sequence primers in the GFP region. Automated fluorescence sequencing was performed by the DNA sequencing facility at UT Austin's Institute for Cellular and Molecular Biology (ICMB).

### A. Transfection Assays and Reporter Assays.

Tissue culture cell lines (described below) were transfected using different methods (See, 1-4 below) that yielded the highest transfection efficiency. Reporter assays are described below. In general, transfections into human embryonic kidney 293T and 293 cells were performed by the calcium phosphate method, see brief description herein. Each sample had a total of 6 μg DNA adjusted using the empty pcDNA3 vector.

Transfections into Jurkat human T cells were performed using a BTX Electro Cell manipulator as described below. Each sample had a total of 30 or 42 μg of DNA adjusted with pcDNA3. Rat XC fibroblast transfections were used with the DMRIE-C reagent and a total of 6 μg of DNA. DNA samples for transfection were highly purified to remove contaminants using cesium chloride gradients. Transfections were performed in triplicate.

Reporter activities were determined by the Dual Luciferase system (Promega) and reported per 100 μg of protein. The results are given as the mean and standard deviation relative to samples containing the empty pcDNA3 vector and the reporter vector. Experiments were performed at least twice with similar results.

1. DMRIE-C Transfection.

XC cells were harvested the day before the transfection and counted using a Bright-line hemacytometer (Reichert, Buffalo, N.Y.). Cells (5×10⁵) were plated in each well of a 6-well tissue culture (TC) plate in 2 ml of tissue culture medium. Different concentrations of plasmid DNA were mixed in 0.5 ml of transfection medium (tissue culture medium without serum and antibiotics) in a 1.7 ml microcentrifuge tube. In a separate tube, 10 μl of DMRIE-C transfection reagent (Invitrogen) was mixed with 0.5 ml of transfection medium. The DNA/medium mixture was added to the DMRIE-C/medium in a 1:1 ratio and incubated at RT for 45 min. The plated XC cells were washed with transfection media, and 1 ml of the transfection mixture was added to the well. Plates were then returned to the 7.5% CO₂ environment and incubated for 6 to 8 h at 37° C. The transfection was terminated by adding 1 ml of complete medium containing twice the normal concentration of FBS without removing the DNA-containing medium. Cells were incubated at 37° C. in a CO₂ incubator for a total of 48 hour.

2. Electroporation.

Jurkat T cells and HC11 mammary epithelial cells were transfected by electroporation using a BTX Electro Cell Manipulator (BTX, San Diego, Calif.). On the day prior to transfection, Jurkat cells were replated at 1.2×10⁷ cells in 20 ml of complete growth media. On the day of transfection, 1×10⁷ cells were mixed with 20 to 40 μg of total DNA in 400 μl of serum-free RPMI. The Jurkat cell/DNA mixture was added to a 4 mm gap cuvette (BTX) and electroporated using the following parameters: 260V, 1050 μF, and R10 (720Ω). After a 10-min incubation at RT, transfected Jurkat cells were replated in 60 mm dishes in 4 to 5 ml of complete medium. After a 48 hr incubation, cells were harvested for further assays.

HC11 cells were treated with trypsin and diluted 1:3 from a confluent 100 mm plate on the day before transfection. On the day of transfection, 1×10⁷ cells were mixed with 15 to 20 μg of total DNA in 200 μl of serum-free RPMI. Using a 2 mm gap cuvette, the HC11 cell/DNA mixture was subjected to electroporation using the following optimized conditions: 140V, 1750 μF, and R4 (72Ω). After a 10-min incubation at RT, transfected HC11 cells were replated in 60 mm TC plates in 4 to 5 ml of complete medium. After a 48 h incubation, cells were harvested for further assays.

3. Calcium Phosphate Precipitation.

The 293 and 293T cells were transiently transfected by the calcium phosphate method. One day prior to transfection, cells (5×10⁵) were plated in 6-well TC plates in 2 ml of complete medium. For the transfection, DNA (6 μg total) was mixed with 10 μl of CaCl₂ (final concentration of 0.25 M) in a 1.7 ml microcentrifuge tube. The volume was adjusted to 100 μl with H₂O and added dropwise to a second tube containing 100 μl of 2×-HBS, pH 7.1 or 7.2 (280 mM NaCl, 10 mM KCl, 1.5 mM Na₂HPO₄.2H₂O, 12 mM dextrose, 50 mM HEPES) while vortexing. The mixture was incubated at RT for 15 min and added to the cells dropwise. At 6 to 8 h post-transfection, the media was changed to remove the calcium phosphate precipitate. Cells were harvested after 48 h.

4. Lipofectamine 2000 Transfection.

In some cases, 293 cells were also transiently transfected using Lipofectamine 2000 Reagent (Invitrogen). The day before transfection, 5×10⁵ cells were seeded per well of a 6-well plate in 2 ml of complete medium without antibiotics. For each transfection, a total of 6 μg of DNA was added to 250 μl DMEM without additives. In a separate tube, 5 μl of Lipofectamine 2000 Reagent was diluted in 250 μl DMEM containing 4% FBS without antibiotics. The DNA/medium mixture then was added to the Lipofectamine 2000/medium at a 1:1 ratio and incubated at RT for 20 min. The DNA-Lipofectamine 2000 Reagent complexes were added to cells dropwise. Cells were harvested after a 48 h incubation.

B. Reporter Gene Assays.

The dual-Luciferase Reporter Assay System (Promega, Madison, Wis.) was used to detect luciferase reporter gene activity. Transfected cells were harvested, washed with 1×PBS, and resuspended in 1× Passive Lysis Buffer (Promega). Subsequently, three cycles of freezing at −80° C. for 20 min and thawing in a 37° C. water bath were performed. The lysates were centrifuged at 8,000 rpm in a Beckman Coulter Microfuge 18 Centrifuge for 10 min at 4° C., and the supernatant was transferred to another tube. The Bio-Rad Protein assay system was used to quantitate protein concentration. A total of 40 μg or 20 μl of lysate was used to obtain readings for both firefly and Renilla luciferase using a Turner TD-20e luminometer (Turner Designs, Inc., Sunnyvale, Calif.) that was set to 0 delay and 10 sec integration. Luciferase Assay Reagent II (LAR II) (Promega) (50 μl) was added to the sample, and the firefly luciferase activity was determined. The Renilla luciferase activity was determined after the addition of 50 μl Stop and Glo Reagent (Promega). The luciferase was expressed in relative light units (RLUs), and the relative values were normalized to 100 μg of protein.

C. Cell Lines.

XC rat fibroblast cells transformed by Rous sarcoma virus were grown in Dulbecco's modified Eagle's medium (DMEM) (Invitrogen, Carlsbad, Calif.) supplemented with 5% heat-inactivated (30 min at 56° C.) fetal bovine serum (FBS) (Invitrogen), 100 Units/ml penicillin, 100 μg/ml streptomycin (Invitrogen), 2 mM L-glutamine and 50 μg/ml gentamycin sulfate (Elkins-Sinn, Inc., Cherry Hill, N.J.). Jurkat human T lymphoma cells were maintained in suspension using Roswell Park Memorial Institute (RPMI) 1640 medium (Invitrogen) containing 5% FBS, 100 Units/ml penicillin, 100 μg/ml streptomycin, 2 mM L-glutamine and 50 μg/ml gentamycin sulfate. HC11 normal mouse mammary epithelial cells were grown in RPMI 1640 medium supplemented with 10% FBS, 0.5 μg/ml insulin (Sigma, St. Louis, Mo.), 0.5 μg/ml epidermal growth factor (Invitrogen), 2 mM L-glutamine and 50 μg/ml gentamycin sulfate. Human embryonic kidney cells (293) were maintained in DMEM containing 10% FBS, 100 Units/ml penicillin, 100 μg/ml streptomycin, 2 mM L-glutamine and 50 μg/ml gentamycin sulfate. Cell lines were maintained at 37° C. in a humidified atmosphere containing 7.5% CO₂.

### D. Western Blotting and Antibodies.

Transfected cells were used for preparation of whole cell extracts by the addition of one volume of 2×SDS-containing gel electrophoresis loading buffer. Up to 100 μg of cell extracts were resolved in 10-12% polyacrylamide gels containing 1% SDS and transferred to nitrocellulose membranes. Membranes were incubated with various antibodies for 1-2 hr, washed, and incubated with horseradish peroxidase-tagged secondary antibodies for 1-2 hr. Antibody binding was detected using the Western Lightning Enhanced Chemiluminescent Reagent (Perkin Elmer). Antibodies were obtained from the following sources: GFP (Clontech), β-actin (ChemBioChem), GAPDH (Cell Signaling Technology), B23 (Sigma-Aldrich), phosphorylated eIF2α (Cell Signaling Technology), BiP (BD Bioscience/BD Transduction Laboratories). Antibodies to G3BP were generously provided by Dr. Richard Lloyd (Baylor College of Medicine).

### E. RT-PCR.

Total RNA was extracted by the guanidinium isothiocyanate method and used for semi-quantitative RT-PCR. Equal amounts of cDNA were used for incubation with primers for Renilla luciferase, gapdh or rem (rem (gapd427+/983−; Remp71L+/RemΔ100−; Rluc 1409+/1904−) Rluc1409+: 5′ GATTGGGGTGCTTGTTTGG3′ [SEQ ID NO:14], R1uc1904−: 5′ TTCCCATTTCATCAGGTGC3′ [SEQ ID NO:15], gapd427+: 5′ CATGTTTGTGATGGGTGTGAACCA3′[SEQ ID NO:16], gapd983−: 5′ GTTGCTGTAGCCGTATTCATTGTC3′[SEQ ID NO:17], RemP71L+: 5′ AAGCGGAGGAGGTTCTTAAAACCTCACAAAC3′ [SEQ ID NO:18], RemΔ100−: 5′ GGATCCCTACCACATACGCCTTTTATCACC3′ [SEQ ID NO:19]), and the products were analyzed on 1% agarose gels and stained with ethidium bromide.

### F. Fluorescent Microscopy and FACS Analysis.

Cells were plated on glass coverslips in 6-well tissue culture plates approximately 48 hr post-transfection and fixed in 4% paraformaldehyde for 15 min, and then permeabilized with 0.05% Triton X-100 for 30 min at room temperature. After three 15 min washes, cells were incubated with a blocking solution containing 2% fetal bovine serum, washed, and then incubated with G3BP-specific antibody for 1 hr at 37° C.

After three washes in PBS, cells were incubated with goat anti-rabbit Alexafluor 594 (Invitrogen) (1:500) at 37° C. in the dark. Cells were washed thrice in PBS and stained with 150 nM 4′6-diamidino-2-phenylindole (DAPI) in the dark. After three additional PBS washes, coverslips were mounted on glass slides in VECTASHIELD (Vector Labs), sealed, and examined on an Olympus IX70 microscope. FACS analysis was performed on duplicate transfections using the BD LSRII Fortessa Cell Analyzer (BD Biosciences).

### G. Pulse Labeling.

Cells (293) were transfected as described above, and the media was changed after 7 hour. At 48 hr post-transfection, cells were incubated in Dulbecco's lacking cysteine and methionine for 30 min followed by a 30 min pulse in the same media containing μCi/ml of ³⁵S methionine/cysteine (Perkin-Elmer). Cells were treated with cold 10% TCA, and the precipitates were washed twice with 10% TCA. The acid was neutralized with 1N NaOH, and the radioactive incorporation was measured by scintillation counting.

### H. Drug Inhibition Experiments.

The Mnk1 inhibitor (Calbiochem) was added at 2 nM for 40 hr, and the p38 inhibitor (SB203580) was added at 10 μM for 24 hr prior to harvesting transfections.

### I. Statistical Analysis.

Transfection results are given as the mean±standard deviation of triplicates. Results were compared by the two-tailed Student's t test with a P value of <0.05 considered to be significant.

### Example II

This Example describes exemplary Materials and Methods showing the surprising result of super-induction of Rem in a co-transfection system with a lentiviral plasmid expected to at least restore wild-type Rem activity.

While studying the effect of silencing p97/VCP on Rem retrotranslocation, pLKO.1-based shRNAs vectors containing shRNAs against p97, and a control shRNA plasmid (see, exemplary pLKO.1-c in FIG. 1B), were purchased and tested for their effectiveness in knocking down the level of p97/VCP. In silencing assays, #4250 transfection showed a measurable decrease in p97 protein levels in comparison to #4252 and pLKO.1-puro control. A luciferase value of reporter vector co-transfected with an empty vector was set to 1. These plasmids were then tested in co-transfection experiments in 293T cells with a GFPRem expression plasmid for measuring their effect upon the activity of mouse mammary tumor virus (MMTV) Rem, a Rev-like RNA-binding protein. When these experiments instead showed super-induction of Rem by pLKO.1-#4250 shRNA, pLKO.1-#4252 shRNA and even with the pLKO.1 control shRNA plasmid (FIG. 1D), the inventors set up co-transfection experiments as described in the EXAMPLES below to directly observe this unexpected super-induction effect.

### Example III

This Example describes exemplary Materials and Methods showing additional surprising results of super-induction of Rem and other co-transfected genes on expression plasmids in a co-transfection system. Rem activity was measured by co-transfection of the pHMRluc plasmid which requires Rem binding to the Rem-responsive element in the Renilla luciferase transcript.

Further experiments demonstrated that pLKO.1 induced elevated expression of luciferase genes in co-transfected reporter plasmids, pGL-3 control expression plasmid and pHMRluc Rem responsive expression plasmid in 293T cells (FIG. 1E). Different shRNAs of pLKO.1 plasmids, including the control shRNA with no known cellular target (pLKO.1c-control/pLKO.1c), showed a similar effect. The co-transfected reporter plasmids expressed different reporter genes (i.e. firefly luciferase or a Rem responsive Renilla luciferase) from either the SV40 (pGL-3 control) or CMV (pHMRluc) promoters, respectively. The expression of luciferase from pGL-3 control and pHMRluc reporters were elevated approximately 2-to-6-fold in the presence of pLKO.1c, whereas the co-transfection of an additional Rem expression vectors elevated Renilla luciferase activity approximately 10- to 30-fold in the presence of pLKO.1 plasmids. Increased Rem production of approximately 5-fold also was detected by Western blotting (FIG. 1F).

pLKO.1c was then co-transfected with either the EGFP or ER-mCherry expression vectors. Levels of induced protein then were monitored by fluorescence microscopy (FIGS. 2A-F and 2G-I). The data showed that both fluorescent proteins were induced by the presence of pLKO.1c in trans, consistent with findings from the reporter assays and Western blotting for MMTV Rem (FIG. 10). Similar transfections were analyzed by FACS analysis (FIGS. 2J&K). At lower levels of transfected DNA, pLKO.1c or the shRNA-deleted version increases fluorescence by at least 2-to-3-fold. Together with previous experiments, these results indicated that expression of different exogenous proteins was enhanced by co-transfection with pLKO.1c. Furthermore, pLKO.1c affected exogenous proteins that were localized to different cellular compartments since ER-mCherry is primarily in the endoplasmic reticulum, whereas EGFP diffuses throughout the cells.

Other lentiviral shRNA expression vectors were tested for super-induction effects. The 293T cells were co-transfected with a Rem reporter and expression vectors in the presence of pLKO.1c or pLL3.7 (FIG. 3A). The pLL3.7 and pLKO vectors induced basal Renilla luciferase activity from pHMRluc up to 3-fold in the absence of Rem (FIGS. 3B&C), whereas both vectors induced Renilla activity from 11-to-13-fold in the presence of Rem. Compared to pLKO.1c, pLL3.7 had little effect on GFP-SP expression by Western blotting (FIG. 3D). Co-transfection of the pLL3.7 vector also increased activity from the pGL3-control vector up to approximately 2-fold. Thus, although the shRNA hairpin was a major determinant of increased expression in the presence of pLKO.1, other sequences in the lentiviral vector influenced expression from co-transfected vectors.

Experiments with another gammaretroviral vector (pSuper.retro.puro) showed that co-transfection of a gammaretroviral vector increased exogenous protein expression in trans. In particular, co-transfection with pSuper.retro.puro induced Rem and reporter gene expression approximately 2-fold.

The gammaretroviral vector MigR1 (FIG. 5A) was co-transfected together with a Rem expression and reporter vectors in both 293 and 293T cells. In 293 cells, the firefly luciferase value from the co-transfected pGL-3-control vector was induced 5.6-fold by pLKO.1c, however, MigR1 slightly increased reporter values by around 1.5-fold (FIGS. 5B&C). Transfection of pLKO.1c resulted in a 5-fold increase in Renilla luciferase activity in the absence of Rem, whereas MigR1 gave an approximately 3-fold increase. In contrast, inclusion of the plasmid-based vector, pEGFP-N3 (Clontech), decreased both Renilla and firefly luciferase activities. Rem expression induced Renilla reporter values approximately 29-fold and was further induced approximately 11-fold by the addition of pLKO.1c and 4.5-fold by MigR1.

Western blotting confirmed that Rem, but not actin, expression increased in the presence of pLKO.1c (FIG. 5F). Since the MigR1 retroviral vector encodes the heterologous GFP protein from a highly structured RNA region (IRES), a deletion of these sequences was performed to determine the effect on induction of co-transfected genes. Co-transfection of either MigR1 or its deleted derivative MigΔIRES-GFP produced very similar increases in firefly luciferase reporter activity in 293T cells (FIG. 5E) yet increased expression of Renilla luciferase from pHMRluc. MigΔGFP had no effect on GFPRem levels (FIG. 5F).

A similar trend was observed in 293T cells with these two retroviral vectors inducing Renilla luciferase, whereas the plasmid-based vector was inhibitory. These results are consistent with effects of retroviral vectors on both Rem expression and luciferase reporter levels to super-induced Renilla expression.

Surprisingly, MigR1 had larger effects on pGL3-Control firefly luciferase expression compared to pHMRluc Renilla luciferase, whereas pLKO.1c had the opposite result, compare relative expression shown in FIG. 5D with FIG. 5E. These data support that cis-acting sequences in gammaretroviral genomes induced increased expression of co-transfected genes in trans.

A. Super-Induction in Other Cell Types and Using Additional Transfection Procedures.

Although the effect was smaller, pLKO.1c also increased expression of pHMRluc in the presence or absence of the RmRE in XC rat fibroblast cells transfected by a lipid-based method (FIGS. 1G&H) and increased Rem levels (FIG. 1I).

Jurkat human T cells were transfected by electroporation with similar results (FIGS. 9A&B), indicating that pLKO.1c super-induction was not cell-type, species or method specific. The increase in pHMRluc activity observed after co-transfection with pLKO.1c was independent of the presence of the Rem-responsive element (RmRE) and dependent upon Rem (FIGS. 1J&K). Samples received the same total amount of DNA (6 μg), indicating that different DNA levels were not responsible for changes in transfection efficiency and protein expression. Therefore, the higher induction in the presence of Rem was likely due to stimulation of both basal Rem levels and Renilla luciferase values.

B. Super-Induction was not Observed for Endogenous Genes.

Examination of total protein levels by Coomassie staining and by incubation of Western blots with antibody specific for endogenous actin (FIG. 1F) did not reveal detectable differences in endogenous protein production. A similar result was obtained when pLKO.1c expressed a control shRNA or an shRNA specific for the AAA ATPase, p97, although 2-to-3-fold differences were observed (FIG. 1F). These results suggested that the pLKO.1c vector increased expression from exogenously expressed genes in trans.

### Example IV

This Example describes exemplary Materials and Methods showing the contribution of shRNA and lentiviral genes (and a lentiviral unit) to super-induction through mutation analysis.

Co-transfection with a lentivirus vector expressing a non-specific shRNA led to increased protein expression in a trans-activating system, whereas the pCEP4 non-lentiviral vector revealed firefly luciferase activity different from pLKO.1. Thus deletion mutants were made for detetmining the contribution of lentiviral elements, shRNA element, and pLKO.1 sequences to define their potential effect on activity compared to full-length pLKO.1. shRNA-expressing lentiviral containing plasmids based on the pLKO.1 vector (Sigma) (FIG. 1L) were used to determine the relative contribution of the shRNA and lentiviral sequences.

A. Determinants of pLKO.1c-mediated protein induction.

To investigate the pLKO.1c sequences needed for the increased protein levels, the shRNA hairpin (AHP) mutant was constructed and used for co-transfection with both Rem-expression and Rem-responsive vectors. The 293 cells were transiently co-transfected with 5 μg of the deletion mutants of pLKO.1 in the presence and the absence of 12.5 ng of the GFPRem expression construct. The same amount of pLKO.1 and LKΔHP were co-transfected with the GFPRem expression plasmid separately for comparison of their activity with that of the deletion mutants.

The results from these experiments indicated that removal of the hairpin decreased the level of Rem expression, however the amount of Rem exceeded that obtained in the absence of the pLKO.1c vector as determined both by reporter assays and Western blots (FIGS. 1M&N and FIG. 10). The LKΔU6 and LKΔPsi mutants also increased the expression levels of Rem. Increased Rem levels were observed when GFPRem expression vector was co-transfected with either pLKO.1 or the deletion mutants, as observed in Western blotting of the cell extracts from the same transfection. Note that this highly sensitive reporter assay is saturable at relatively low Rem levels (Byun et al., “Retroviral Rem protein requires processing by signal peptidase and retrotranslocation for nuclear function.” Proc Natl Acad Sci USA. 107(27):12287-92 (2010)), but luciferase activity is still approximately 5-fold higher in cells transfected with the hairpin-deleted form of pLKO.1c. Similar experiments were performed by co-transfection of pLKO.1c with an HTLV Rex expression vector together with a Rex-responsive plasmid (Mertz, et al., “Rev and Rex proteins of human complex retroviruses function with the MMTV Rem-responsive element.” Retrovirology, 6:10 (2009). Results were comparable to those observed with Rem and its cognate reporter vector.

LKΔPsi co-transfection had the least effect on Rem levels compared to the other pLKO.1-based plasmids, however levels were higher than Rem alone. LKΔHP and LKΔU6 had similar effects on Rem protein levels. Cells from the same transfection were observed under a fluorescence microscope. Co-transfection of the GFPRem expression construct with these pLKO.1 deletion mutants revealed an increased GFP signal intensity compared to the GFPRem construct alone, but the intensity was less than that of the co-transfection of the GFPRem plasmid with pLKO.1. This observation confirms that the lentiviral element of pLKO.1 (tested using LKΔPsi) affects Rem activity and expression levels to some degree, but does not abolish super-induction completely.

These experiments suggest that the structured shRNA in pLKO.1c contributes significantly to exogenous protein expression, although other sequences also contribute.

B. Effect of shRNA expression in the absence of lentiviral sequences. pLKO.1 is a lentiviral vector containing part of the HIV genome, including the 5′ LTR, the 5′ UTR, the 5′-end of the gag gene, the RRE, part of the env gene and the 3′ LTR. To test the effect of shRNA expression in the absence of lentiviral sequences, Rem expression and reporter vectors were co-transfected together with a non-lentiviral shRNA expression vector. pCEP4 (Invitrogen) is an episomal mammalian expression vector that does not contain lentiviral elements.

A shRNA vector based on pCEP4 was developed by inserting a histone H1 polymerase III promoter followed by a hairpin into the pCEP4 vector. The pCEP4 control vector and two of the pCEP4-based hairpin-containing vectors, E6AP-3 and HPV18E6 (kindly provided by the Huibregtse laboratory), were co-transfected with GFPRem expression plasmids into 293 cells. pLKO.1 was independently co-transfected with GFPRem as a control. The pCEP4 vector decreased both firefly and Renilla luciferase in the presence or absence of Rem. The presence of either shRNA increased activity over that of pCEP4.

Unlike pLKO.1, pCEP4 slightly decreased Renilla reporter expression in the presence or absence of Rem with larger (up to 10-fold) decreases on firefly reporter activity, which lacks the Rem-responsive element (FIGS. 3E&F). pCEP4 vectors were tested expressing two different shRNAs expressed from the H1 promoter. The pCEP4 vector carrying either shRNA gave 3-to-4-fold increases in Renilla luciferase levels in the absence of Rem. The pCEP4 vector carrying shRNA#1 specific for E6AP had less than a 2-fold effect on Rem-induced expression, whereas shRNA#2 specific for HPV18E6 (not present in these cells) gave approximately 6-fold super-induction in the presence of Rem, comparable to that observed with pLKO.1c, which expresses a control shRNA.

Together, these experiments showed that sequences derived from shRNAs as well as lentiviruses contribute to maximal induction of co-expressed genes by pLKO.1c. Since pLKO.1 also increased the activity of Rev and Rexl, Rev was co-transfected in 293 cells with either pLKO.1 or LKΔHP to determine the effect of the hairpin deletion on Rev activity. In this experiment, pLKO.1 caused a slight (3-fold) increase in the basal activity of both reporter vectors, pRRE-Rluc and pHMRluc, but LKΔHP did not increase the levels to the same degree. This moderate increase in activity was similar to that observed with Rem. Therefore, the deletion of the hairpin in pLKO.1 resulted in a significant decrease in the super-induction of both Rem and Rev, with LKΔHP showing some residual induction.

### Example V

This example describes exemplary Materials and Methods for providing a super-induction vector of the present inventions by subcloning the super-induction unit, i.e. sequences from the pLKO.1c.

Based on these results described here, a portion of pLKO.1c containing the packaging signal yr, the 5′ UTR, the Rev-responsive element (RRE), as well as the U6 promoter upstream of the shRNA into the multiple cloning site of plasmid vector, pcDNA3 (Invitrogen) (FIG. 4A). This new construct, pcDNApsi-sh, was co-transfected into 293 cells with both Rem-responsive and non-responsive vectors in the presence and absence of Rem expression. Interestingly, co-transfection of either pcDNApsi-sh or pLKO.1c increased Renilla luciferase expression about 4-fold in the absence of Rem (FIGS. 4B&C).

A 4- to 5-fold elevation of the co-transfected pGL-3-control plasmid also was observed in the presence of either pcDNApsi-sh or pLKO.1c. Addition of the Rem expression plasmid increased Renilla luciferase activity by approximately 13-fold over that observed in the absence of Rem. As expected, firefly luciferase expression was not responsive to Rem.

Western blotting of extracts from these transfections indicated that pcDNA3psi-sh increased Rem expression, but not to same level as pLKO.1c (FIG. 4D). These experiments suggested that transfected DNA vectors that express highly structured RNAs may lead to increased expression of co-transfected genes in trans.

To determine whether pLKO.1c induced changes in the transcription of co-expressed genes, transfection experiments with Rem expression and reporter vector were performed in the presence and absence of pLKO.1c. After 48 hr, cells were extracted for RNA, and RNA samples were used for semi-quantitative RT-PCR with primers specific for rem or Renilla luciferase. The results indicated that pLKO.1c did not affect the steady state level of rem or Rluc mRNAs (FIG. 4E) when protein levels increased (FIG. 4F). RT-PCRs specific for the endogenous housekeeping gene, gapdh, confirmed that uniform levels of cDNA were used for these reactions. Together with previous results, these experiments suggest that the introduction of a subset of sequences in pLKO.1c in multiple cell types leads to increased translation of genes introduced simultaneously on different vectors.

### Example VI

This example describes exemplary Materials and Methods for providing an adenovirus based super-induction vector of the present inventions by inserting VA-RNA (forming miRNAs or mivaRNA) highly structural RNA sequences.

In a co-expression experiment, a pMT2 adenoviral gene containing plasmid on a pBR322 backbone, expressing an Ad VA-RNA sequence, see FIG. 16, was compared to a pMT2deltaVA mutant without the majority of the VA sequences, see FIG. 17, a pLKO.1c super-induction plasmid, and a pcDNA3 control. Briefly, 2.5 ng of an EGFP expression plasmid was co-transfected with each one of the constructs listed below the image in FIG. 15. Surprisingly, super-induction of GFP by VA-RNA was observed comparable to super-induction of GFP by pLKO.1e. This super-induction effect was absent when the pMT2deltaVA mutant without the majority of the VA sequences was co-transfected with the EGFP expression plasmid. Thus, in addition to shRNA, VA-RNA finds use as a super-induction sequence of the present inventions.

### Example VII

This example shows that pLKO.1 super-induction did not influence expression of endogenous cellular genes. Further, pLKO.1c co-transfection did not affect levels of co-transfected proteins through eIF-2α phosphorylation, interferon signaling or the unfolded protein response. In other words, induction of protein expression did not affect eIF-2α phosphorylation.

Infection with single-stranded RNA viruses often leads to detection of double-stranded RNA structures and signaling through type I interferons (Malireddi and Kanneganti, “Role of type I interferons in inflammasome activation, cell death, and disease during microbial infection.” Front Cell Infect Microbiol. 3:77 (2013)). Double-stranded RNAs activate RNA-dependent protein kinase (PKR), which phosphorylates the translation initiation factor, eIF-2α (Yim and Williams, “Protein kinase R and the inflammasome.” J Interferon Cytokine Res. 34(6):447-54 2014). Phosphorylated eIF-2α prevents the formation of translation initiation complexes, leading to reduced translation within infected cells. To determine if the presence of pL1(0.1c resulted in changes in eIF-2α phosphorylation, a co-transfection was repeated using MMTV Rem expression vectors. Protein extracts from transfected cells were tested after Western blotting with specific antibodies (FIG. 6A). Increased GFP-tagged Rem levels were observed in the presence of pLKO.1c (FIG. 6A, compare lanes 3 and 4). Additional Western blots indicated that transfection with plasmid pcDNA3 increased the level of phosphorylated eIF-2α compared to mock-transfected cells (FIG. 6A, compare lanes 1 and 5). Actin levels showed equal loading of protein extracts. These results indicated that certain transfection conditions may reduce cellular translation initiation.

Endogenous genes, such as BiP, B23 or GAPDH, which increase in expression during an unfolded protein cell response and/or viral infections were chosen for observation after cells were co-transfected with a LKO.1 plasmid and an expression plasmid with a recombinant gene. However, Western blotting of extracts from these transfected cells revealed little difference in BiP, B23 or GAPDH levels (FIG. 6B). Therefore, activation levels of other endogenous genes were measured, such as phosphorylation of eIF-2α. However, eIF-2α phosphorylation did not change in the presence of pLKO.1c. Furthermore, Western blotting revealed no differences of the interferon-induced protein, RIG-G, after introduction of pLKO.1c (FIG. 6C).

A general indicator of cell stress is activation of the transcription factor, nuclear factor of kappa beta (NF-κB). NF-κB exists as an inactive form in the cytoplasm, but signaling through multiple cellular pathways leads to the degradation of the associated inhibitor IκB. Nuclear translocation of NF-κB leads to DNA binding, altered gene expression, and either stress remediation or apoptosis. To determine whether pLKO.1c affected NF-κB activity, pLKO.1c plasmid was co-transfected together with the NF-κB-reporter plasmid, NF-κB-GL2. Extracts from control transfections indicated that both GFPRem and pLKO.1c increased pHMRluc activity (FIG. 10A).The presence of pLKO.1c and Rem decreased firefly luciferase levels approximately 3-to-4-fold (FIG. 10B). Co-transfection of pLKO.1c reduced Rem-mediated Renilla luciferase activity by 2-fold (FIG. 10A). These results indicated that pLKO.1c has a general inhibitory effect on reporter plasmid vectors, such as pGL3-control and NFκB-GL2.

### Example VIII

This example shows that pLKO.1 super-induction reduced expression of stress granules.

The stress-granule proteins TIA-1 and G3BP affect pLKO.1 super-induction of Rem activity. Some positive-stranded RNA viruses, such as poliovirus, are known to increase translation of their mRNAs by inhibition of stress granule formation (Reineke and Lloyd, “Diversion of stress granules and P-bodies during viral infection.” Virology, 20; 436(2):255-267(2013)). Stress granules are believed to be aggregations of translation initiation factors and translation-arrested mRNAs in addition to other proteins, e.g., Ras GAP-SH3-binding protein (G3BP), T-cell intracellular antigen 1 (TIA-1), and TIA-1 related protein (TIAR). These granules accumulate in the presence of specific cellular stresses, such as viral infection. To determine if pLKO.1 affects the formation of stress granules, co-transfections with this lentiviral vector were performed in the presence and absence of Rem. Cells were transfected with TIA-1-YFP or G3BP-YFP in the presence or absence of Rem. GFPRem expression vector (12.5 ng) and pLKO.1 (5 μg) were co-transfected into 293 cells. Both GFPRem expression plasmid and pLKO.1 were transfected independently as controls. Mock-treated cells (without DNA) were included as a control for stress due to the transfection process.

After 48 hr, cells were fixed, permeabilized, incubated with antibody specific for G3BP, and examined by fluorescence microscopy. Alexafluor 594-conjugated rabbit antibody was used to detect the G3BP-bound primary antibody. Cells were stained with DAPI for nuclear visualization.

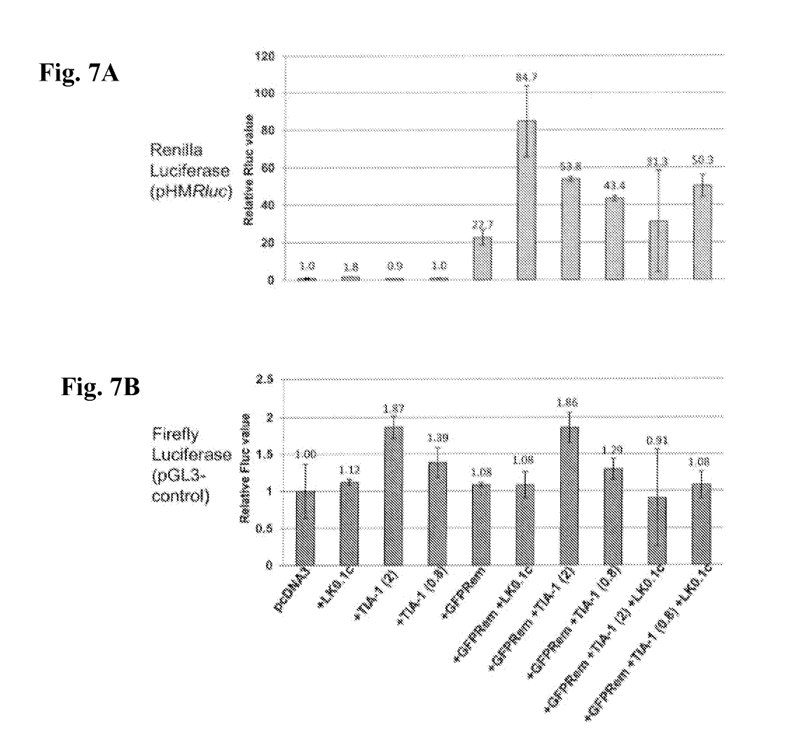

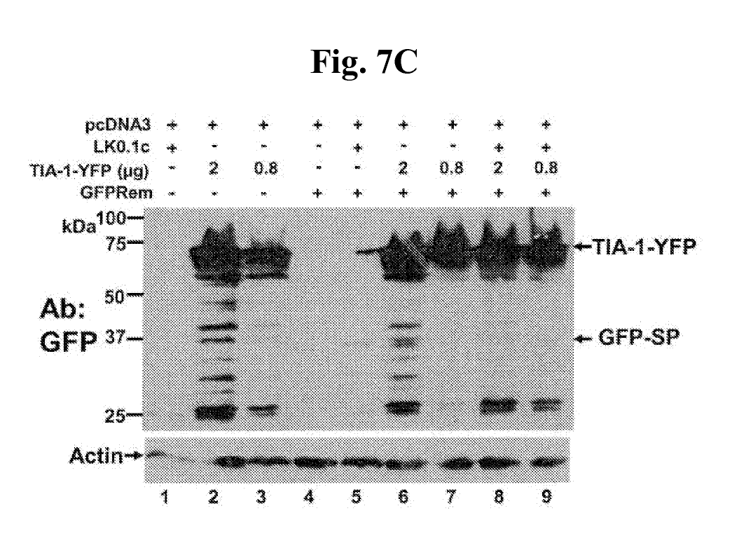

TIA-1 had little effect on basal Renilla or firefly reporter levels (FIGS. 7A&B). Co-transfection with Rem gave approximately a 23-fold induction of Renilla luciferase activity, and addition of pLKO.1c gave a further 4-fold increase. Surprisingly, TIA-1, but not G3BP, increased Rem activity on pHMRluc reporter approximately 2-fold. Co-transfection of TIA-1 with pLKO.1c gave Renilla values comparable to TIA-1 alone. Western blotting confirmed high levels of TIA-1 expression (FIG. 7C). pLKO.1c-induced GFP-SP expression, whereas TIA-1 expression slightly interfered with pLKO.1c-mediated Rem induction. TIA-1 alone had no apparent effects on Rem levels in the absence of pLKO.1c. (FIG. 7C, lane 7).

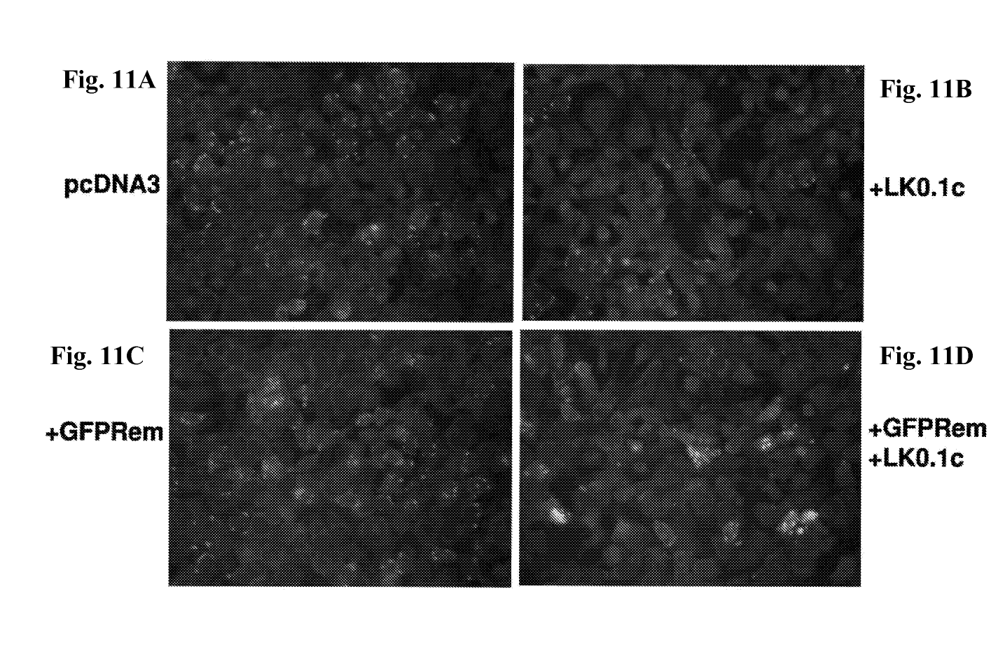

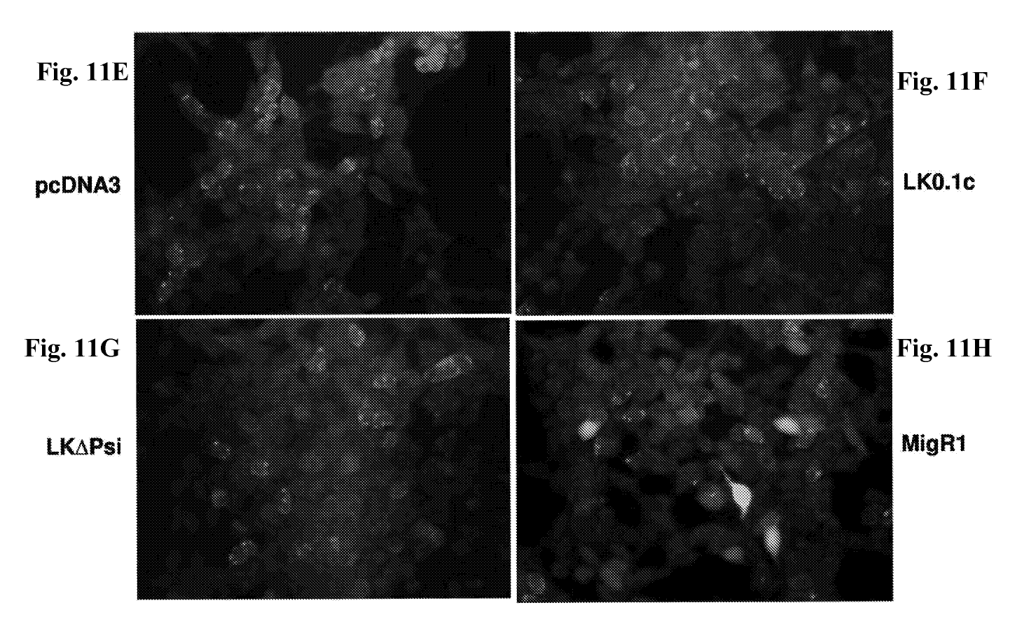

Cells transfected with pLKO.1c in the presence or absence of Rem had fewer stress granules (FIG. 11A-D). Additional results suggested that transfection of certain vectors, particularly pcDNA3, could induce stress granules, but that these granules did not correlate with the observed super-induction of co-transfected reporter genes (FIG. 11E-H).

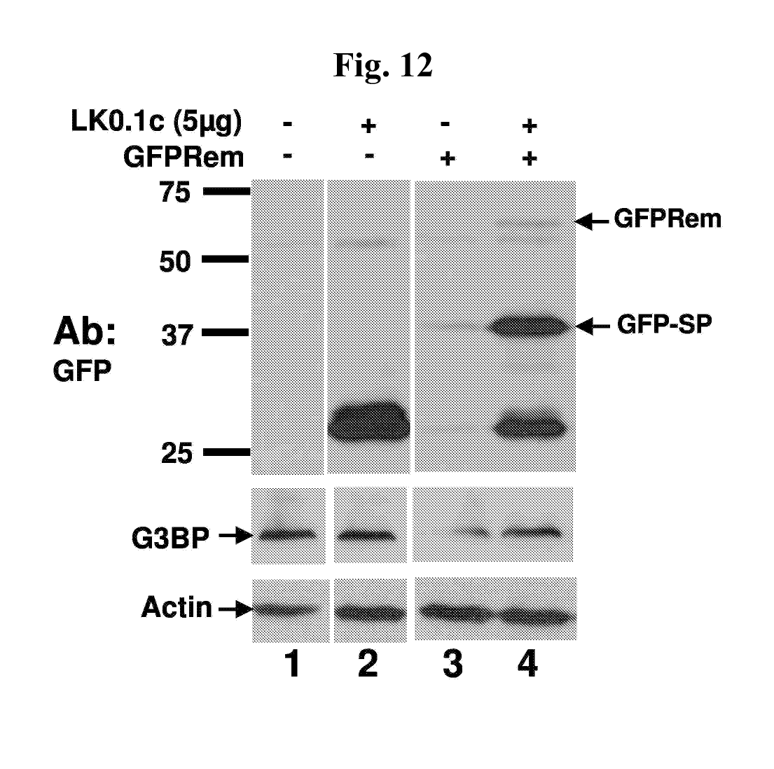

Poliovirus is known to relieve translational arrest after cleavage of G3BP by virally encoded protease. To test whether pLKO.1c altered levels of G3BP, co-transfected cells were used for preparation of cell extracts and Western blotting with G3BP-specific antibodies. The results indicated that G3BP levels and phosphorylation were unchanged (FIG. 12).

### Example IX

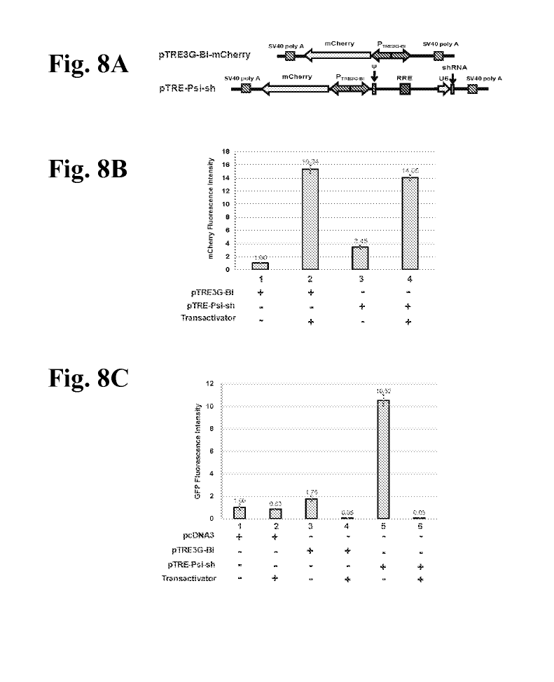

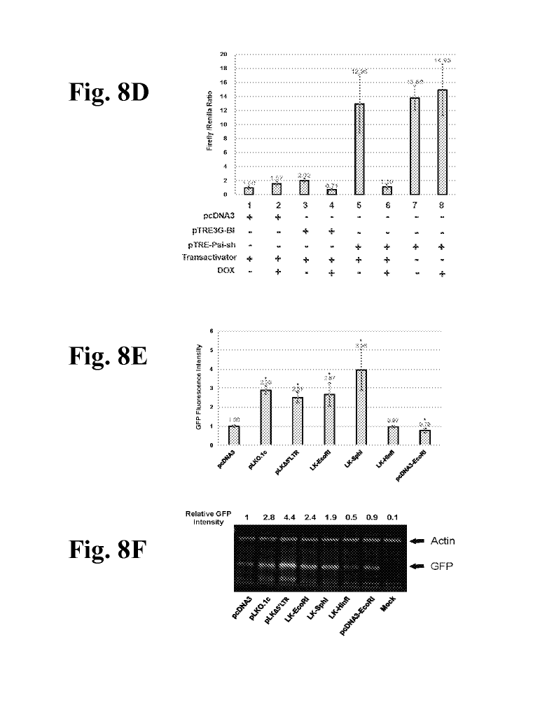

To assess whether the transfected DNA or their resulting transcripts increases expression of co-transfected genes, we cloned the Psi to shRNA sequences from pLKO.1c into the vector pTRE3G-BI-mCherry, which contains a bidirectional doxycycline (DOX)-inducible promoter (FIG. 8A). DOX addition induces a conformational change in the transactivator, resulting in promoter binding and upregulation of gene expression. The derivative vector (pTRE-Psi-sh) was co-transfected with an EGFP expression vector into 293 cells in the presence and absence of the transactivator (FIG. 8B). As expected in the presence of DOX, mCherry was induced in both the parental pTRE3G vector as well as the derivative pTRE-Psi-sh. Furthermore, the inclusion of the Psi-sh sequences increased mCherry expression ˜3.5 fold in the absence of the transactivator compared to pTRE3G-BI-mCherry, indicating that superinduction occurs in cis when the promoter is inactive.

To determine whether expression of the lentiviral sequences was required for superinduction in trans, we co-transfected either pTRE3G or pTRE-Psi-sh and the EGFP expression vector. GFP expression was slightly increased by the presence of pTRE3G, whereas 6-fold superinduction was observed with pTRE-Psi-sh in the absence of the transactivation and DOX (FIG. 8C). Promoter activation by DOX and the transactivator greatly suppressed EGFP expression in the presence of either the parental or pTRE-Psi-sh vector, suggesting that superinduction was eliminated when transcription was activated. This result was corroborated by co-transfection experiments using the bicistronic reporter (FIG. 8D) instead of EGFP. Cap-dependent translation of firefly luciferase only was favored when either DOX or the transactivator were absent and transcription was inactivated. Finally, deletion of the 5′ LTR promoter from pLKO.1c did not affect superinduction of co-transfected EGFP nor did linearization of the plasmid DNA with either Sphl or EcoRI as measured by FACS or Western blotting (FIGS. 8E and F). In contrast, cleavage of pLKO.1c into fragments of ˜1 kb or less with HinfI eliminated superinduction of EGFP. These experiments are consistent with superinduction of cap-dependent translation by circular or linear DNA. Indeed, our results are consistent with superinduction by a distinct DNA structure.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention should not be unduly limited to such specific embodiments. Indeed, various modifications of the described modes for carrying out the invention that are obvious to those skilled in molecular biology, diagnostics, medicine, or related fields are intended to be within the scope of the present invention and the following Claims.

1. A method for transfecting a eukaryotic host cell, said method comprising co-transfecting into said eukaryotic host cell a first vector comprising a gene of interest in operable combination with a promoter, and a second vector comprising DNA sequences corresponding to highly structured RNA, under conditions such that said gene of interest is expressed in an amount that is greater than the level of expression where said first vector is transfected alone.

2. The method of claim 1, wherein said highly structured RNA comprises shRNA.

3. The method of claim 2, wherein said second vector further comprises a retroviral packaging element.

4. The method of claim 3, wherein said packaging element is the packaging signal ψ.

5. The method of claim 3, wherein said second vector further comprises a Rev-responsive element (RRE).

6. The method of claim 3, wherein said second vector further comprises the retroviral 5′ UTR.

7. The method of claim 5, wherein said second vector further comprises a promoter upstream of the shRNA.

8. The method of claim 6, wherein said promoter is the U6 promoter.

9. The method of claim 7, wherein said second vector comprises a multiple cloning site and said U6 promoter is positioned in said multiple cloning site of the second vector.

10. The method of claim 1, wherein said second vector is a plasmid vector.

11. The method of claim 1, wherein said second vector is a viral vector.

12. The method of claim 10, wherein said viral vector is a retrovirus vector.

13. The method of claim 11, wherein said retrovirus vector is a lentiviral vector.

14. The method of claim 12, wherein said lentiviral vector is not capable of making viral particles.

15. The method of claim 11, wherein said retrovirus vector is a gammaretroviral vector.

16. The method of claim 14, wherein said gamaretroviral vector is not capable of making viral particles.

17. The method of claim 1, wherein said eukaryotic host cell is a primary cell.

18. The method of claim 1, wherein said eukaryotic host cell is a non-dividing cell.

19. The method of claim 1, wherein said eukaryotic host cell is part of a human tissue and said co-transfecting is done in vivo.

20. The method of claim 1, wherein said co-transfecting is done ex vivo and said transfected host cell is introduced into a human in vivo.

21. The method of claim 1, wherein said first vector is a plasmid.

22. The method of claim 20, wherein said plasmid comprises a DNA vaccine wherein said gene of interest encodes an antigen.

23. The method of claim 21, wherein said DNA vaccine is a preventative vaccine for viral infection.

24. The method of claim 22, wherein said preventative vaccine is for West Nile virus.

25. The method of claim 22, wherein said preventative vaccine is for influenza.

26. The method of claim 24, wherein said antigen is the influenza hemagglutinin antigen.

27. The method of claim 21, wherein said DNA vaccine is a therapeutic vaccine.

28. The method of claim 26, wherein said therapeutic vaccine is a therapeutic cancer vaccine.

29. The method of claim 1, wherein said co-transfections are performed by electroporation.

30. The method of claim 1, wherein said co-transfections are performed by needle free jet delivery.

31. The method of claim 1, wherein said co-transfections are performed by lipid-based carriers.

32. The method of claim 21, wherein said DNA vaccine comprises a eukaryotic region that directs expression of said gene of interest and a bacterial region that provides selection and propagation in bacteria.

33. The method of claim 31, wherein said bacterial region provides selection and propagation in E. coll.

34. The method of claim 31, wherein said eukaryotic region comprises said promoter upstream, and a polyadenylation signal (polyA) downstream, of said gene of interest.

35. The method of claim 33, wherein said promoter is the constitutive human Cytomegalovirus (CMV) promoter.

36. The method of claim 1, wherein said first vector is an adenoviral vector.

37. The method of claim 1, wherein said DNA sequences corresponding to highly structured RNA are not transcribed.

38. A method for transfecting a eukaryotic host cell, said method comprising co-transfecting into said eukaryotic host cell comprising a first vector comprising a gene of interest in operable combination with a promoter, and a second vector comprising in operable combination the retroviral packaging signal psi (ψ,) the Rev-responsive element (RRE), the U6 promoter upstream of a shRNA site, and an shRNA site comprising DNA sequences encoding shRNA, wherein said gene of interest is expressed in an amount that is greater than the level of expression where said first vector is transfected alone.

39. A host cell that has been co-transfected with a first vector comprising a gene of interest in operable combination with a promoter, and a second vector expressing highly structured RNA.

40. The host cell of claim 39, wherein said highly structured RNA comprises shRNA.

41. A kit comprising a) a vector comprising in operable combination the retroviral packaging signal v, the Rev-responsive element (RRE), the U6 promoter upstream of an shRNA site, and an shRNA site comprising DNA sequences encoding an shRNA, and b) instructions for co-transfecting said vector with another vector comprising a gene of interest under conditions such that said gene of interest is expressed at higher levels than those achieved without co-transfection.
